(19) United States
(12) Patent Application Publication
(10) Pub. No.: US 2007/0026478 A1
(43) Pub. Date: Feb. 1, 2007

(54) Hemiasterlin affinity probes and their uses

(75) Inventors: Lee M. Greenberger, Montclair, NJ (US); Malathi Hari, Canton, MI (US); Maria Nunes, Park Ridge, NJ (US); Semiramis Ayral-Kaloustian, Tarrytown, NY (US); Arie Zask, New York, NY (US); Joseph L. Wooters, Brighton, MA (US)

Correspondence Address: DARBY & DARBY P.C., P.O. BOX 5257, NEW YORK, NY, 10150-5257, US

(73) Assignee: WYETH, MADISON, NJ (US)

(21) Appl. No.: 10/536,262

(22) PCT Filed: Nov. 21, 2003
(86) PCT No.: PCT/US03/37393

§ 371 Date: Feb. 24, 2006

Related U.S. Application Data
(60) Provisional application No. 60/428,050, filed on Nov. 21, 2002.

(51) Int. Cl. C12Q 1/37; A61K 38/06; C07K 5/06

(52) U.S. Cl. 435/23; 514/18; 530/331

(57) ABSTRACT

Photoaffinity probes are provided that are based hemiasterlin and derivative compounds thereof. Use of these probes to identity ending sites for these and other drugs, particularly anti-tubulin drugs, are also provided as are methods for identifying new drugs (e.g., new anti-tubulin drugs) that bind to these binding sites.

### 1. CROSS-REFERENCE TO RELATED APPLICATIONS

Priority is claimed under 35 U.S.C. 119(e) to copending U.S. provisional patent application Ser. No. 60/428,050 filed on Nov. 21, 2002. The contents of this prior application are hereby incorporated by reference and in their entirety.

### 2. FIELD OF THE INVENTION

The present invention relates to methods and compositions for identifying anticancer drugs and, in particular, for identifying binding sites and/or targets for anticancer drugs. In particular, the invention provides photoaffinity probes that mimic the binding of hemiasterlin derivatives, including the hemiasterlin derivative HTI-286, to tubulin. The invention also relates to methods for using such probes—including methods for identifying drug binding sites on tubulin, as well as diagnostic and prognostic methods that use these probes to identify cells containing mutant tubulin such as tumor cells. The invention additionally relates to methods using target binding sites that are identified with such probes; e.g., to identify new binding compounds and potential therapeutic compounds, and/or to identify potentially drug resistant cells and tumors.

### 3. BACKGROUND OF THE INVENTION

α- and β-tubulin heterodimers polymerize to form microtubules which are vital for mitosis, motility, secretion, and proliferation (Rowinksy and Tolcher, in Cancer Principles and Practice (Devita et al., eds.) 6^{th} Ed. 2001, 431-452). Agents that bind tubulin and disrupt the function of microtubules are of great interest as some of these agents are routinely used to treat cancer. Three well-defined classes of drugs that bind tubulin have been previously identified: (1) colchicine, (2) vinca alkaloids and (3) taxanes (Hamel, Med. Res. Rev. 1996, 16:207-231). Colchicine and vinca alkaloids bind to distinct sites in tubulin, prevent the formation or extensions of microtubules, and therefore induce the depolymermization of microtubules (Hamel, Med. Res. Rev. 1996, 16:207-231; Downing, Annu. Rev. Cell Dev. Biol. 2000, 16:89-111). Photoaffinity-labeling and electron microscopy studies have revealed that taxanes bind to a distinct site within β-tubulin (Downing, Annu. Rev. Cell Dev. Biol. 2000, 16:89-111). At stoichiometric amounts in relation to tubulin, taxanes stabilize microtubules and therefore actually enhance polymerization (Jordan, Curr. Med. Chem. Anti-Canc. Agents 2002, 2:1-17). However, at low concentrations, which are sufficient to inhibit cell division, all antimicrotubule agents alter microtubule dynamics without causing marked depolymerization or polymerization effects (Jordan, Curr. Med. Chem. Anti-Canc. Agents 2002, 2:1-17).

Vinca alkaloids and taxanes such as paclitaxel and docetaxel have been widely used to treat solid tumors. However, resistance to paclitaxel and vinca alkaloids is easily demonstrated in tissue culture systems and occurs frequently either at the onset or during the course of multiple cycles of chemotherapy in patients (Rowinksy and Tolcher, in Cancer Principles and Practice (Devita et al., eds.) 6^{th} Ed. 2001, 431-452). In tissue culture systems, vinca alkaloids, paclitaxel, and docetaxel, are excellent substrates for the ABC drug efflux pump, P-glycoprotein. This protein can be overexpressed in tumor cells in response to chemotherapeutic drugs and is believed to mediate resistance to these agents.

Recently a fourth class of anti-tubulin compounds, known as hemiasterlins, has been described. See, for example, Talpir et al., Tetrahedron Lett. 1994, 35:4453-4456; Gambel et al., Bioorg. Med. Chem. 1999, 7:1611-1615; Coleman et al., Tetrahedron 1995, 51:10653-10662; and Anderson et al., Cancer Chemother. Pharmacol. 1997, 39:223-226. Hemiasterlins, which are tripeptide compounds isolated from marine sponges, induce microtubule depolymerization, cell cycle arrest and ultimately cell death (Anderson et al., Cancer Chemother. Pharmacol. 1997, 39:223-226; Talpir et al., Tetrahedron Lett. 1994, 35:4453-4456; Hamel and Covel, Curr. Med. Chem. Anti-Canc. Agents 2002, 2:19-53). The use of hemiasterlin compounds in cancer therapy has also been described. See, for example, International Patent Publication Nos. WO 99/32509 and WO 96/33211. See also, U.S. Pat. No. 6,153,590. Methods for obtaining hemiasterlin compounds have additionally been described, both by isolating the compounds from marine sponges (U.S. Pat. Nos. 5,661,175 and 6,153,590) and by chemical synthesis (Anderson & Coleman, Tetrahedron Lett. 1997, 38:317-320).

Synthetic hemiasterlin analogs and derivative compounds have also been described (see, for example, International Patent Publication No. WO 99/32509) and these compounds also have cytotoxic and anti-mitotic activity. In particular, provisional U.S. patent application Ser. Nos. 60/411,883 and 60/493,841 filed on Sep. 20, 2002 and Aug. 8, 2003, respectively, describe various hemiasterlin derivative compounds, including one compound known as HTI-286.

HTI-286, which has the chemical structure set forth in Formula I, below, has a weak interaction with P-glycoprotein and has also been shown to overcome resistance of other anti-tubulin drugs, such as taxanes, both in vitro and in xenograft tumor models (Loganzo et al., Cancer Res. 2003, 63:1838-1845). Clinical trials of HTI-286 in cancer patients are in progress (Ratain et al., Proc. Am. Soc. Clin. Onc. 2003, abstract 516).

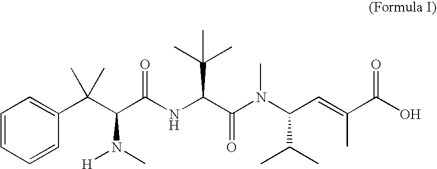

While hemiasterlins and it analogs/derivatives represent a promising class of new anti-tubulin drugs, it is expected that resistance to this drug and other anti-microtubule agents will continue to be a problem. This is because resistance to antimicrotubule agents is mutifactorial and is due to transporter pumps, alterations in tubulin, as well as changes in mechanisms that mediate cell death after microtubules have been disrupted (Dumontet et al., J. Clin. Oncol. 1999, 17:1061-1070). Therefore, the identification of new antimicrotubules agents that overcome resistance mechanisms may be useful clinically.

In order to identify such agents an understanding of the intracellular drug interactions (for example, with tubulin) is necessary. Unlike other anti-tubulin compounds, however, the tubulin binding site for hemiasterlins and their analogs (e.g., HTI-286) as well as any peptide-based inhibitors remains unknown and the exact mechanism(s) by which these compounds disrupt tubulin polymerization is poorly understood, at best (Hamel and Covel, Curr. Med. Chem. Anti-Canc. Agents 2002, 2:19-53). Knowledge of the binding site and mechanism(s) of action would greatly facilitate the design and identification of new, more effective, anti-tubulin compounds, as well as mutations (such as in α- and/or β-tubulin) that are associated with resistance to hemiasterlins. Hence, there continues to exist a need for anti-tubulin compounds, including new hemiasterlin derivatives or other peptide-like inhibitors, that are effective for inhibiting cancer and tumor cell growth. There also exists a need for methods (including diagnostic and prognostic methods) for identifying tumor cells and, in particular, for identification cell lines and/or mutations that are resistant to such hemiasterlin compounds.

The citation and/or discussion of a reference in this section and throughout the specification is provided merely to clarify the description of the present invention and is not an admission that any such reference is “prior art” to the invention described herein.

### 4. SUMMARY OF THE INVENTION

The present invention at least partly overcomes the above-described problems by providing compounds that are useful, e.g., for detecting hemiasterlin binding sites. In addition, the invention also provides binding data from such probes that can be used, inter alia, to design novel hemiasterlin compounds. Such hemiasterlin compounds are themselves useful, e.g., as new anti-cancer drugs. Binding data obtained using compounds of the invention can also be used to identify cells (particularly cancer cells) that are resistant to or are likely to be resistant to hemiasterlin drugs. Such methods therefore are also provided in the present invention.

The invention therefore provides, more specifically, compounds represented by the chemical formual:

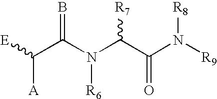

or a pharmaceutically acceptable salt thereof, in which at least on of the substituents A, B, E, R₆, R₇, R₈ and/or R₉ comprises a photoreactive group such as a benzophenone or an azide moiety and in which the substituents A, B, E, R₆, R₇ R₈ and R₉, when not a photoreactive moiety, is as described in the Sections below.

Preferred compounds of the invention have the chemical formula:

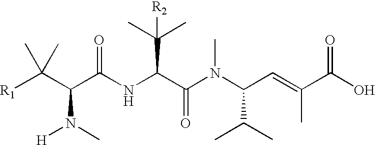

or a pharmaceutically acceptable salt thereof in which R₁ is either a photoreactive moiety or an aryl moiety, R₂ is either a photoreactive moiety, and alkyl moiety of H, and wherein at least one of R₁ and R₂ comprises a photoreactive moiety.

Particularly preferred compounds of the invention are:
4-benzoyl-N,β,β-trimethyl-L-phenylalanyl-N1-[(1S,2E)-3-carboxy-1-isopropylbut-2-enyl]-N1,3-dimethyl-L-valinamide;
N,β,β-trimethyl-L-phenylalanyl-4-benzoyl-N-[(1S,2E)-3-carboxy-1-isopropyl-2-butenyl]-N,β,β-trimethyl-L-phenylalaninamide;
   and pharmaceutically acceptable salts thereof. Preferred compounds of the invention also include ones having the chemical formula: 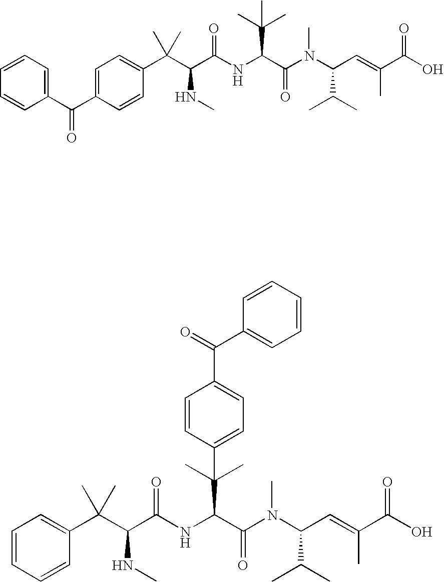
   and pharmaceutically acceptable salts thereof.

The invention also provides methods that use such compounds to identify tubulin binding sites, e.g., for hemiasterlin drugs. Such methods generally provide contacting a compound of the invention (e.g., one of the compounds described, supra) to a sample containing tubulin such that the compound can irreversible bind tubulin in the sample. The tubulin is then separated into a plurality of different tubulin fragments, and at least one tubulin fragment is identified that has the compound bound thereto. Those fragments that have a compound of the invention bound to them are thereby identified as fragments that comprise a tubulin binding site.

The invention additionally provides methods for identifying hemiasterlin competitors—i.e., compounds that compete with a hemiasterlin compound for binding to tubulin. Such compounds can themselves be useful, e.g., as novel anti-tubulin and anti-cancer drugs. The methods generally involve contacting both a test compound and a probe, which is one of the compounds of the invention (e.g., any of the compounds described supra), is contacted to a sample that contains tubulin, and binding of the probe to tubulin is detected. This binding is compared to binding of the probe to tubulin that is observed in the absence of the test compound. Where binding in the presence of the test compound is lower than in the absence of the test compound, then the test compound is identified as a hemiasterlin inhibitor.

In various aspects of these different embodiments, the compound of the tubulin sample is preferably irradiated (e.g., with UV-light) after being contacted with the probe compound, e.g., to promote irreversible, covalent binding of the probe compound to tubulin. The probe compound is also preferably labeled so that its binding to tubulin (or to a tubulin fragment) can be detected by detecting the label. In other aspects of these methods, tubulin in the sample can be separated, e.g., by chemical digest (for example, with formic acid or CNBr) or by enzymatic digestion (for example, with Lys C, Trypsin or subtilisin).

These and other particular embodiments of the invention are described in detail in the following Sections.

### 5. BRIEF DESCRIPTION OF THE DRAWINGS

FIG. 1A illustrates results of purified bovine brain tubulin (2.5 μM) and purified Hela cell tubulin (10 μM) incubated for 30 minutes at room temperature with 2.5 μM (3.6 μCi) of [³H]-probe 1. After incubation, the samples were irradiated for 2 hours at 4° C. with 360 nm UV light. Proteins were then resolved by SDS-PAGE and analyzed by Coomassie blue staining (left) or fluorography (right) of the gel as described below.

FIG. 1B illustrates the labeling of KB-3-1 cell lysates (50 μg) (lanes 1 and 2) and purified bovine brain (BB) tubulin (5 μg) (lanes 3 and 4) incubated for 30 minutes at room temperature with 2.5 μM [³H]-probe 1. Samples in lanes 1 and 3 were pre-incubated with 250 μM non-radiolabeled probe 1 prior to incubation with radiolabeled materials. Samples were analyzed as described in FIG. 1A. Lanes marked “M” are molecular weight markers in kDa.

FIG. 2 illustrates the inhibition of α-tubulin labeling by Probe 1 in the presence of HTI-286. The gel image resulted from the following procedure: Bovine brain tubulin (2.5 μM) was incubated without or with 1 mM HTI-286 for 15 min at 4° C., followed by addition of increasing concentrations of [³H]-probe 1 (0.025-2.5 μM) (0.036-3.6 μCi). After incubation for 30 minutes at 37° C., samples were irradiated with 360 nm UV light for 30 minutes and separated by SDS-PAGE. Gels were exposed to film by fluorography.

FIGS. 3A-3C provide data for the digestion of Probe 1-photolabeled tubulin by formic acid. Photolabeled tubulin was resolved by SDS-PAGE under conditions that allowed α- and β-tubulin to comigrate. The tubulin band was excised from the gel and digested with 75% formic acid at 37° C. After 44 or 72 hours, formic acid digestion products were separated on a 10-20% Tris-tricine gel. FIG. 3A shows a diagram of the predicted formic acid cleavage sites and cleavage products for α-tubulin. FIGS. 3B and 3C are visualizations of the gel fragments stained by Coomassie Blue and of the radioactive fragments detected by fluorography, respectively.

FIGS. 4A-4D provide data for the digestion of Probe 1-photolabeled tubulin with trypsin. FIG. 4A is a diagram demonstrating known cleavage sites within α-tubulin after native tubulin digestion by trypsin. FIGS. 4B and 4C are images of trypsin cleavage fragments observed after Coomassie Blue staining or fluorography methods, respectively. For these images, tubulin (15 μg) was incubated with 0.25 μM (0.36 μCi) of [³H]-Probe 1 for 30 minutes at room temperature and irradiated at 4° C. with 360 nm UV light. After 2 hours, the samples were digested with 0.8 μg trypsin at 30° C. for 0 to 30 minutes. After stopping the reaction with 0.01 mM leupeptin, samples were resolved on 10-20% Tris-tricine gels. Alternatively, as demonstrated in FIG. 4D, 1 mm gel slices were removed from the gel described in FIG. 4C and quantitated to provide a radioactivity profile of the length of the gel.

FIGS. 5A-5D provide data for the digestion of Probe 1 photolabeled tubulin by CNBr. FIGS. 5A and 5B show images of the CNBr-digested tubulin fragments visualized by either fluorography or silver staining, respectively. These gel images were produced as follows: After native tubulin was photolabeled as described in Section 6.1.5, α- and β-tubulin were visualized in gels by Coomassie blue staining. Protein within each species was extracted, reduced, alkylated, and digested with 150 mg/mL CNBr in 70% formic acid at 37° C. After 48 hours, samples were resolved by SDS-PAGE. FIG. 5C diagrams the predicted position of the 7 kDa labeled peptide fragment from α-tubulin obtained after CNBr digestion. FIG. 5D shows all predicted CNBr fragments of rat α-tubulin. The boxes indicate sequences that are confirmed using mass spectrometry.

FIGS. 6A-6C provide data for the digestion of tubulin by subtilisin before or after photoaffinity labeling with Probe 1. FIG. 6A diagrams the predicted subtilisin cleavage site and cleavage products for α-tubulin. FIG. 6B provides Coomassie staining (top) and fluorographic (bottom) images of fragments photolabeled with Probe 1 and exposed to subtilisin as follows: Tubulin (5 μg) was incubed without (Lanes 1 and 4) or with 0.2 μg subtilisin for 60 minutes at 30° C. and then photolabeled with 0.25 μM [³H]-Probe 1 (Lane 2) according to conditions specified in Section 6.1.11. Alternatively, photolabeling was done before subtilisin digestions (Lane 3). In both cases samples were then separated by SDS-PAGE. FIG. 6C shows Commassie staining and fluorographic images of tubulin fragments prepared as follows: tubulin (5 μg) was pre-incubated with 0, 100, or 250 μM non-radiolabeled probe 1 for 15 minutes, labeled with [³H]-Probe 1 as described above, and then digested with 0.2 μg subtilisin for 60 minutes at 30° C. as described in Section 6.1.11. Again, samples were separated by SDS-PAGE.

FIGS. 7A-7B present data for the photolabeling of bovine brain tubulin (Lane 1) and HeLa cell tubulin (Lane 2) using Probe 2 as described in Sections 6.1.5 and 6.6.1. FIG. 7A provides an image of a 7.5% Tris-HCl gel of bovine brain and HeLa cell tubulin stained with Commassie blue. FIG. 7B shows an audioradiograph of the gel in FIG. 7A.

FIGS. 8A-8E present data for trypsin, subtilisin, formic acid, and Lys C digestions of purified bovine brain tubulin before or after photolabeling with Probe 2. Methods for these procedures are described in Sections 6.1.7, 6.1.12, and 6.1.13. FIGS. 8A and 8B provide Commassie blue stained gel and fluorographic images, respectively, of tubulin fragments from trypsin, subtilisin, and formic acid digestions. FIG. 8B shows a fluorographic image of fragments that have been radiolabeled with Probe 2. Similarly, FIGS. 8C and 8D provide Commassie blue stained gel and fluorographic images, respectively, of tubulin fragments from Lys C digestions. FIG. 8D shows a fluorographic image of fragments that have been radiolabeled with Probe 2. FIG. 8E diagrams the predicted Lys C fragments (described by their molecular weight, length, and sequence) of rat α-tubulin. The 12.8 kDa fragment is in bold.

FIGS. 9A-9B provide data for the digestion of Probe 2-labeled bovine brain tubulin using CNBr. Probe 2 labeled tubulin was separated into α and β-tubulin subunits by SDS-PAGE and separately digested with CNBr. FIG. 9A displays a silver stained 4-20% Tris-Tricine gel of tubulin fragments after CNBr digestion. FIG. 9A shows an audioradiograph containing radiolabeled fragments prepared according to Section 6.1.8.

FIG. 10 displays Commassie blue stained 7.5% Tris-HCl SDS-PAGE gels of purified bovine brain tubulin incubated with 50 μM drug and digested with subtilisin for various times. Reactions were quenched and fragments were separated according to Section 6.1.14.

FIG. 11 is a structural model depicting the αβ-tubulin dimer. The bracketed areas show the approximate regions for the α- and β-tubulin subunits. The general area of the Probe 1 and Probe 2 binding regions are identified with arrows. The bound GDP, GTP and paclitaxel are shown as ball and stick models. (PDB accession number 1JFF).

FIG. 12 shows a protein sequence alignment for amino acid residues 1-451 of human (Homo sapien, designated htub1 and htub2), primate (Macaca fascicularis and Macaca mulatta, designated ptub1 and ptub2 respectively), mouse (Mus musculus, designated motub1), hamster (Cricetulus griseus, designated hamtub1), rat (Rattus norvegicus, designated rattub1), chicken (Gallus gallus, designated chicktub1), and frog (Xenopus laevis, designated frogtub1) α-tubulin (Stachi et al., Biochem. Biophys. Res. Commun. 2000, 270:1111-1118). The EMBL Accession Numbers for htub1, htub2, ptub1, ptub2, motub1, hamtub1, rattub1, chicktub1, and frogtub1 are AJ245922, K00558, X04757, AF141923, AJ245923, M12329, V01227, M16030, and X07046, respectively.

### 6. DETAILED DESCRIPTION

6.1. Affinity Probes

The present invention provides compounds that are generally referred to here as “affinity probes” and which are useful to detect and identify binding sites for drugs—particularly anti-cancer drugs. In particular, affinity probes of this invention are useful to detect and identify binding sites for a class of anti-cancer drugs referred to here as “anti-tubulin” drugs or, alternatively, as “tubulin inhibitors” or “tubulin binding compounds.” Without being limited to any particular theory or mechanism of action, tubulin inhibitors are believed to exert their therapeutic effects by specifically binding tubulin (which can be either α-tubulin or β-tubulin) and disrupting the polymerization of these subunits into microtubules in cells. Hence, affinity probes of this invention are useful for identifying binding sites of tubulin inhibitor drugs on tubulin.

Preferred affinity probes of the invention are analogs of a particular class of anti-tubulin drugs generally referred to as hemiasterlin derivatives or “hemiasterlins.” Hemiasterlins are natural products derived from marine sponges that induce microtubule depolymerization, cell cycle arrest and ultimately cell death (Anderson et al., Cancer Chemother. Pharmacol. 1997, 39:223-226; Telpin et al., Tetrahedron Letters 1994, 35:4453-4456). The use of hemiasterlin compounds in cancer therapy has also been described. See, for example, International Patent Publication Nos. WO 99/32509 and WO 96/33211. See also, U.S. Pat. No. 6,153,590. Methods for obtaining hemiasterlin compounds have additionally been described, both by isolating the compound from marine sponges (U.S. Pat. Nos. 5,661,175 and 6,153,590) and by chemical synthesis (Anderson & Coleman, Tetrahedron Letters 1997, 38:317-320).

Synthetic hemiasterlin analogs and derivative compounds have also been described (see, for example, International Patent Publication No. WO 99/32509) and these compounds also have cytotoxic and anti-mitotic activity. Accordingly, such hemiasterlin derivative and anglogue compounds are also considered hemiasterlins, at least for the purposes of describing and claiming this invention. It is to be understood that, for the purposes of describing this invention, the terms “hemiasterlin derivative” and “hemiasterlin analog” are used interchangeably. Both these terms (in all their variants) therefore generally refer to compounds that are derived from and/or are chemical analogs of a natural hemiasterlin compound.

Particularly preferred classes of hemiasterlin derivatives have been described in provisional U.S. patent application Ser. Nos. 60/411,883 and 60/493,841 filed on Sep. 20, 2002 and Aug. 8, 2003, respectively. These include a particular hemiasterlin derivative known as HTI-286, which has the chemical structure set forth in Formula I, below (see also Table I in the examples, infra).

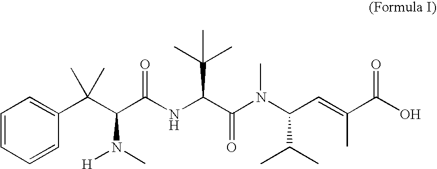

HTI-286 has a weak interaction with P-glycoprotein and has been shown to overcome resistance of other anti-tubulin drugs (e.g., taxane) both in vitro and in xenograft tumor models (Loganzo et al., Cancer Res. 2003, 63:1838-1845). The compound is therefore a particularly useful as an anti-cancer drug, and clinical trial of HTI-286 in cancer patients are in progress (Ratain et al., Proc. Am. Assoc. Cancer Res. 2003, abstract 516). Hence, affinity probes of the present invention that are analogs of HTI-286 are particularly preferred.

Accordingly, the present invention provides compounds that are hemiasterlin derivatives as set forth in U.S. provisional patent application Ser. Nos. 60/411,883 and 60/493,841 filed on Sep. 20, 2002 and Aug. 8, 2003 respectively in which one or more chemical moiety or substitutent is either replaced by or additionally comprises a photoreactive group. Examples of preferred photoreactive groups which may be present in a compound of the invention include an azide moiety and a benzophenone moiety, with benzophenone moieties being preferred. Other photoreactive groups include, but are not limited to, diazo groups, diazirines, enones, sulfur radicals, halogenated substrates, nitrobenzenes, dizonium salts, sulfonium salts, as well as those groups cited by Fleming (Tetrahedron 1995, 51:12479-12520).

For example, the present invention provides compounds represented by Formula II, below: 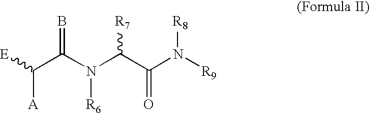
wherein at least one of the substituents A, B, E, R₆, R₇, R₈ and/or R₉ comprises a photoreactive group such as an azide or a benzophenone moiety. In preferred embodiments, at least one of the substitutents A, B, E, R₆, R₇, R₈ and/or R₉ is a benzophenone moiety.

In Formula II, above, A can comprise a photoreactive group and/or is selected from the group consisting of an alkyl moiety of 1 to 10 carbon atoms, an alkenyl moiety of 2 to 10 carbon atoms, an aryl and a cyclic hydrocarbon moiety of 3 to 10 carbon atoms, wherein carbon atoms may optionally be replace with 0 to 4 nitrogen atoms, 0 to 4 oxygen atoms, and 0 to 4 sulfur atoms, and the carbon atoms are optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, —Br, —Cl, —F, CN, —CO₂H, —CHO, —COR₁₀, —CONH₂, —CONHR₁₀, CON(R₁₀)₂, —COSH, —COSR₁₀, NO₂, —SO₃H, SOR₁₀, —SO₂R₁₀ wherein R₁₀ is an alkyl moiety of 1 to 10 carbon atoms, an alkenyl moiety of 2 to 10 carbon atoms, and aryl and a cyclic hydrocarbon moiety of 3 to 10 carbon atoms, aryl-R— or heteroaryl-R; or A can be OR, S(O)R, S(O)₂R, SO₂NR₂, NR₁R₂ or N₃.

R can comprise a photoreactive group and/or is selected from the group consisting of H, an alkyl moiety of 1 to 18 carbon atoms, an alkenyl moiety of 2 to 18 carbon atoms, an aryl and a cyclic hydrocarbon moiety of 3 to 18 carbon atoms, wherein carbon atoms may optionally be replaced with 0 to 4 nitrogen atoms, 0 to 4 oxygen atoms, and 0 to 4 sulfur atoms, and the carbon atoms are optionally substituted with ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, —Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀ or —SO₂R₁₀; wherein R₁₀ is an alkyl moiety of 1 to 10 carbon atoms, an alkenyl moiety of 2 to 10 carbon atoms, and aryl and a cyclic hydrocarbon moiety of 3 to 10 carbon atoms, aryl-R— or heteroaryl-R.

B is O or H₂.

E can comprise a photoreactive group and/or is the moiety:

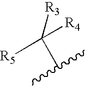

Alternatively, E can comprise a photoreactive group and/or comprises an aryl moiety, a 5 to 14-membered monocyclic, bicyclic or tricyclic saturated or unsaturated hydrocarbon ring moiety wherein carbon atoms may optionally be replaced with 0 to 4 nitrogen atoms, 0 to 4 oxygen atoms, and 0 to 4 sulfur atoms wherein the carbon atoms may optionally be substituted with: R, ═O, ═S, —OH, —OR₁₀, —O₂CR₁₀, —SH, —SR₁₀, —SOCR₁₀, —NH₂, —NR₁₀H, —N(R₁₀)₂, —NHCOR₁₀, —I, —Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀ or —SO₂R₁₀;

R₁ can comprise a photoreactive group and/or a moiety is selected from the group consisting of H, an alkyl moiety of 1 to 10 carbon atoms, alkenyl moiety of 2 to 10 carbon atoms, aryl and a cyclic hydrocarbon moiety of 3 to 10 carbon atoms, wherein carbon atoms may optionally be replaced with 0 to 4 nitrogen atoms, 0 to 4 oxygen atoms, and 0 to 4 sulfur atoms, and the carbon atoms may optionally be substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀;

R₂ can comprise a photoreactive group and/or a moiety selected from the group consisting of H, an alkyl moiety of 1 to 10 carbon atoms, alkenyl moiety of 2 to 10 carbon atoms, aryl and a cyclic hydrocarbon moiety of 3 to 10 carbon atoms, wherein carbon atoms may optionally be replaced with 0 to 4 nitrogen atoms, 0 to 4 oxygen atoms, and 0 to 4 sulfur atoms, and the carbon atoms may optionally be substituted with: ═O, ═S, OH, —OR₁₀, —O₂CR₁₀, —SH, —SR₁₀, —SOCR₁₀, —NH₂, —NR₁₀—H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀;

R₁ and R₂ together may optionally form a ring of 3 to 7 carbon atoms wherein carbon atoms may optionally be replaced with 0 to 2 nitrogen atoms, 0 to 2 oxygen atoms and 0 to 2 sulfur atoms;

R₃ can comprise a photoreactive group and/or a moiety selected from the group consisting of H, an alkyl moiety of 1 to 10 carbon atoms, alkenyl moiety of 2 to 10 carbon atoms, aryl and a cyclic hydrocarbon moiety of 3 to 10 carbon atoms, wherein carbon atoms may optionally be replaced with 0 to 4 nitrogen atoms, 0 to 4 oxygen atoms, and 0 to 4 sulfur atoms, and the carbon atoms may optionally be substituted with: ═O, —S, OH, —OR₁₀, —O₂CR₁₀, —SH, —SOCR₁₀, —NH₂, —NR₁₀H, —N(R₁₀)₂, —NHCOR₁₀, NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀;

R₄ can comprise a photoreactive group and/or a moiety selected from the group consisting of H, an alkyl moiety of 1 to 10 carbon atoms, alkenyl moiety of 2 to 10 carbon atoms, aryl and a cyclic hydrocarbon moiety of 3 to 10 carbon atoms, wherein carbon atoms may optionally be replaced with 0 to 4 nitrogen atoms, 0 to 4 oxygen atoms, and 0 to 4 sulfur atoms, and the carbon atoms may optionally be substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀;

R₃ and R₄ together may optionally form a ring of 3 to 7 carbon atoms wherein carbon atoms may optionally be replaced with 0 to 2 nitrogen atoms, 0 to 2 oxygen atoms and 0 to 2 sulfur atoms;

R₅ can comprise a photoreactive group and/or a moiety selected from the group consisting of H, OH, NHR, SH, aryl, heteroaryl, an alkyl moiety of 1 to 10 carbon atoms, alkenyl moiety of 2 to 10 carbon atoms and a cyclic hydrocarbon moiety of 3 to 10 carbon atoms, wherein carbon atoms may optionally be replaced with 0 to 4 nitrogen atoms, 0 to 4 oxygen atoms, and 0 to 4 sulfur atoms, and the carbon atoms may optionally be substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀;

R₅ and A may optionally form a ring of 5 to 7 carbon atoms wherein carbon atoms may optionally be replaced with 0 to 2 nitrogen atoms, 0 to 2 oxygen atoms, and 0 to 2 sulfur atoms;

R₆ can comprise a photoreactive group and/or a moiety selected from the group consisting of H, an alkyl moiety of 1 to 10 carbon atoms, alkenyl moiety of 2 to 10 carbon atoms, aryl and a cyclic hydrocarbon moiety of 3 to 10 carbon atoms, wherein carbon atoms may optionally be replaced with 0 to 4 nitrogen atoms, 0 to 4 oxygen atoms, and 0 to 4 sulfur atoms, and the carbon atoms may optionally be substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀;

R₇ can comprise a photoreactive group and/or a moiety selected from the group consisting of H, an alkyl moiety of 1 to 10 carbon atoms, alkenyl moiety of 2 to 10 carbon atoms, aryl and a cyclic hydrocarbon moiety of 3 to 10 carbon atoms, wherein carbon atoms may optionally be replaced with 0 to 4 nitrogen atoms, 0 to 4 oxygen atoms, and 0 to 4 sulfur atoms, and the carbon atoms may optionally be substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀;

R₈ can comprise a photoreactive group and/or a moiety selected from the group consisting of H, an alkyl moiety of 1 to 10 carbon atoms, alkenyl moiety of 2 to 10 carbon atoms, aryl and a cyclic hydrocarbon moiety of 3 to 10 carbon atoms, wherein carbon atoms may optionally be replaced with 0 to 4 nitrogen atoms, 0 to 4 oxygen atoms, and 0 to 4 sulfur atoms, and the carbon atoms may optionally be substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀;

R₉ can comprise a photoreactive group and/or a moiety selected from the group consisting of:

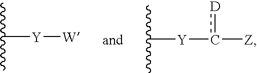

wherein W′ can comprise a photoreactive group and/or is selected from the group consisting of SO₂R₁₆, SO₃R₁₄, SO₂NR₁₄R₁₅, P(O)(OR₁₄)(OR₁₅), CN, OH, tetrazole, a moiety

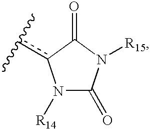

and SO₂NRR where the R groups may form a 4 to 8 membered ring wherein the carbon atoms may optionally be replaced with 0 to 2 nitrogen atoms, 0 to 2 oxygen atoms and 0 to 2 sulfur atoms.

R₁₄ and R₁₅ may comprise a photoreactive moiety and/or are independently selected from the group consisting of H and an alkyl moiety of 1 to 6 carbon atoms. R₁₆ is an alkyl moiety of 1 to 6 carbon atoms in length. D is O or OH and/or may comprise a photoreactive group.

Z and Y may optionally form a ring of 5 to 7 carbon atoms wherein the carbon atoms may optionally be replaced by 0 to 2 nitrogen atoms, 0 to 2 oxygen atoms and/or 0 to x sulfur atoms. Y may comprise a photoreactive moiety and/or may comprise an alkyl moiety of 1 to 10 carbon atoms optionally substituted with R, ArylR—, or X or an alkenyl moiety of 2 to 10 carbon atoms optionally substituted with R, ArylR— or X. Z may comprise a photoreactive moiety and/or may comprise a moiety selected from the group consisting of: H, an alkyl moiety of 1 to 6 carbon atoms, —NRN(R)₂, R, aryl, heteroaryl, aralkyl, —OR, —SH, —SR, —NH₂, —NHR, —NROR, —N(R)₂, NH—NH₂, and NRR; where the R group may form a 4 to 8 membered ring wherein carbon atoms may optionally be replaced with 0 to 2 nitrogen atoms, 0 to 2 oxygen atoms and/or 0 to 2 sulfur atoms, —NHCH(R₁₁)COOH; and —NRCH(R₁₁)COOH in which R₁₁ may comprise a photoreactive group and/or is a moiety having the formula R or —(CH2)ₙNR₁₂R₁₃ wherein n=1-4 and both R₁₂ and R₁₃ are independently selected from the group consisting of H, R and —C(NH)(NH₂).

Alternatively, Z may comprise a photoreactive moiety and/or may comprise a moiety having selected from:

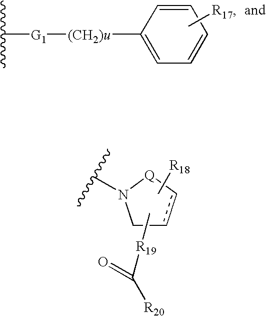

wherein the doted line indicates an optional chemical bond, Q is (CH₂)ₘ, G₁ is selected from the group consisting of O, N and S, m is an integer of between 1 and 3, and u is an integer of between 0 and 5. R₁₇ is a phenyl or O—(CH₂)ₙphenyl, R₁₈ is H or OH, and R₁₉ is selected from a chemical bond, an alkyl moiety of 1 to 10 carbon atoms optionally substituted with an alkyl moiety of 1 to 10 carbon atoms and alkoxy of 1 to 10 carbon atoms.

R₂₀ is selected from OR₁₄, NH—R₂₁, a moiety of the formula:

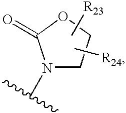

and a moiety of the formula:

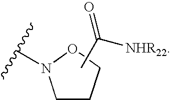

R₂₁ R₂₂ are each preferably an alkyl moiety of 1 to 10 carbon atoms optionally substituted with aryl and/or heteroaryl moieties.

X is defined as a moiety selected from the group consisting of: —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR and —SO₂R.

In preferred embodiments the invention provides a compound according to Formula II, as defined above, wherein at least one of the substituents R₃, R₄, R₅, and R₇ comprises a photoreactive group such as an azide moiety or a benzophenone moiety, with a benzophonenone moiety being particularly preferred. In particularly preferred embodiments a compound according to Formula II comprises a photoreactive group (preferably a benzophonenone moiety) at the substituent R₅ or at the substituent R₇.

The present invention also provides compounds represented by Formula III, below: 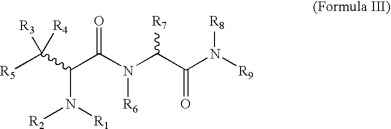
wherein at least one of the substituents R₁, R₂, R₃, R₄, R₅, R₆, R₇, R₈ and/or R₉ comprises a photoreactive group such as an azide or a benzophenone moiety. In preferred embodiments, at least one of the substitutents R₁, R₂, R₃, R₄, R₅, R₆, R₇, R₅ and/or R₉ is a benzophenone moiety.

In a compound of Formula III, above, R₁ can comprise a photoreactive group and or may comprise a moiety selected from the group consisting of: H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R—.

R₂ can comprise a photoreactive group and or may comprise a moiety selected from the group consisting of: H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R—.

Alternatively, R₁ and R₂ taken together with the nitrogen atom to which they are attached can be three to seven membered ring.

R₃ can comprise a photoreactive group and or may comprise a moiety selected from the group consisting of: H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R—.

R₄ can comprise a photoreactive group and or may comprise a moiety selected from the group consisting of: H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R—.

Alternatively, R₃ and R₄ taken together with the carbon to which they are attached can form a three to seven membered ring.

R₅ can comprise a photoreactive group and or may comprise a moiety selected from the group consisting of: H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; aryl-R— and aryl. In a preferred embodiment R₅ is an idolyl moiety of the formula: 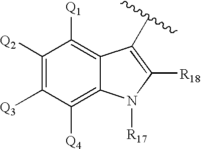
in which R₁₇ is H, a photoreactive group or an optionally substituted acyl group. R₁₈, Q₁, Q₂, Q₃ and Q₄ are independently selected from H, a photoreactive group, a halogen, alkyl, acyl, —OH, —O-alkyl, —O-acyl, —NH₂, —NH-alkyl, —N(alkyl)₂, —NH-acyl, —NO₂, —SH, —S-alkyl and —S-acyl in which the alkyl and acyl groups of the substituents are optionally substituted.

R₆ can comprise a photoreactive group and or may comprise a moiety selected from the group consisting of: H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, CO₂R₁₀, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R—.

R₇ can comprise a photoreactive group and or may comprise a moiety selected from the group consisting of: H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R—.

R₈ can comprise a photoreactive group and or may comprise a moiety selected from the group consisting of: H; a saturated or unsaturated moiety having a linear, branched, or cyclic skeleton containing one to ten carbon atoms, zero to four nitrogen atoms, zero to four oxygen atoms, and zero to four sulfur atoms, said carbon atoms being optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CH—O, —COR₁₀, —CONH₂, —CONHR₁₀, —CON(R₁₀)₂, —COSH, —COSR₁₀, —NO₂, —SO₃H, —SOR₁₀, or —SO₂R₁₀, wherein R₁₀ is a linear, branched or cyclic, one to ten carbon saturated or unsaturated alkyl group; and aryl-R—.

R₉ can comprise a photoreactive group and/or the moiety:

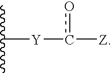

In the above formula, R is preferably a saturated or unsaturated moiety having a linear, branched, or cyclic skeleton containing one to ten carbon atoms, zero to four nitrogen atoms, zero to four oxygen atoms and zero to four sulfur atoms. The carbon atoms in R can be optionally substituted with: ═O, ═S, OH, —OR₁₀, —O₂CR₁₀, —SH, —SR₁₀, —NH₂, —NR₁₀H, —N(R₁₀)₂, —NHCOR₁₀, —NR₁₀COR₁₀, —I, Br, —Cl, —F, —CN, —CO₂H, —CO₂R₁₀, —CHO, —COR₁₀, —CONH₂, —CONHR₁₀, —CON(R₁₀)₂, —COSH, —COSR₁₀, —NO₂, —SO₃H, —SOR₁₀, —SO₂R₁₀ wherein R₁₀ is a linear, branched or cyclic, one to ten carbon saturated or unsaturated alkyl group.

X may be a photoreactive group and/or comprises a moiety selected from the group consisting of: —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R.

Y may be a photoreactive group and/or comprises a moiety selected from the group consisting of: a linear, saturated or unsaturated on to six carbon alkyl group that is optionally substituted with R, ArylR—, or X.

Z may be a photoreactive group and/or comprises a moiety selected from the group consisting of —OH, —OR, —SH, —SR, —NH₂, —NHR, —N(R)₂, —NHCH(R₁₁)COOH. R₁₁ may comprise a photoreactive group and/or is a moiety having the formula R or —(CH₂)ₙNR₁₂R₁₃, where n is an integer of between 1 and 4, and where R₁₂ and R₁₃ are independently selected from the group consisting of H, R, —C(NH)(NH₂) and pharmaceutically acceptable salts thereof.

In preferred embodiments the invention provides a compound according to Formula II, as defined above, wherein at least one of the substituents R₃, R₄, R₅, and R₇ comprises a photoreactive group such as an azide moiety or a benzophenone moiety, with the benzophonenone moiety being particularly preferred. In particularly preferred embodiments a compound according to Formula II comprises a photoreactive group (preferably a benzophonenone moiety) at the substituent R₅ or at the substituent R₇.

In particularly preferred embodiments the invention provides compounds that are derivatives of the hemiasterlin analog HTI-286 of Formula I, supra, that additionally comprise a photoreactive group and/or in which a photoreactive group is optionally substituted for one or more of the substituent moieties in Formula I. Exemplary photoreactive groups include an azide and a benzephenone moiety, with the benzephonone moiety being particularly preferred.

For example, compounds of the invention that have the chemical structure set forth in Formula IV, below, are particularly preferred:

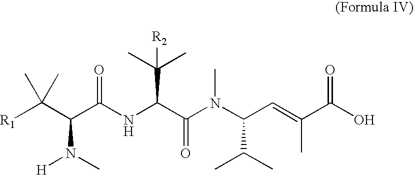

In Formula IV, above, the substituents R₁ and R₂ are independently selected provided that at least one of the substituents R₁ and R₂ comprises a photoreactive moiety such as a benophenone or azide moiety. R₁ can be a photoreactive moiety or an aryle moiety such as a benzene ring. R₂ can be a photoreactive moiety, and alkyl moiety or H.

Specific preferred compounds of the invention include the compounds:
4-benzoyl-N,β,β-trimethyl-L-phenylalanyl-N1-[(1S,2E)-3-carboxy-1-isopropylbut-2-enyl]-N1,3-dimethyl-L-valinamide;
N,β,β-trimethyl-L-phenylalanyl-4-benzoyl-N-[(1S,2E)-3-carboxy-1-isopropyl-2-butenyl]-N,β,β-trimethyl-L-phenylalaninamide;
   and pharmaceutically acceptable salts thereof.

Specific preferred compounds of the invention include compounds, referred to here as Probe 1 and Probe 2, respectively, with the formula:

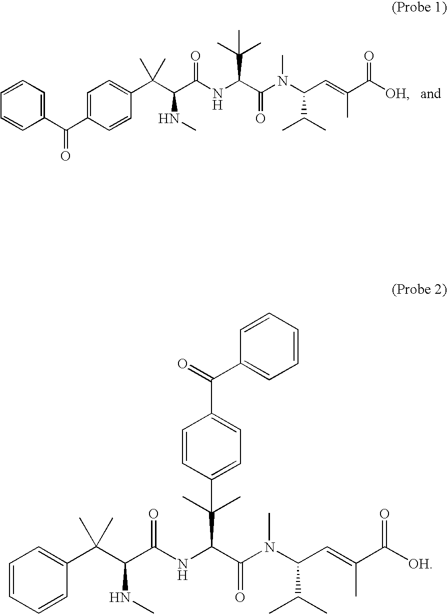

It is understood that, when describing compounds of the present invention, the term alkyl means a saturated linear or branched hydrocarbon moiety of between 1 and 20 carbon atoms, with hydrocarbon moieties of 1 to 10 carbon atoms being preferred. In some embodiments of the invention the alkyl moiety may optionally be 1 to 18 carbon atoms or 1 to 6 carbon atoms. Examples include, but are not limited to, Examples include methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylphenyl, 2,2-dimethylbutyl, n-heptyl, 2-methylhexyl, and the like unless otherwise specified. The carbon atoms may optionally be replaced with 0 to 4 nitrogen atoms, 0 to 4 oxygen atoms, and 0 to 4 sulfur atoms, and the carbon atoms are optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, —SO₂R₁₀, wherein R₁₀ is a an alkyl moiety of 1 to 10 carbon atoms, alkenyl moiety of 2 to 10 carbon atoms, aryl and a cyclic hydrocarbon moiety of 3 to 10 carbon atoms, aryl-R— and heteroaryl-R.

The term alkenyl refers to an unsaturated linear or branched hydrocarbon moiety, preferably between about 2 to 10 carbon atoms and containing at least on carbon-carbon double bond. Each double bond in an alkenyl moiety is independently a cis, trans or nongeometric isomer wherein carbon atoms may optionally be replaced with 0 to 4 nitrogen atoms, 0 to 4 oxygen atoms and 0 to 4 sulfur atoms. In additionally, the carbon atoms are optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, —SO₂R₁₀, wherein R₁₀ is a an alkyl moiety of 1 to 10 carbon atoms, alkenyl moiety of 2 to 10 carbon atoms, aryl and a cyclic hydrocarbon moiety of 3 to 10 carbon atoms, aryl-R— and heteroaryl-R.

The term cyclic hydrocarbon moiety refers to a saturated or unsaturated cyclic hydrocarbon moiety (preferably of 3 to 10 carbon atoms) as well as to a monocyclic cycloalkyl or cyclalkenyl ring of 3 to 10 carbon atoms. Carbon atoms in a cyclic hydrocarbon moiety may optionally be replaced with 0 to 4 nitrogen atoms, 0 to 4 oxygen atoms and 0 to 4 sulfur atoms. In additional, the carbon atoms are optionally substituted with: ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, —SO₂R₁₀, wherein R₁₀ is a an alkyl moiety of 1 to 10 carbon atoms, alkenyl moiety of 2 to 10 carbon atoms, aryl and a cyclic hydrocarbon moiety of 3 to 10 carbon atoms, aryl-R— and heteroaryl-R. In some embodiments of the invention the cyclic hydrocarbon may optionally be a 5 to 14-membered monocyclic, bicyclic or tricyclic saturated or unsaturated hydrocarbon ring moiety wherein the carbon atoms may optionally be replaced with 0 to 4 nitrogen atoms, 0 to 4 oxygen atoms and 0 to 4 sulfur atoms, wherein the carbon atoms may optionally be substituted with R, ═O, ═S, OH, —OR₁₀, —O₂CR₁₀, —SH, —SR₁₀, —SOCR₁₀, —NH₂, —NR₁₀H, —N(R₁₀)₂, —NHCOR₁₀, —NR₁₀COR₁₀, —I, Br, —Cl, —F, —CN, —CO₂H, —CHO, —COR₁₀, —CONH₂, —CONHR₁₀, —CON(R₁₀)₂, —COSH, —COSR₁₀, —NO₂, —SO₃H, —SOR₁₀, or —SO₂R₁₀ where R₁₀ is as defined above.

The term aryl refers to an aromatic hydrocarbon moiety having 6, 10 or 14 carbon atoms and preferably having between 6 and 10 carbon atoms optionally substituted with R, X or Z (as defined supra).

The term heteroaryl refers to a 5- or 6-membered heterocyclic ring, which can be fused to another 5- or 6-membered heterocyclic ring, especially heteraromatic rings that contain 1 to 3 heteroatoms selected which can be, e.g., O, N or S optionally substituted with R or X or fused to a cyclic hydrocarbon moiety of 3 to 10 carbon atoms. Examples of preferred heteroaromatic rings include but are not limited to: thienyl, furyl, indolyl, pyrrolyl, thiophenyl, benzofuryl, benzothiophenyl, quinolyl, isoquinolyl, imidazolyl, thiazolyl, oxazolyl and pyridyl.

The term alkoxy refers to an alkyl-O— group in which the alkyl group is as defined above. Exemplary alkoxy groups include but are not limited to methoxy, ethoxy, n-propoxy, 1-propoxy, n-butoxy and t-butoxy.

Aralkyl refers to an aryl-alkyl group in which the aryl and alkyl group are as defined above. Non-limiting examples of aralkyl groups include benzyl and phenethyl.

The term phenyl refers to a 6-membered carbon aromatic ring.

Preferably, the recitation of a compound of the invention (e.g., a compound of Formula II or III as defined, supra) includes all possible salts of the compound, and also denotes all possible isomers possible within the structural formula specified for such compound, including geometrical and optical isomers. Unless otherwise stated, materials described herein comprising a compound for which isomers exist are to be regarded as covering individual isomers as well as mixtures of isomers, including racemic mixtures.

It is, moreover, to be understood that compounds of the invention may comprise on or more detectable labels, including one or more radiosotopes such as ³H (i.e., “tritium), ¹³C, ³²P or ³⁵S to name a few. For instance, the Examples infra describe embodiments in which the benzephenone moiety in compounds of the invention comprises ³H rather than normal ¹H.

6.2. Uses of Affinity Probes

6.2.1 Identification of Tubulin Binding Sites

Affinity probes of the present invention are particularly well suited for identifying the binding site or sites for anti-tubulin compounds and, in particular, for identifying the binding site or sites of hemiasterlin compounds (including hemiasterlin derivatives and analogs). For instance, the Examples, infra, describe experiments that use affinity probes of the invention to identify hemiaterlin binding sites on tubulin. These experiments involve routine experimental techniques such as enzymatic digestion (for example, digestion using Lys C, Trypsin and/or subtilisin) as well as chemical digestion (e.g., by formic acid and/or CNBr) to digest tubulin after irreversible binding to an affinity probe of the invention. For instance, in the Examples infra, a photoreactive group on the affinity probe is activated (e.g., by UV-irradiation) after incubation with tubulin such that the reactive group irreversibly binds to the tubulin. Digestion fragments of tubulin can be subsequently analyzed to determine which fragment or fragments have an affinity probe bound thereto. The binding site or sites of the affinity probe (and hence of hemiasterlins) is then identified as the site or sites corresponding to the fragment or fragments with an affinity probe bound thereto.

As an example and not by way of limitation, digestion fragments can be separated (e.g., by electrophoretic gels or other chromatography techniques known in the art) and a detectable label (for example, a radiolabel such as ³H) on the affinity probe(s) can be detected. Alternatively, digestion fragments can be analyzed by mass spectroscopy to determine which fragment or fragments have an affinity probe bound thereto.

The Examples, infra, also identify particular binding sites on tubulin for anti-tubulin compounds and, in particular, for hemiasterlin compounds (including their derivatives and analogs). In particular, the results presented in these Examples demonstrate that hemiasterlins bind α-tubulin in a region or regions between about amino acid residues 200 and 350 of α-tubulin and, more preferably, between about amino acid residues 200-340.

In particular, the results in the Examples describe one embodiment (using the affinity probe referred to here as Probe 1) in which hemiasterlins bind to amino acid residues corresponding to a domain referred to as loop 8, helix 10 in α-tubulin. In particular, this domain corresponds to a region comprising amino acid residues 300-350 of α-tubulin. More preferably, this region comprises amino acid residues 310-340, and still more preferably comprises amino acid residues 314-338 of α-tubulin. The Examples also describe another embodiment (using the affinity probe referred to here as Probe 2) in which hemiasterlins bind to a region comprising about amino acid residues 200-300 of α-tubulin, and more preferably comprising amino acid residues 204-280 of α-tubulin. Those skilled in the art appreciate that these portions of the α-tubulin amino acid sequences correspond to domains commonly referred to as helices 6-8, loop 7 and the N-terminal portion of the M-loop of α-tubulin. It is understood, therefore, that hemiasterlins (including hemiasterlin derivatives and analogs such as HTI-286) can be expected to bind to either one or both of these regions on α-tubulin.

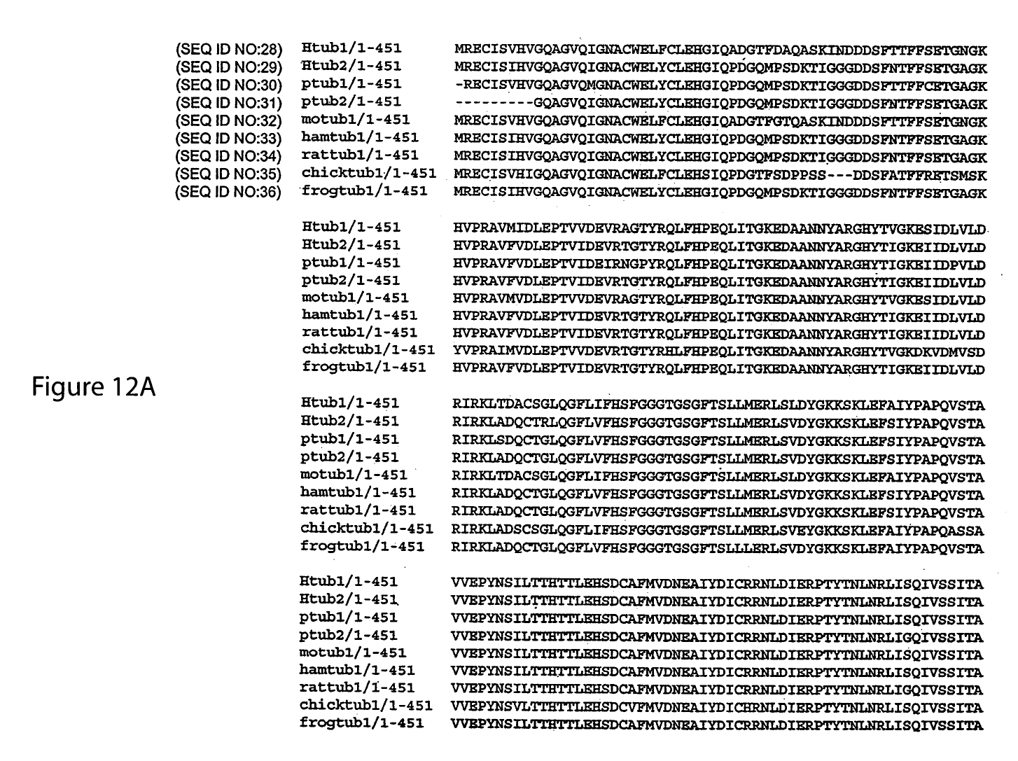

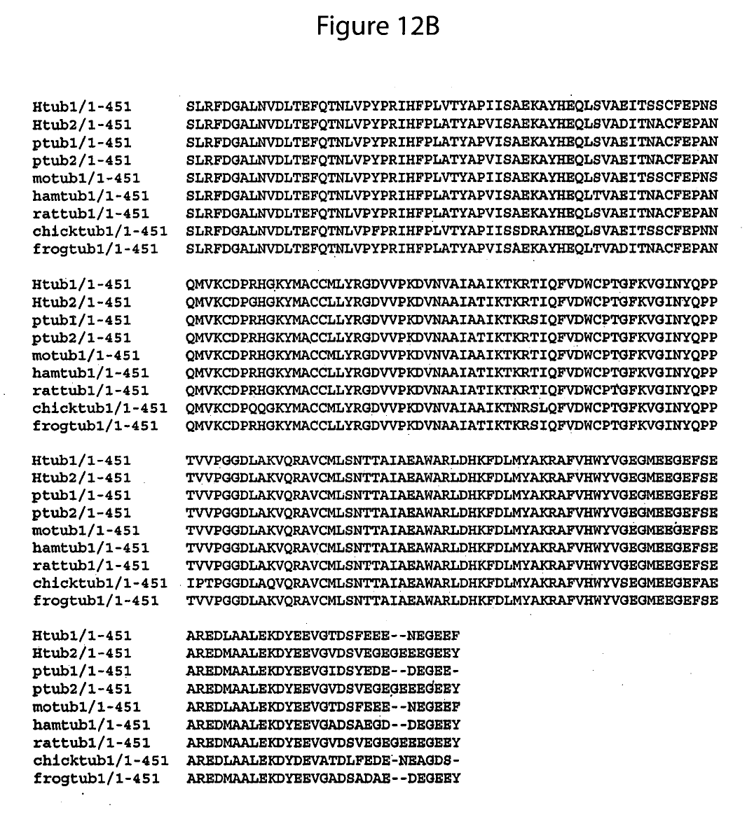

It is well known that the sequence of α-tubulin is highly conserved among different species of organisms and, in particular, among different species of vertebrates. See, in particular, the sequence alignment at FIG. 12. Hence, while the experiments described in the Examples, infra, are done using bovine tubulin, it is understood that the results (including the binding domains and regions described above) are equally applicable to tubulin from other species of organisms, including tubulin from other vertebrate species such as those identified in FIG. 12. Accordingly, the amino acid residues recited above and throughout this specification, unless otherwise noted, specifically refer to amino acid residues in the sequence of human α-tubulin set forth in FIG. 12 However, those skilled in the art will be able to readily identify corresponding domains and regions of other α-tubulin amino acid sequences (including α-tubulin sequences from other species of organisms, orthologs and homologs). For instance, residues of other α-tubulin sequences shown in FIG. 12 that align with the amino acid sequences recited above are also understood to represent binding domains for hemiasterlin compounds.

Those skilled in the art can also readily align different α-tubulin or other amino acid sequences, using routine algorithms such as FASTA (Pearson & Lipman, Proc. Natl. Acad. U.S.A. 1988, 85:2444-2448; Pearson, Methods Enzymol. 1990, 183:63-98), BLAST (Altschul et al., Nucl. Acids Res. 1997, 25:3389-3402; Altschul, J. Mol. Evol. 1993, 36:290-300; Altschul et al., J. Mol. Biol. 1990, 215:403-410), CLUSTAL and CLUSTALW (Higgins et al., Nucl. Acids. Res. 1994, 22:4673-4680), to name a few. Generally, such alignment algorithms will be used with the standard or default parameters, including standard alignment scoring systems and/or a scoring matrix such as BLOSUM62. See, Henikoff & Henikoff, Proc. Natl. Acad. Sci. 1992, 89:10915-10919. However, in certain circumstances that will be appreciated by those skilled in the art, it may be preferable to us nonstandard parameters and/or scoring matrices. For example, in embodiments where very similar amino acid sequences are being compared (such as sequences of α-tubulin) it may be preferably to use a scoring matrix such as BLOSUM90, that has higher cutoffs.

6.2.2 Assays for Characterizing Anti-Tubulin Compounds

Affinity probes of this invention can also be used to characterize compounds based on their ability to inhibit affinity probe binding and/or covalent labeling to tubulin. For example, if a compound significantly inhibits binding of an affinity probe, this compound can be characterized as being similar in structure, mechanism of action, and/or the site of binding compared to the probe. Alternatively, if a compound does not inhibit the binding of an affinity probe, this compound can be characterized as being different in structure, mechanism of action, and/or the site of binding compared to the probe. Compounds characterized according to such methods can possess inhibiting activity ranging anywhere from completely inhibiting an affinity probe from binding tubulin (i.e., complete inhibition) to completely allowing the affinity probe to bind (i.e., no inhibition). Compounds characterized according to these methods can also be partial inhibitors that, while not completely inhibiting binding of the affinity probe to tubulin, nevertheless do inhibit binding to some measurable extent.

Classes of compounds that may be used to inhibit probe binding include, but are not limited to, small molecules (e.g., organic or inorganic molecules which are less than about 2 kDa in molecular weight, are more preferably less than about 1 kDa in molecular weight, and/or are able to cross the blood-brain barrier and affect tubulin or activities associated therewith) as well as macromolecules (e.g., molecules greater than about 2 kDa in molecular weight). Compounds used to inhibit binding may also include peptides and polypeptides. Examples of such compounds (including peptides) include but are not limited to: soluble peptides; fusion peptide members of combinatorial libraries (such as ones described by Lam et al., Nature 1991, 354:82-84; and by Houghten et al., Nature 1991, 354:84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); antibodies, including but not limited to polyclonal, monoclonal, humanized, anti-idiotypic, chimeric or single chain antibodies; antibody fragments, including but not limited to Fab, F(ab′)₂, Fab expression library fragments, and epitope-binding fragments thereof.

General methods for determining in tubulin the inhibition of probe labeling by compounds are described here, and specific, non-limiting examples of these methods are demonstrated in the Examples, infra (see Sections 7.4.2 and 7.4.3). In general, a tubulin-containing sample (preferably comprising at least α-tubulin) is incubated in the presence of both a test compound and a photoaffinity probe of the invention. In preferred embodiments, the tubulin sample is incubated first with the test compound, and the affinity probe is added to that sample after a time sufficient for the test compound to bind tubulin. However, the test compound and affinity probe can also be contacted to the tubulin sample concurrently, and incubated together for a time period sufficient for both compounds to bind. In preferred embodiments, the affinity probe is then covalently bound to the tubulin (for example, by irradiation the sample with UV light) to facilitate detection of the bound affinity probe. The tubulin sample can then be analyzed by routine methods, to determine whether and to what extent the affinity probe is bound to tubulin in the sample. Preferably, these results are compared to data from a control experiment, in which an affinity probe is incubated with the tubulin sample in the absence of a test compound.

Examples of assays that can be used to detect the affinity probe include, but are not limited to, detection of the affinity probe by radioactivity (e.g., where a radiolabeled affinity probe is used) or fluorescence (e.g., where a fluorescently labeled affinity probe is used). Alternatively, the affinity probe may also be detected by other methods such as mass spectroscopy or in an immunoassay (e.g., with an antibody that specifically binds to the affinity probe or, alternatively, specifically binds to tubulin when an affinity probe is bound thereto).

The radioactivity of a probe can be detected by fluorography or scintillation counting as described in the Examples, infra. Probe fluorescence can be detected using methods well known in the art, such as fluorescence microscopy or fluorimetry.

In the use of antibodies, detecting the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, precipitation reactions, agglutination assays (e.g., gel agglutination assays, hemagglutination assays), complement fixation assays, immunofluorescence assays, protein A assays, and immunoelectrophoresis assays, etc.

In one embodiment, antibody binding is assayed by detecting a label on the primary antibody. In another embodiment, the primary antibody is detected by assaying the binding of a secondary antibody or reagent to the primary antibody. In a further embodiment, the secondary antibody is labeled. Many means are known in the art for detecting binding in an immunoassay and are within the scope of the present invention.

As demonstrated in Section 7.4.3, one preferred embodiment of this invention uses a radiolabeled photoaffinity hemiasterlin analog probe. In this example, known tubulin binding drugs, dolastatin-10, vinblastine, paclitaxel, and colchicine, are used as competitive inhibitors of probe binding to tubulin. However, it is understand that other compounds, which may or may not be known tubulin binding drugs, can also be characterized using such methods. In this Example, each test compound is preferably incubated with tubulin followed by the addition of probe. The probe can then be covalently attached to tubulin using UV light. Probe binding to tubulin is assayed, e.g., by separating the samples on an SDS-PAGE gel, slicing the gel, and determining the radioactivity of each gel slice using scintillation counting. The amount of probe radioactivity in tubulin for each inhibition experiment is compared to probe radioactivity in tubulin in the absence of competitor. Using this method, dolastatin-10 and vinblastine inhibited probe labeling to a greater degree and, hence, are more closely related to the probe than paclitaxel or colchicine.

Results from such assays, which evaluate the ability of a test compound to inhibit hemiasterlin probes from labeling tubulin can also be compared to similar assays that use affinity probes for other classes of tubulin binding drugs. Such probes can mimic the binding of, for example, different classes of tubulin-binding drugs such as colchicine, vinca alkaloids, or taxanes. Based on the results of these inhibition studies compounds can be categorized. A compound, for example, can be considered most related in structure, mechanism of action, and/or site of binding to a particular probe if the degree of binding inhibition is higher than for other probes. Preferably, however, test compounds will show probe-labeling inhibition to at least one, all, none, or some (e.g. to varying degrees) of the probes.

6.2.3 Diagnostic and Prognostic Assays

Affinity probes of this invention are also useful for diagnostic and/or prognostic assays, e.g., to identify cancerous cells and tissues both in vitro (such as in cell or tissue cultures) and in vivo. In particular, it is understood that tubulin binding compounds, including affinity probes of this invention, preferably target and bind to tubulin in cancerous cells and tissues which are rapidly dividing. Hence, such cells and tissues can be readily detected, by detecting binding of the affinity probes to tubulin in cells and tissue. Examples of tumors that can be detected according to the invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In other embodiments, affinity probes of the invention can also be used to identify cells and tumors to which a hemiasterlin compound (including hemiasterlin derivatives and analogs such as HTI-286) will bind, and which are therefore susceptible to treatment with those compounds. In other words, affinity probes of the invention can also be used to determine whether a particular cancer cell or tumor (for example, a cell or tumor obtained from a patient biopsy) are or will be resistant to treatment with hemiasterlin compounds such as HTI-286. Such assays are particularly useful, e.g., for identifying therapeutic regimens that are effective for treating a particular cancer and/or for a particular patient.

Cells and/or tissue samples that are tested in such applications can be readily obtained—for example, by obtaining a biopsy sample from a patient who has or is suspected of having a cancer or tumor. Cells and tissue samples can also be maintained in culture, using routine culturing techniques, for later testing according to these methods.

The detection of tumors (including hemiasterlin susceptible tumors) in such assays is based on the the high binding affinity of the affinity probes to tubulin in such cell or tissue samples. Hence, high binding affinity of an affinity probe to cells or tissues is indicative that the cells and/or tissues are cancerous and, moreover, that they are susceptible to treatment with a hemiasterlin compound. In preferred embodiments, cells or tissue samples can be exposed to probes by, for example, bathing in media containing the probe. Non-specific binding of the probe to the sample should be avoided by, for example, washing the sample with media without probe several times after specific binding has occurred. Probe specifically bound to tubulin can be detected using a variety of methods including and of the methods described above—e.g., by assaying the radioactivity and/or fluorescence of the probe, mass spectroscopy to determine the location of the probe, and/or antibody detection of the probe. Such antibodies include but are not limited to polyclonal, monoclonal, chimeric, single chain Fab fragments, and Fab expression library. In addition, each of these antibodies may be fluorescently labeled.

The radioactivity of a probe can be detected by fluorography or scintillation counting as described in the Examples, infra. Probe fluorescence can be detected using methods well known in the art, such as fluorescence microscopy or fluorimetry.

In the use of antibodies, detecting the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), precipitation reactions, agglutination assays (e.g., gel agglutination assays, hemagglutination assays), complement fixation assays, immunofluorescence assays, protein A assays, fluorescence activated cell sorting (i.e. FACS), and immunoelectrophoresis assays, etc. In one embodiment, antibody binding is assayed by detecting a label on the primary antibody. In another embodiment, the primary antibody is detected by assaying the binding of a secondary antibody or reagent to the primary antibody. In a further embodiment, the secondary antibody is labeled. Many means are known in the art for detecting binding in an immunoassay and are within the scope of the present invention.

In another embodiment, probes described here may be used to detect (e.g., to diagnose) dysproliferative changes (such as metaplasias and dysplasias) in epithelial tissues such as those in the cervix, esophagus, and lung. Thus, the present invention provides for detection of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79).

Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia. It is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

The methods described herein are not limited to diagnostic applications, but may also be used in prognostic applications, e.g., to monitor the progression of a disease (such as cancer) that is associated with probe binding to tubulin, or to monitor a therapy thereto. Accordingly, prognostic methods of the invention may comprise, in one exemplary embodiment, monitoring probe binding of cells or tissues from an individual during the course of a treatment or therapy (for example, a drug treatment or chemotherapy regime) for cancer or for another disease associated with abnormal tubulin. Similarly, the methods of the invention may also be used to detect and identify diseased cells and tissue (for example, cancerous cells and tissue) during the course of a therapy.

6.3. Hemiasterlin Binding Data and its Uses

Information about binding sites and/or mechanisms of action for hemiasterlins and other anti-tubulin drugs is useful inter alia for identifying new tubulin binding compounds, including the identification of new hemiasterline derivatives and analogs, e.g., by molecular modeling and/or structure activity relationship (SAR) studies. Hence, the present invention provides methods for identifying new anti-tubulin drugs (particularly, new peptide-containing agents, such as hemiasterlins) which use SAR and/or molecular modeling to identify compounds that bind to one or more binding sites identified by an affinity probe of the invention. Information about binding sites can also be used to identify amino acid sequences and mutations (including tubulin amino acid sequences and mutations) that affect tubulin binding. Such mutations can give rise, e.g., to tumor cells and/or cell lines that are resistant to certain hemiasterlins and other anti-tubulin compounds. Thus, the invention also provides methods for identifying such amino acid sequences, as well as diagnostic and prognostic methods for identifying cells that are resistant to (or are likely to be resistant to) certain hemiasterlins and/or other anti-tubulin compounds. These and other methods of the invention are described in detail herebelow.

6.3.1 Identifying New Tubulin Binding Compounds

Information about binding sites and/or mechanisms of action for hemiasterlins can be used, e.g., in molecular modeling and/or structure activity relationship (SAR) studies to identify new anti-tubulin drugs and, in particular, to identify new hemiasterlin compounds (including new hemiasterlin derivatives and analogs). Such methods, which use information about binding sites obtained from affinity probes (see, the Examples infra) are therefore provided in the present invention.

For example, the present invention provides methods that use computer modeling algorithms and other techniques known in the art to identify compounds that bind (or are expected to bind) a hemiasterlin binding site identified by an affinity probe of this invention—such as one of the binding sites described above. Such methods generally use a three-dimensional or tertiary structure for tubulin or another drug target to model the binding site or sites of candidate compounds. The three dimensional structure of α-tubulin, for example, has been determined and can be obtained, e.g., from the Protein Data Bank (Bennan et al., Nucl. Acids Res. 2000, 28:235-242) under the Accession Nos. 1TUB, 1JFF, and 1FFX. See also, Nogales et al., Nature 1998, 391:199; Lowe et al., J. Mol. Biol. 2001, 313:1045; and Gigart et al., Cell 2000, 102:809.

Because α-tubulin is highly conserved across different species of organisms (see, in particular, the alignment at FIG. 12) the three dimensional structure of tubulin from one species of organism can be readily used to obtain a corresponding three-dimensional structure for tubulin from a different species of organism. In particular, it is understood that the three-dimensional structure of all α-tubulin molecules will be substantially similar regardless of the source or species of organism from which it is derived. In general, two three-dimensional structures are said to be substantially structurally similar to each other if their atomic coordinates have a root-mean square deviation (RMSD) less than or equal to about 1 angstrom, as calculated, e.g., using the Molecular Similarity Module within the QUANT program (QUANTA, available from Molecular Simulations Inc., Sand Diego, Calif.).

Using routine computer modeling algorithms and other techniques that are well known in the art, interactions (e.g., hydrogen bonding, hydrophobic, and/or electrostatic interactions) between hemiasterlin analogs of this invention and tubulin can be identified. Furthermore using these modeling techniques, a user may identify other compounds that are expected to bind to tubulin using similar interactions as the compounds described in this invention. More specifically, using the crystal structure of tubulin, those skilled in the art can identify compounds that bind by forming stabilizing interactions with tubulin, similar to the stabilizing interactions for hemiasterlin. Compounds identified using these modeling techniques can be expected to compete with hemiasterlin analogs for binding to tubulin.

Techniques and algorithms for such computer assisted molecular modeling are well known in the art. Exemplary programs that can be used in such methods include, but are not limited to: AutoDock (Morris et al., J. Computational Chem. 1998, 19:1639-1662), FTDock (Katchelski-Katzin, Proc. Natl. Acad. Sci. 1992, 89:2195-2199; Gabb et al., J. Mol. Biol. 1997, 272:106-120), GROMOS™ (van Gunstener & Berendsen, Agnew. Chem. Int. Ed. Engl. 1990, 29:992-1023) and ICM (available from MolSoft LLC, San Diego Calif.) to name a few.

Classes of compounds that may be identified by such modeling techniques include, but are not limited to, small molecules (e.g., organic or inorganic molecules which are less than about 2 kDa in molecular weight, are more preferably less than about 1 kDa in molecular weight, and/or are able to cross the blood-brain barrier and affect tubulin or activities associated therewith) as well as macromolecules (e.g., molecules greater than about 2 kDa in molecular weight). Compounds identified by these modeling techniques may also include peptides and polypeptides. Examples of such compounds (including peptides) include but are not limited to: soluble peptides; fusion peptide members of combinatorial libraries (such as ones described by Lam et al., Nature 1991, 354:82-84; and by Houghten et al., Nature 1991, 354:84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); antibodies, including but not limited to polyclonal, monoclonal, humanized, anti-idiotypic, chimeric or single chain antibodies; antibody fragments, including but not limited to Fab, F(ab′)₂, Fab expression library fragments, and epitope-binding fragments thereof.

In other exemplary embodiments, compounds identified by computer modeling algorithms or other techniques that are well known in the art of this invention may actually have (or may be expected to have) improved binding or stabilizing interactions with tubulin. For example, compounds identified by these methods may form (or be expected to form) stronger and/or more specific hydrogen bonding interactions with tubulin. These compounds can be expected to be more potent in tubulin binding assays and functional assays that test a compounds ability to inhibit tubulin polymerization and cell profileration.

In vitro or cell culture assays may also be used to determine whether a test compound functions as tubulin binder and an anti-microtubule agent. For instance, the Examples, infra, describe binding assays that determine the stability of binding between tubulin and test agents. Additionally, the Examples, infra, provide an assay to determine the ability of a compound to prevent tubulin polymerization in vitro and assay to assess cell proliferation in the presence of a test compound.

6.3.2 Identification of Resistant Cells and Tumors

Information about binding sites and/or mechanisms of action for hemiasterlin compounds can also be used to identify cells and tissues (e.g., tumors) that are either resistant or likely to be resistant to certain anti-tubulin drugs. In particular, such information can be used to identify cells and tissues that may be resistant to treatment with a hemiasterlin compound—including cells and tissues that are either resistant or are likely to be resistant to treatment with a hemiasterlin derivative or analog such as HTI-286.

As an example, and not by way of limitation, the Examples, infra, demonstrate that hemiasterlins bind α-tubulin in a region or regions between about amino acid residues 200 and 350 of α-tubulin, and more preferably between about amino acid residues 200-340.

In particular, the results in the below Examples describe one embodiment (using the affinity probe referred to here as Probe 1) in which hemiasterlins bind to amino acid residues corresponding to a domain referred to as loop 8, helix 10 in α-tubulin. This domain corresponds to a region comprising amino acid residues 300-350 of α-tubulin. More preferably, this region comprises amino acid residues 310-340, and still more preferably comprises amino acid residues 314-338 of α-tubulin. The Examples also describe another embodiment (using the affinity probe referred to here as Probe 2) in which hemiasterlins bind to a region comprising about amino acid residues 200-300 of α-tubulin, and more preferably comprising amino acid residues 204-280 of α-tubulin.

It is understood, therefore, that hemiasterlins (including hemiasterlin derivatives and analogs such as HTI-286) can be expected to bind to either one or both of these regions on α-tubulin. Moreover, mutations in either one or both of these regions are expected to disrupt the binding of such hemiasterlin compounds to tubulin. Consequently, cells having such mutations may be resistant to certain anti-tubulin compounds and, in particular, to hemiasterlin compounds (including hemiasterlin derivatives and analogs such as HTI-286). Hence, it is possible to identify cells (including cancer and tumor cells, such as ones obtained from a biopsy of a patient or other individual) that are either resistant to or are expected to be resistant to treatment with a hemiasterlin compound, by identifying cells that express tubulin having one or more mutations a binding regions identified with an affinity probe. Such methods are therefore encompassed by the present invention.

Generally speaking, to identify such resistant cells and tumors, it is necessary to determine the nucleotide or protein sequence of the tubulin (in particular, α-tubulin) expressed by a cell or tumor. Generally, it will not be necessary to determine the complete amino acid or nucleotide sequence, but is sufficient to determine only the sequence of one or more binding sites identified by an affinity probe, such as one or more of the binding sites described supra. Using the methods described here it is possible to determine the relevant sequence of tubulin nucleic acid or protein in cells (e.g. derived from cell culture) or tissues from an individual, such as in cells or tissues in a sample obtained or derived from an individual subject or patient. These samples can be derived from, for example, a biopsy sample of a patient suspected of having a type of cancer or other disorder associated with tubulin, or suspected of having a propensity for such a cancer or other disorder.

A variety of methods known in the art may be used to detect assay levels of mutated tubulin sequences in a sample. Diagnostic methods for the detection of tubulin nucleic acids in patient samples or in other cell or tissue sources may involve their amplification, e.g., by PCR (see, for example, the experimental embodiment taught in U.S. Pat. No. 4,683,202) followed by nucleotide sequencing of the amplified molecules using techniques that are well known to those of skilled in the art.

Preferably, the nucleotide or amino acid sequence obtained from a cell or tumor is compared to a consensus or wild-type sequence for tubulin, such as one of the α-tubulin sequences depicted in FIG. 12, or a suitable homolog or ortholog thereof to which an affinity probe of the invention binds or is expected to bind, or which is known to be susceptible to treatment with a hemiasterlin compound. In embodiments where the nucleotide sequence (such as a cDNA or mRNA sequence) encoding a tubulin molecule is determined, it is generally preferably to determine the tubulin amino acid sequence that nucleic acid is expected to encode, to determine whether one or more amino acid residues in a binding region is altered or mutated.

As explained above, the amino acid sequence of α-tubulin is highly conserved among a number of different species of organisms and, in particular, among different species of vertebrates, as shown in FIG. 12. Hence, the amino acid residues recited above and throughout this specification, unless otherwise noted, specifically refer to amino acid residues in the sequence of human α-tubulin set forth in FIG. 12. However, those skilled in the art will be able to readily identify corresponding domains and regions of other α-tubulin amino acid sequences, including α-tubulin sequences from other species of organisms, orthologs and homologs. For instance, residues of other α-tubulin sequences shown in FIG. 12 that align with the amino acid sequences recited above are also understood to represent binding domains for hemiasterlin compounds.

Those skilled in the art can also readily align different α-tubulin or other amino acid sequences, using routine algorithms such as FASTA (Pearson & Lipman, Proc. Natl. Acad. U.S.A. 1988, 85:2444-2448; Pearson, Methods Enzymol. 1990, 183:63-98), BLAST (Altschul et al., Nucl. Acids Res. 1997, 25:3389-3402; Altschul, J. Mol. Evol. 1993, 36:290-300; Altschul et al., J. Mol. Biol. 1990, 215:403-410), CLUSTAL and CLUSTALW (Higgins et al., Nucl. Acids. Res. 1994, 22:4673-4680), to name a few. Generally, such alignment algorithms will be used with the standard or default parameters, including standard alignment scoring systems and/or a scoring matrix such as BLOSLUM62. See, Henikoff & Henikoff, Proc. Natl. Acad. Sci. 1992, 89:10915-10919. However, in certain circumstances that will be appreciated by those skilled in the art, it may be preferable to us nonstandard parameters and/or scoring matrices. For example, in embodiments where very similar amino acid sequences are being compared (such as sequences of α-tubulin) it may be preferably to use a scoring matrix such as BLOSUM90, that has higher cutoffs.

Preferred embodiments of such a detection scheme include the use of genomic DNA or the synthesis of a cDNA molecule from an RNA molecule of interest (e.g., by reverse transcription). A sequence within the DNA may then be used as a template for a nucleic acid amplification reaction such as PCR. Nucleic acid reagents used as synthesis intitation reagents (e.g., primers) in the reverse transcription and amplification steps of such an assay are preferably chosen from the tubulin nucleic acid sequences or are fragments thereof. Preferably, the nucleic acid reagents are at least about 9 to 30 nucleotides in length. PCR product can then be sequenced by standard methods to those familiar with the art.

### 7. EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing the invention in spirit or in scope. The invention is therefore to be limited only by the terms of the appended claims along with the full scope of equivalents to which those claims are entitled.

7.1. Materials and Methods

7.1.1 Reagents

HTI-286 (N,β,β-trimethyl-L-phenylalany-N¹[(1S,2E)-3-carboxy-1-isopropylbut-2-enyl]-N¹,3-dimethyl-L-valinamide trifluoroacetate, also known as SPA 110, was synthesized based on methods reported previously (Nieman et al., J. Nat. Prod. 2003, 66:183-199) that were subsequently modified (Zask et al., Proc. Am. Assoc. Cancer Res. 2002, 43:737). Two benzophenone photoaffinity analogs of HTI-286 were made (Kaplan et al., National Medicinal Chemistry Symposium 2002, abstract 49). The first analog, which is referred to here as Probe 1, is the compound 4-benzoyl-N,β,β-trimethyl-L-phenylalanyl-N¹-[(1S,2E)-3-carboxy-1-isopropylbut-2-enyl]-N¹,3-dimethyl-L-valinamide (Kaplan et al., National Medicinal Chemistry Symposium 2002, abstract 49). The second analog, referred to here as Probe 2, is the compound N,β,β-trimethyl-L-phenylalanyl-4-benzoyl-N-[(1S,2E)-3-carboxy-1-isopropyl-2-butenyl]-N,β,β-trimethyl-L-phenylalaninamide. The chemical structure and other properties of these HTI analogs are summarized in Table 1, below.

Except where otherwise stated, experiments are done using microtubule associated protein (MAP)-rich bovine brain tubulin. MAP-rich bovine brain tubulin (99% purity), Hela cell tubulin (90% purity), guanosine-5′-triphosphate (GTP) and PEM buffer (80 mM piperazine-N,N′-bis(2-ethanesulfonic acid) containing 1 mM ethylenebis(oxyethylenenitrilo)tetraacetic acid (EGTA) and, 1 mM magnesium chloride, pH=6.8)) were obtained from Cytoskeleton (Denver, Colo.). Paclitaxel, vinblastine, colchicine, leupeptin, dimethylsulfoxide (DMSO), phenylmethanesulfonyl fluoride (PMSF), sodium dodecyl sulfate (SDS) and subtilisin were obtained from Sigma (St. Louis, Mo.). Dolastatin-10 was obtained from the National Cancer Institute. Probe 1, Probe 2, HTI-286, paclitaxel, vinblastine, colchicine, and dolastatin-10 were prepared as 1 or 10 mM stocks in DMSO for competition studies. Trypsin treated with L-(tosylamido-2-phenyl)ethyl chloromethyl ketone (TPCK) was obtained from Worthington Biochemical Corporation (Freehold, N.J.). Formic acid, cyanogen bromide (CNBr), iodoacetamide (IAM), dithiothreitol (DTT), and trifluoroacetic acid (TFA) were obtained from Pierce (Rockford, Ill.). Guanidine hydrochloride was obtained from the Aldrich Chemical Co., Inc. (Milwaukee, Wis.). Acetic acid, methanol, and acetonitrile were from Fisher Scientific (Fair Lawn, N.J.). Beckman tissue solubilizer-450 (BTS-450) and Ready Protein, a liquid scintillation cocktail, were obtained from Beckman Coulter Instruments, Inc. (Fullerton, Calif.). EN³HANCE autoradiography enhancer was obtained from NEN™ Life Science Products, Inc. (Boston, Mass.). Precast gels used for electrophoresis were obtained from BioRad (Hercules, Calif.).

KB cells are obtained from the American Type Culture Collection (Manassas, Va.) accession no. CCL-17.

7.1.2 Reversible Binding Affinity Assays

The affinity of both HTI-286 and of non-radiolabled Probe 1 and Probe 2, to bovine brain tubulin was determined by previously published methods (Krishnamurthy et al., Biochem., submitted). Briefly, 5 mg/mL rhodamine-labeled tubulin prepared in PEM buffer was pre-incubated with buffer or varying concentrations of test agents in individual quartz cuvettes. The intrinsic tryptophan fluorescence of tubulin (excitation at 560 nm and maximal emission at 580 nm) was measured after pre-incubation for 90 minutes. The change in fluorescence intensity at 580 nm in the presence of compound was fitted to a quadratic equation to obtain apparent dissociation constants (K_{D}) of the test agents.

Binding of Probe 1 to bovine brain tubulin was also assessed by incubating 0.5 μM tubulin with 0.005-5 μM radiolabeled Probe 1 both in the presence and in the absence of 5 μM non-radiolabeled probe 1 as a competitor. Incubations were performed for 30 minutes at room temperature in PEM buffer. Binding was measured using centrifugal gel filtration on Sephadex G-50 (Microspin G-50) as described (Bai et al., Cancer Res. 1996, 56:4398-4406). Spin columns (Amersham Biosciences, Piscataway, N.J.) were centrifugated for 1 minute at 2000 rpm in a microcentrifuge immediately prior to use, and samples applied in 50 μL total volume, followed by a 2 minute centrifugation at the same speed. The amount of probe 1 bound to tubulin was quantitated by measuring the radioactivity within aliquots collected after centrifugation. The K_{D} value, representing 50% of specific binding, was calculated from the curve generated by subtracting the amount of radiolabeled probe bound in the absence of probe from that bound in the presence of non-radiolabeled probe.

7.1.3 Tubulin Polymerization Assay in a Cell-Free System

In vitro tubulin polymerization assays were performed according to procedures that have been described elsewhere (Loganzo et al., Cancer Res. 2003, 63:1838-1845). Briefly, bovine MAP-rich tubulin (final concentration 1.5 mg/mL) was dissolved in cold PEM buffer containing 1 mM GTP (GPEM) and centrifuged at 12,000×g for 10 minutes at 4° C. The tubulin solution (100 μL/well) was added rapidly to wells of a low-volume, 96-well plate already containing duplicate aliquots (10 μL) of test compounds in GPEM. Final compound concentrations were 0.3 μM. Control wells contained the same final concentration of DMSO (0.3%). After initiation of the reaction, absorbance at 340 nm was measured every minute for 60 minutes at 24° C. using a SpectraMax Plus plate reader (Molecular Devices, Sunnyvale, Calif.). An increase in absorbance over time indicated an increase in turbidity resulting from tubulin polymerization.

7.1.4 Cell Proliferation Assay in Tissue Culture

Cell proliferation studies were performed according to procedures described by Loganzo et al. (Cancer Res. 2003, 63:1838-1845). Briefly, KB cells were plated in 96-well plates in 100 μL media at densities pre-determined to produce 60-90% confluence at the time of analysis. Compounds, which were serially diluted into media as 2× stocks, were added to cells in duplicate. After 72 hours of incubation, cell survival was assayed by the SRB assay as described (Rabindran et al., C. Cancer Res. 1998, 58:5850-5858). IC₅₀ values (i.e. the concentration of drug needed to inhibit cell growth by 50%) were used to evaluate drug potency.

7.1.5 Photolabeling, SDS-PAGE and Fluorography Techniques

Photoaffinity labeling studies were done by incubating tubulin (2.5 μM and 5 μM for Probe 1 and 2 experiments, respectively, unless otherwise noted) prepared in PEM buffer with or without competitor molecule for 15 minutes at 4° C., room temperature, or 37° C., prior to incubation with radiolabeled probe (2.5 or 0.25 μM) at room temperature or 37° C. for 30 minutes. Alternatively, a cytosolic preparation of KB-3-1 cell lysates (50 μg) or KB-3-1 cells (40,000 cells) were used in the place of purified tubulin above. The supernatant of mechanically disrupted cells centrifuged at 100,000×g was used as a cytosolic preparation. K-3-1 cells were prepared by growing KB-3-1 cells in a 96-well plate and rinsing them with serum free medium. For experiments with whole cells, 250 μM HTI-286 was used as the competitor molecule during pre-incubation at 37° C. for 15 minutes.

Samples were irradiated at 360 nm with a Mineralight lamp (UVP, Upland, Calif.) for 30 minutes (for Probe 1), 2 hours (for whole cells with Probe 1), or 2 hours (for Probe 2) at 4° C., unless otherwise noted. Radiation of the benzophenone moiety at this wavelength effectively activates the photoprobe with minimal destruction of protein compared to the shorter wavelengths typically used for azido-containing photoprobes (see, Williams et al., Methods Enzymol. 1986, 126:667-682). If whole cells were used, Laemmli sample buffer was then added to lyse the cells.

Photolabeled samples were analyzed by SDS-PAGE using 7.5% Tris-HCl gels. Low grade SDS (Sigma, catalog No. L5750) was used in the running buffer, when needed, to allow separation of α and β-tubulin subunits. Gels were fixed, stained with Coomassie blue from BioRad (Hercules, Calif.) to ensure equal loading of protein, and sliced. The radioactivity of each 1 mm slice was determined by placing the slice overnight at room temperature in 200 μL of 90% BTS-450, followed by the addition of 6 mL Ecolume scintillation cocktail (for Probe 1) or Ready Protein scintillation cocktail (for Probe 2) before subjecting the vials to scintillation counting. Alternatively, gels were incubated with EN³HANCE according to the manufacturer's instructions (Life Sciences Products, Inc.), washed, and dried prior to exposure of the gel to Biomax MX film from Kodak (Rochester, N.Y.) for fluorography.

7.1.6 Formic Acid Digestion after Photoaffinity Labeling Using Probe 1

50 μM tubulin, which was photolabeled with 5 μM (7.2 μCi) [³H] Probe 1 according to methods supra, was run on a 7.5% Tris-HCl SDS-PAGE gel that allowed the α and β-tubulin to co-migrate. Tubulin was excised from the gel by cutting the unstained gel in the region that co-migrated precisely with the 50 kDa marker of the Precision Protein Standards (BioRad). The material was digested in-gel in 250 μL of 75% formic acid at 37° C. After 44-72 hours, formic acid was removed in a speed vacuum, and digestion products were separated by SDS-PAGE on Tris-tricine gels (10-20%). Radiolabeled peptides were visualized by fluorography. In other types of experiment, tubulin subunits were separated on 7.5% Tris-HCl gels (BioRad) and α- and β-tubulin bands were cut and digested separately. The sequence of the labeled formic acid digestion fragment was confirmed by mass spectrometric (MS) analysis as described below.

7.1.7 Trypsin Digestion of Native Tubulin after Photoaffinity Labeling

Native tubulin (15 μg for Probe 1 or 5 μg for Probe 2) was incubated with 0.25 μM (0.35 μCi) of [³H]-Probe 1 or 2.5 μM [³H]-probe 2 in PEM buffer at room temperature for 30 minutes. Samples were then irradiated at 4° C. for 2 hours and digested with 0.8 μg trypsin for 5 to 60 minutes at 30° C. The reaction was stopped by adding leupeptin to 0.01 mM. The samples were separated on 10-20% Tris-tricine gels. The radioactivity within 1 mm gel slices was determined. Alternatively, peptides were resolved on 7.5% Tris-HCl and gels were exposed to film using fluorographic methods described above.

7.1.8 CNBR Cleavage after Photoaffinity Labeling of Tubulin

Tubulin (2.5 μM) was photolabeled with 0.25 μM [³H]-Probe 1 or 2.5 μM [³H]-Probe 2 as described above. α- and β-isomers were then resolved on a 7.5% Tris-Glycine SDS gel for Probe 1 or a 7.5% Tris-HCl SDS gel for Probe 2. Gel bands were independently excised, reduced with dithiothreitol (10 mM in 100 mM ammonium bicarbonate) and alkylated with iodoacetamide (25 mM in 100 mM ammonium bicarbonate).

The gel bands containing separated tubulins were shrunk with 100% acetonitrile and reswelled with CNBr (150 mg/ml) in 70% formic acid. Digestion was allowed to proceed for 4.5 hours at room temperature. Gel bands were then evaporated to dryness from H₂O several times in vacuum to remove excess reagents. The gel bands were then reswollen with SDS loading buffer and mounted on top of a 10-20% Tris-tricine gel where digestion products were separated. Replicate gels were run of the CNBr fragments: one gel was electroblotted to PVDF for autoradiography and the other was silver stained (Shevchenko et al., Analytical Chemistry 1996, 68:850-858). Blots for autoradiography were first soaked with a solution of PPO in toluene and autoradiographic images were captured on Kodak BioMax MS film.

As in the formic acid digestion protocol (see Section 7.1.6), the sequence of the labeled CNBr digestion fragment was confirmed by mass spectrometric analysis from the silver stained gel as described below.

7.1.9 Mass Spectrometric Analyses of Digestion Fragments

Peptides from the in-gel enzymatic digestion were injected onto a self-packed PicoFrit C18 column from New Objectives (Woburn, Mass.) that is directly interfaced to an LCQ Deca ion trap mass spectrometer from ThermoFinnigan (San Jose, Calif.). During a 90 minute HPLC gradient [4% to 60% solvent B (0.1 M acetic acid/90% ACN/H20), solvent A: 0.1 M acetic acid/H20], the mass spectrometer was operated in a data-dependent mode using software provided by the manufacturer to acquire both MS (peptide mass) and MS/MS (fragment ion mass) spectra. Peptide sequences were determined by matching the fragment ion spectra against the non-redundant NCBI protein database using the Sequest search algorithm provided by ThermoFinnigan.

7.1.10 Subtilisin Digestion of Tubulin Before or after Labeling with Probe 1

Before or after incubating with 0.25 μM (0.36 μCi) [³H]-Probe 1 for 30 minutes at room temperature and irradiating as described in Section 7.1.5, tubulin (5 μg) was digested under native conditions with subtilisin (0.2 μg) for 60 minutes at 30° C. Enzymatic digestions were stopped with 1 mM PMSF. After both digestion and labeling, samples were resolved by SDS-PAGE under conditions that allowed separation of α- and β-tubulin subunits. Gels were then fixed and stained with Coomassie blue from BioRad (Hercules, Calif.) and subjected to fluorography.

7.1.11 Formic Acid and Lys C Digestion of Tubulin after Labeling with Probe 2

Probe 2 photolabeled tubulin (as described in Section 7.1.5) was digested with formic acid (75%) or Lys C (0.1 μg) in the presence of 0.1% SDS at 37° C. for 72 hours. The samples were separated on 10-20% Tris-tricine gels. The gels were exposed to film using fluorographic methods described above.

7.1.12 Subtilisin Digestion of Tubulin before Labeling with Probe 2

Tubulin was digested with 0.2 μg of subtilisin for 60 min at 30° C. and the reaction was stopped by the addition of PMSF (1 mM). Digested tubulin was then photolabeled with Probe 2 as described in Section 7.1.5. The samples were separated on 10-20% Tris-tricine gels. The gels were exposed to film using fluorographic methods described in Section 7.1.5.

7.1.13 Subtilisin Digestion of Tubulin after Incubation of Tubulin with Various Compounds

Tubulin (5 μg) was incubated with 50 μM drug at room temperature for 25 minutes and then digested under native conditions with subtilisin (0.05 μg) for 0, 5, 10, 15, 30 and 60 min at room temperature. Enzymatic digestion was stopped with 1 mM PMSF and samples were resolved by SDS-PAGE under conditions that allowed separation of α- and β-tubulin subunits. Gels were then fixed and stained with Coomassie blue.

7.1.14 Separation of Polymerized and Free Tubulin

Tubulin (5 μg) was incubated with 50 μM drug at room temperature for 25 minutes and then centrifuged at either 10,000 g for 15 minutes in a tabletop microfuge or at 100,000 g for 15 minutes in an airfuge. The supernatant was carefully removed to a fresh tube to obtain free tubulin and the pellet was solubilized in SDS-PAGE sample buffer. The free and polymerized tubulin were run on a 7.5% Tris-HCl gel and gels were stained with Coomassie blue.

7.2. Synthesis of Hemiasterlin Analog Probes

Probes were designed to mimic the binding of hemiasterlin and its analogs. SARs (structure activity relationship) studies on hemiasterlins and their analogs (Nieman et al., J. Nat. Prod. 2003, 66:183-199; Zask et al., Proc Am. Assoc. Cancer Res. 2002, 43:737) have determined which structural modifications in hemiasterlin allow the molecule to retain activity. Probe designs were constrained to only these modifications. More that one probe was designed and synthesized with the intent to define the binding at more than one point of contact. The syntheses and radiolabeling of Probe 1 and Probe 2 are described in the sections, infra.

7.2.1 Synthesis of Probe 1 and Probe 2 Precursors

Scheme I, below, diagrams the synthesis of N-(tert-butoxycarbonyl)-β,β-dimethyl-4-(2-phenyl-1,3-dioxolan-2-yl)-L-phenylalanine (Compound 2) and 4-benzoyl-N-(tert-butoxycarbonyl)-β,β-dimethyl-L-phenylalanine (Compound 8) that are precursors for Probe 1 and Probe 2, respectively.

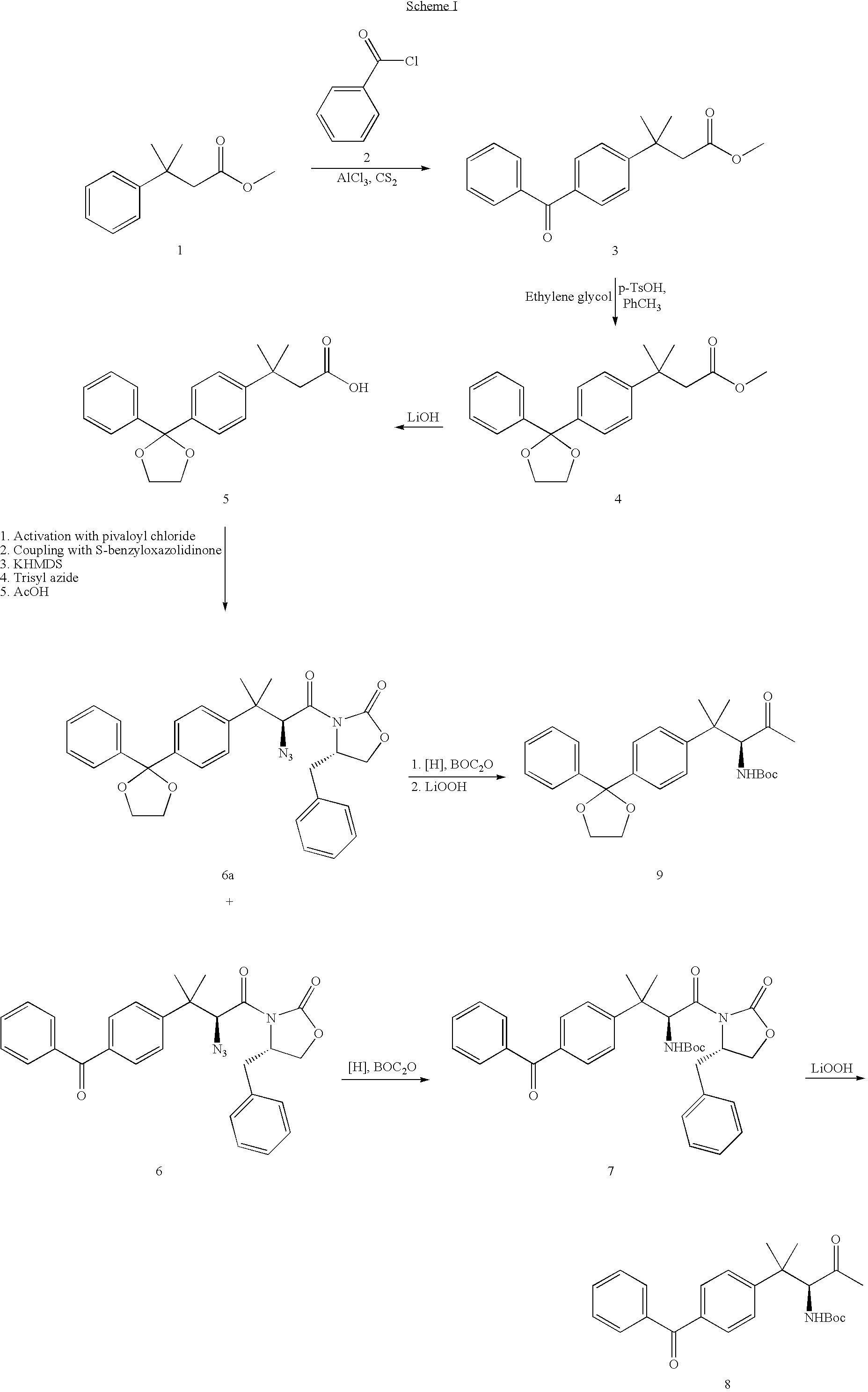

The syntheses, below, describe the preparation of the compounds shown in

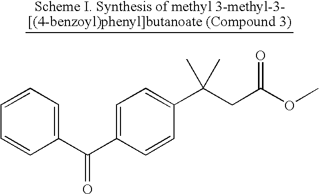

According to Scheme I, a round-bottomed flask is charged with methyl 3-methyl 3-phenyl butanoate (0.25 g, 1.3 mmol, Compound 1), benzoyl chloride (0.15 mL, 1.3 mmol, Compound 2), and carbon disulfide (1.6 mL). While stirring under nitrogen atmosphere, the reaction mixture is cooled to 0° C. in an ice-water bath. Aluminum chloride (0.35 g, 2.6 mmol) is added in a single portion and the cooling bath is removed. The reaction mixture is heated at reflux 4 hours and then allowed to cool to room temperature. The reaction mixture is transferred dropwise into ice-water. The aqueous phase is extracted thrice with dichloromethane. The combined organic extracts are washed with water and 5% aqueous potassium carbonate, dried over sodium sulfate, decanted, and concentrated under reduced pressure to give a brown liquid. MS (ES⁺): m/z (M+H)=297.5

### Synthesis of methyl 3-methyl-3-[4-(2-phenyl-1,3-dioxolan-2-yl)phenyl]butanoate (Compound 4)

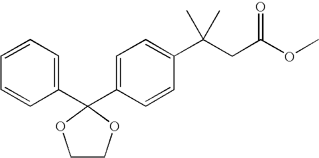

According to Scheme I to a solution of methyl 3-methyl-3-[(4-benzoyl)phenyl]butanoate (26 mmol max, Compound @3 in toluene (100 mL), ethylene glycol (3.2 g, 52 mmol) and p-toluenesulfonic acid monohydrate (˜10 mg) are added. After heating at reflux for 2 hours, an additional 5 mL ethylene glycol and a Dean-Stark trap are added, and reflux is re-started. Azeotropic distillation of water is allowed to proceed overnight. A mixture of ethylene glycol and water (8 mL) is observed in the Dean-Stark trap. The LC/MS of an aliquot of the reaction mixture revealed the presence of both starting material and desired product. The reaction mixture is concentrated under reduced pressure and the residue is partitioned between diethyl ether and saturated aqueous sodium hydrogen carbonate. The aqueous phase is extracted thrice with diethyl ether. The combined extracts are washed with saturated aqueous sodium hydrogen carbonate and saturated aqueous sodium chloride, dried over sodium sulfate, decanted, and concentrated under reduced pressure. The crude product is taken up in toluene (100 mL); and ethylene glycol (3.2 g, 52 mmol) and p-toluenesulfonic acid monohydrate (˜10 mg) are added. The reaction mixture is heated at reflux for 4 hours without the Dean-Stark apparatus and then overnight with the trap. After cooling the reaction mixture to room temperature, the aqueous work-up above is performed. The crude residue is purified by flash chromatography (ethyl acetate/hexanes) to furnish (2.6 g, 29% for 2 steps) of an amorphous white solid. TOF MS (ES⁺): (M+H)=341.3

### Synthesis of 3-methyl-3-[4-(2-phenyl-1,3-dioxolan-2-yl)phenyl]butanoic acid (Compound 5)

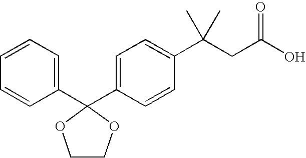

According to Scheme I to a suspension of methyl 3-methyl-3-[4-(2-phenyl-1,3-dioxolan-2-yl)phenyl]butanoate (2.6 g, 7.6 mmol, Compound 4) in tetrahydrofuran (20 mL), methanol (20 mL), and water (10 mL), lithium hydroxide monohydrate (0.48 g, 11 mmol) is added. The mixture is heated at 55° C. for 5 hours, during which all solids dissolved. The reaction mixture is then allowed to cool to room temperature and solvents are evaporated under reduced pressure. The white solid is partitioned between ethyl acetate and water. Most of the material remained undissolved. The biphasic mixture is cooled to 0° C. in an ice-water bath. Glacial acetic acid is added in portions until pH=5. At this point, white solid precipitated, leaving a clear, colorless supernatant. Ethyl acetate is then removed under reduced pressure, and the solids are isolated by filtration of the aqueous phase and washed with cold water. After drying, a white solid is obtained (2.3 g, 92%). TOF MS (ES⁺): (M+H)=327.2

### Synthesis of (4S)-3-{(2S)-2-azido-3-methyl-3-[4-(2-phenyl-1,3-dioxolan-2-yl)phenyl]butanoyl}-4-benzyl-1,3-oxazolidin-2-one (Compound 6a) and (4S)-3-{(2S)-2-azido-3-(4-benzoylphenyl)-3-methylbutanoyl]-4-benzyl-1,3-oxazolidin-2-one (Compound 6)

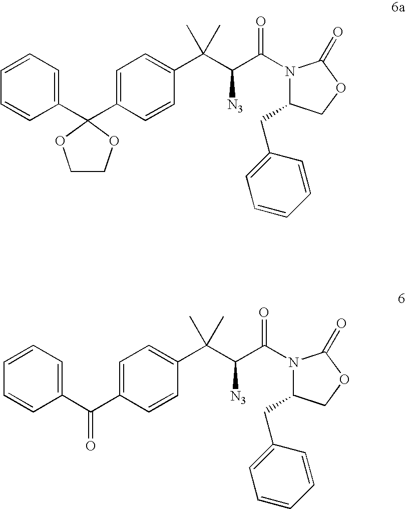

According to Scheme I to a solution of 3-methyl-3-[4-(2-phenyl-1,3-dioxolan-2-yl)phenyl]butanoic acid (2.3 g, 7.0 mmol, Compound 5) in anhydrous tetrahydrofuran (14 mL) under a nitrogen atmosphere, triethylamine (1.2 mL) is added. The mixture is cooled to −78° C. in a dry-ice acetone bath. Pivaloyl chloride (0.91 mL, 7.4 mmol) is added dropwise, causing the immediate formation of a white precipitate. The reaction mixture is allowed to sit for 20 minutes at −78° C. and is then stirred at 0° C. in an ice-water bath. In a separate flask, a solution of S-benzyloxazolidinone (1.2 g, 6.9 mmol) in anhydrous tetrahydrofuran is prepared under a nitrogen atmosphere and cooled to −35° C. in a dry-ice/acetone bath. A small amount of triphenylmethane (<5 mg) is added as an indicator of deprotonation. n-butyllithium (1.6 M solution in hexanes, 4.5 mL, 7.2 mmol) is added dropwise via syringe. At the end of this addition, the characteristic pinkish orange color of the anion of triphenylmethane is not yet observed. However, after the introduction of an additional 0.2 mL of n-butyllithium, this color is achieved. After 30 minutes of stirring at 0° C., the flask containing the mixed anhydride is re-cooled to −78° C. in a dry-ice/acetone bath. The solution of the lithium anion of the oxazolidinone is added to the mixed anhydride solution via a cannula. The source flask is washed twice with tetrahydrofuran (4 mL×2) and these washings are also transferred via a cannula to the mixed anhydride solution. The reaction mixture is stirred at −78° C. for 1 hour and at 0° C. for 1 hour, and then allowed to warm to room temperature overnight. Water (˜15 mL) is added and stirring is continued for 10 minutes. The aqueous phase is extracted thrice with diethyl ether. The combined extracts are washed with saturated aqueous sodium hydrogen carbonate and saturated aqueous sodium chloride, dried over sodium sulfate, decanted, and concentrated under reduced pressure to afford (3.6 g, >100% crude) of a white foam. MS (ES⁺): m/z (+H)=486.2

A solution of this crude benzophenone ketal oxazolidinone (7.0 mmol maximum) in anhydrous tetrahydrofuran (40 mL) is cooled to −78° C. in a dry-ice/acetone bath while stirring under a nitrogen atmosphere. Potassium hexamethylsilazide (0.5 M solution in toluene, 18 mL, 9.0 mmol) is added dropwise to the solution via a syringe. A deep orange-red color resulted from this addition. After stirring for 1 hour at −78° C., a pre-cooled solution of triisopropylsulfonyl azide (3.0 g, 9.8 mmol) in tetrahydrofuran (20 mL) at the same temperature is added rapidly via a cannula. After stirring for 3 minutes at −78° C., the reaction mixture is quenched by the addition of glacial acetic acid (1.8 mL), which caused a color change from deep red to pale yellow. The cooling bath is removed and the reaction is stirred at room temperature for 20 minutes, followed by 1 hour at 40° C. After cooling to room temperature, the reaction mixture is diluted with water and extracted thrice with diethyl ether. The combined extracts are washed with saturated aqueous sodium hydrogen carbonate and saturated aqueous sodium chloride, dried over sodium sulfate, decanted, and concentrated under reduced pressure to afford a pale yellow oil. This oil is inert to hydrolysis of the ketal by two methods: treatment with p-toluenesulfonic acid in aqueous acetone and with aqueous hydrochloric acid in tetrahydrofuran. The unaffected crude material is purified by flash chromatography (hexanes/ethyl acetate) to afford a clean separation of (4S)-3-{(2S)-2-azido-3-methyl-3-[4-(2-phenyl-1,3-dioxolan-2-yl)phenyl]butanoyl}-4-benzyl-1,3-oxazolidin-2-one (0.75 g, 1.4 mmol, Compound 6a) and (4S)-3-{(2S)-2-azido-3-(4-benzoylphenyl)-3-methylbutanoyl]-4-benzyl-1,3-oxazolidin-2-one (0.82 g, 1.7 mmol, Compound 6), giving a total yield (3.1 mmol/7.0 mmol) of 44% for three steps—the formation of the mixed anhydride, displacement with the lithium oxazolidinone, and preparation of the azide. (4S)-3-{(2S)-2-azido-3-methyl-3-[4-(2-phenyl-1,3-dioxolan-2-yl)phenyl]butanoyl}-4-benzyl-1,3-oxazolidin-2-one: TOF MS (ES⁺)=527.4 (4S)-3-{(2S)-2-azido-3-(4-benzoylphenyl)-3-methylbutanoyl]-4-benzyl-1,3-oxazolidin-2-one: TOF MS m/z (ES⁺)=483.4

### Synthesis of (alphaS)-4-Benzoyl-N-{(4S)-3-[4-benzoyl-N-(tert-butoxycarbonyl)-β,β-dimethyl-L-phenylalanyl]-4-benzyl-2-oxo-1,3-oxazolidin-2-yl}-N-{(4S)-3-[4-benzoyl-N-(tert-butoxycarbonyl)-β,β-dimethyl-L-phenylalanyl]-4-benzyl-2-oxo-1,3-oxazolidin-4-yl}-N-(tert-butoxycarbonyl)-β,β-dimethyl-L-phenylalaninamide (Compound 7)

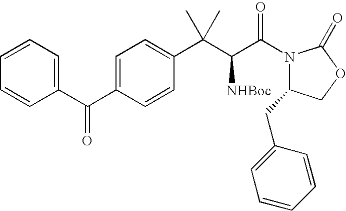

According to Scheme I, a solution of (4S)-3-{(2S)-2-azido-3-(4-benzoylphenyl)-3-methylbutanoyl]-4-benzyl-1,3-oxazolidin-2-one (0.80 g, 1.7 mmol, Compound 6) in ethyl acetate (8 mL) is degassed with a small piece of dry ice. When all effervescence had subsided, palladium on carbon (Pd/C, 10%, 10 mg) is added in a single portion, followed by di-t-butyl dicarbonate (0.74 g, 3.4 mmol). The reaction flask is evacuated under weak house vacuum and then flushed with hydrogen (balloon pressure). This process is repeated thrice. Finally, the reaction mixture is allowed to stir under a hydrogen atmosphere. After 30 minutes, the reaction is incomplete according to thin-layer chromatography (TLC, 20% ethyl acetate/hexanes); hence, stirring under hydrogen is continued over the weekend (˜64 hours). Following this interval, TLC showed a complete disappearance of starting material and the emergence of two new spots. LC/MS analysis revealed these products to be both the desired material and the benzyl phenyl alcohol, the by-product of ketone reduction. The reaction mixture is filtered through a Diatomaceous earth pad to remove Pd/C. The filtrate is concentrated under reduced pressure to afford a clear, colorless oil. This material is subjected to manganese (IV) oxide in dichloromethane in order to oxidize the alcohol by-product back to the benzophenone. This method, however, proved to be very sluggish was aborted. The crude mixture (1.7 mmol max. of alcohol) was then taken up in dichloromethane (10 mL). Pyridinium dichromate (0.96 g, 2.6 mmol) was added to the solution and the resulting rust-colored mixture is stirred overnight at room temperature. TLC showed a complete conversion of by-product to desired product. The reaction mixture is filtered through a Diatomaceous earth pad to remove most of the chromium salts. The filtrate is concentrated under reduced pressure to a dark brown oil, and this crude material is purified by flash chromatography (ethyl acetate/hexanes) to afford (0.52 g, 55%) of a hard, white foam. An additional 0.24 g of slightly impure material is also recovered and removed.

TOF MS m/z (ES⁺)=557.5

### Synthesis of 4-benzoyl-N-(tert-butoxycarbonyl)-β,β-dimethyl-L-phenylalanine (Compound 8)

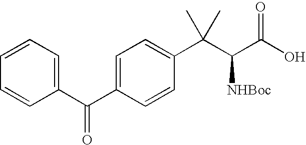

According to Scheme I to a 0° C. solution of (alphaS)-4-benzoyl-N-{(4S)-3-[4-benzoyl-N-(tert-butoxycarbonyl)-β,β-dimethyl-L-phenylalanyl]-4-benzyl-2-oxo-1,3-oxazolidin-2-yl}-N-{(4S)-3-[4-benzoyl-N-(tert-butoxycarbonyl)-β,β-dimethyl-L-phenylalanyl]-4-benzyl-2-oxo-1,3-oxazolidin-4-yl}-N-(tert-butoxycarbonyl)-β,β-dimethyl-L-phenylalaninamide (0.49 g, 0.88 mmol, Compound 7) in tetrahydrofuran (11 mL) and water (3 mL), hydrogen peroxide (30% aqueous solution, 0.76 mL, 7.9 mmol) followed by lithium hydroxide monohydrate (0.11 g, 2.6 mmol) are added. The reaction mixture is stirred for 23 hours while gradually warming to room temperature. The reaction is quenched by the addition of sodium sulfite (1.5 M aqueous solution, 10 mL, 15 mmol), which is accompanied by slight exothermicity. The quenched mixture is stirred for 1 hour at room temperature and then cooled to 0° C. in an ice-water bath. The pH of the mixture is adjusted to 4 by the addition of citric acid (1 M aqueous solution). The acidified mixture is then extracted thrice with ethyl acetate. The combined extracts are washed with saturated aqueous sodium chloride solution, dried over anhydrous sodium sulfate, decanted, and concentrated under reduced pressure to afford a white foam (0.47 g). This crude product is dissolved in acetonitrile/water (1:1) and purified by semi-preparative reverse-phase HPLC, employing a gradient elution of 5% acetonitrile/95% water to 100% acetonitrile over 1 hour. A hard, white foam (0.23 g, 66%) is obtained after collection and concentration. TOF MS (ES⁻): m/z (M−H)=396.2

### Synthesis of N-(tert-butoxycarbonyl)-β,β-dimethyl-4-(2-phenyl-1,3-dioxolan-2-yl)-L-phenylalanine (Compound 9)

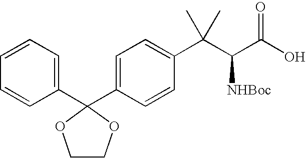

According to Scheme I, a solution of (4S)-3-{(2S)-2-azido-3-methyl-3-[4-(2-phenyl-1,3-dioxolan-2-yl)phenyl]butanoyl}-4-benzyl-1,3-oxazolidin-2-one (0.74 g, 1.4 mmol, Compound 6a) in ethyl acetate (25 mL) is degassed with a small piece of dry ice. When all effervescence had subsided, palladium on carbon (Pd/C, 10%, 20 mg) is added in a single portion, followed by di-t-butyl dicarbonate (0.61 g, 2.8 mmol). The reaction flask is evacuated under weak house vacuum and then flushed with hydrogen (balloon pressure). This process is repeated thrice. Finally, the reaction mixture is allowed to stir under a hydrogen atmosphere. After 4 hours, TLC showed a complete disappearance of starting material and the emergence of a single new spot of lower retention factor. The reaction mixture is filtered through a Diatomaceous earth pad to remove Pd/C. The filtrate is concentrated under reduced pressure to afford (1.3 g, >100%) of a clear, light blond oil.

To a 0° C. solution of this crude material (1.3 g, 1.4 mmol maximum) in tetrahydrofuran (17 mL) and water (4 mL), hydrogen peroxide (30% aqueous solution, 1.4 mL, 13 mmol) followed by lithium hydroxide monohydrate (0.18 g, 4.2 mmol) are added. The reaction mixture is stirred for 23 hours at room temperature. LC/MS analysis revealed the reaction to be incomplete. The mixture is cooled to 0° C. and an additional 4 mL of hydrogen peroxide solution and 0.18 g of lithium hydroxide monohydrate is added. Stirring is continued for 60 hours while the mixture gradually warmed to room temperature. The reaction is quenched by the addition of sodium sulfite (1.5 M aqueous solution, 25 mL, 38 mmol), which is accompanied by slight exothermicity. The quenched mixture is stirred for 1 hour at room temperature and then cooled to 0° C. in an ice-water bath. The pH of the mixture is adjusted to 4 by the addition of citric acid (1 M aqueous solution). The acidified mixture is then extracted thrice with ethyl acetate. The combined extracts are washed with a saturated aqueous sodium chloride solution, dried over anhydrous sodium sulfate, decanted, and concentrated under reduced pressure to afford a white foam (0.90 g). This crude product is dissolved in dimethylsulfoxide and purified by semi-preparative reverse-phase HPLC, employing a gradient elution of 5% acetonitrile/95% water to 100% acetonitrile over 1 hour. N-(tert-butoxycarbonyl)-β,β-dimethyl-4-(2-phenyl-1,3-dioxolan-2-yl)-L-phenylalanine is obtained as a white powder (0.37 g, 60%) after collection and concentration. TOF MS (ES⁻): (M−H)=440.1

7.2.2 Synthesis of Probe 1

Scheme II, below, diagrams the synthesis of 4-benzoyl-N,β,β-trimethyl-L-phenylalanyl-N¹-[(1S,2E)-3-carboxy-1-isopropylbut-2-enyl]-N¹,3-dimethyl-L-valinamide (Probe 1).

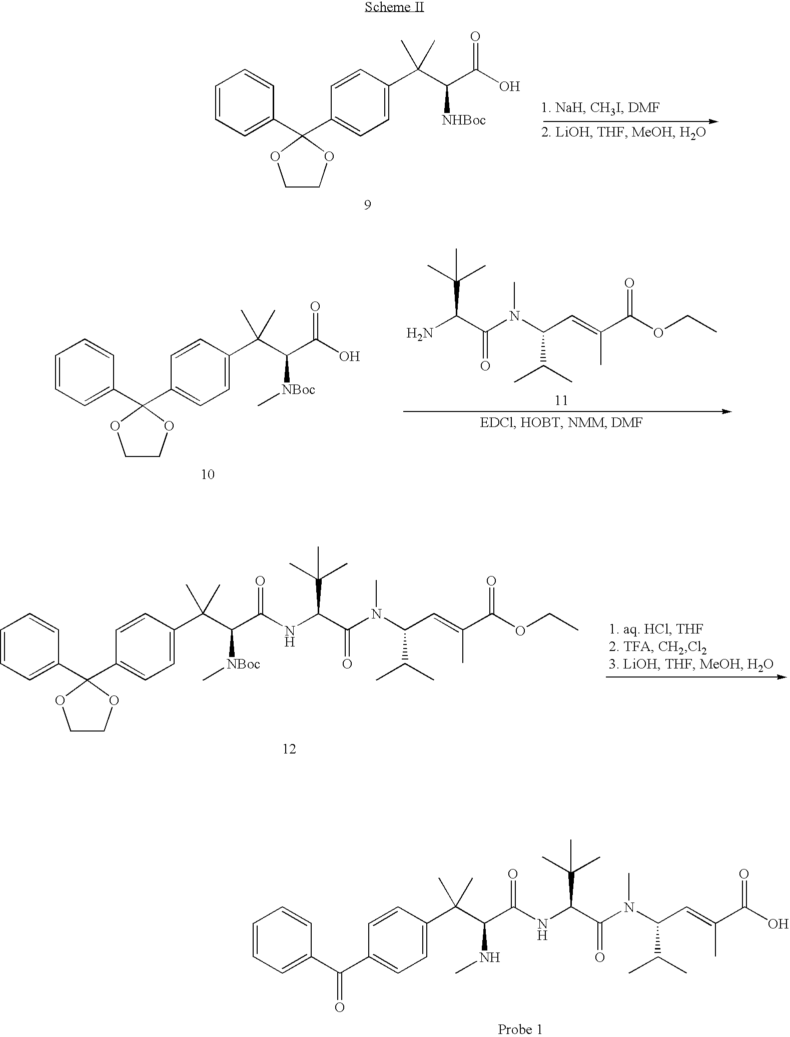

The syntheses, below, describe the preparation of the compounds shown in

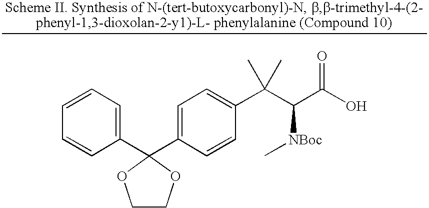

According to Scheme II, a solution of N-(tert-butoxycarbonyl)-β,β-dimethyl-4-(2-phenyl-1,3-dioxolan-2-yl)-L-phenylalanine (0.34 g, 0.77 mmol, Compound 9) in anhydrous dimethylformamide (11 mL) is cooled to 0° C. in an ice-water bath under a nitrogen atmosphere. Sodium hydride (60% dispersion in mineral oil, 0.15 g, 3.9 mmol) is added slowly. After effervescence has ceased, methyl iodide (0.49 g, 7.8 mmol) is added via syringe. The reaction mixture is then allowed to warm to room temperature gradually while stirring overnight. The mixture is then cooled to 0° C. in an ice-water bath. Glacial acetic acid (1 mL) is added to adjust the pH to 4. The reaction mixture is partitioned between ethyl acetate and water. The aqueous phase is extracted thrice with diethyl ether. The combined extracts are washed with saturated aqueous sodium hydrogen carbonate and saturated aqueous sodium chloride, dried over sodium sulfate, decanted, and concentrated under reduced pressure to a blond oil (0.49 g, >100%). MS (ES⁺): m/z (M+Na)=492.3

The crude blond oil (0.49 g, 0.77 mmol maximum) is taken up in tetrahydrofuran (2 mL), methanol (2 mL), and water (1 mL). To this solution, lithium hydroxide monohydrate (81 mg, 1.9 mmol) is added. The reaction mixture is stirred for 24 hours at room temperature and an additional quantity of lithium hydroxide monohydrate (20 mg, 0.48 mmol) is added. Stirring is resumed for 60 hours, following which the solvent is evaporated under reduced pressure to give a white solid (0.62 g), which is purified by semi-preparative reverse-phase HPLC, employing a gradient elution of 5% acetonitrile/95% water to 100% acetonitrile over 1 hour. N-(tert-butoxycarbonyl)-N,β,β-trimethyl-4-(2-phenyl-1,3-dioxolan-2-yl)-L-phenylalanine is obtained as a hard white foam (0.26 g, 74% over 2 steps) after collection and concentration. TOF MS (ES⁻): m/z (M−H)=454.1

### Synthesis of 4-benzoyl-N,β,β-trimethyl-L-phenylalanyl-N¹-[(1S,2E)-3-carboxy-1-isopropylbut-2-enyl]-N¹,3-dimethyl-L-valinamide (Probe 1)

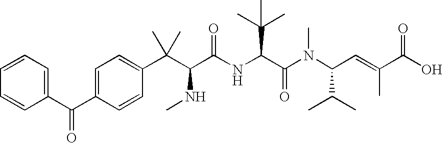

According to Scheme II, to a solution of N-(tert-butoxycarbonyl)-N,β,β-trimethyl-4-(2-phenyl-1,3-dioxolan-2-yl)-L-phenylalanine (0.23 g, 0.50 mmol, Compound 10) and ethyl (2E,4S)-2,5-dimethyl-4-[methyl(3-methyl-L-valyl)amino]hex-2-enoate (0.24 g, 0.76 mmol, Compound 11) in anhydous dimethylformamide (3 mL) under a nitrogen atmosphere, hydroxybenzotriazole (0.14 g, 1.0 mmol), 1-(3-dimethylaminopropyl)-3-ethylcarbodiimine hydrochloride (0.19 g, 1.0 mmol), and N-methylmorpholine (0.11 mL, 1.0 mmol) are added. After 24 hours, the mixture is diluted with water, and the aqueous layer is extracted with diethyl ether (3 times). The combined extracts are washed with 2% hydrochloric acid and saturated aqueous sodium chloride, dried over sodium sulfate, filtered, and concentrated in vacuo. The residue is isolated as a hard white foam (0.34 g, 92%, Compound 12). MS (ES⁺): m/z (M−Boc+H)=650.4

To a 0° C. solution of this material in tetrahydrofuran (10 mL), 10% hydrochloric acid (2.5 mL) is added. The mixture is stirred for 22 hours at room temperature then heated for 28 hours at 45-50° C. After an additional 48 hours at room temperature, the reaction mixture is carefully quenched by the addition of saturated aqueous sodium hydrogen carbonate and then extracted thrice with diethyl ether. The combined organic extracts are washed with saturated aqueous sodium hydrogen carbonate and saturated aqueous sodium chloride, dried over sodium sulfate, decanted, and concentrated under reduced pressure to afford a hard white foam (0.29 g, 91%). MS (ES⁺): m/z (M−Boc+H)=606.4

A solution of this material (0.29 g, 0.42 mmol) in dichloromethane (4 mL) is cooled to 0° C. in an ice-water bath. Trifluoroacetic acid (0.32 mL, 4.2 mmol) is added and the mixture is stirred for 30 minutes at 0° C. The cooling bath is then removed. After an additional 2 hours of stirring, an additional quantity of trifluoroacetic acid (0.20 mL) is added. After 18 hours of stirring, solvent and excess acid are removed under reduced pressure to yield 0.43 g of a hard brown foam.

This material is taken up in tetrahydrofuran (2 mL), methanol (2 mL), and water (1 mL). Lithium hydroxide monohydrate (0.16 mg, 3.8 mmol) is added and the reaction mixture is stirred overnight at room temperature. The solvent is evaporated under reduced pressure. The residue is purified by semi-preparative reverse-phase HPLC (employing a gradient elution of 5% acetonitrile/95% water/0.1% trifluoroacetic acid to 100% acetonitrile over 1 hour) to give a hard, white foam (0.20 g). Subsequent isocratic reverse phase HPLC purifications (employing 60% methanol/40% water (0.02%) trifluoroacetic acid) furnished 4-benzoyl-N,β,β-trimethyl-L-phenylalanyl-N′-[(1S,2E)-3-carboxy-1-isopropylbut-2-enyl]-N¹,3-dimethyl-L-valinamide trifluoroacetic acid as a white powder (60 mg, 19%). TOF MS (ES⁺): m/z (M+H)=578.4

7.2.3 Synthesis of Probe 2

Scheme III, below, diagrams the synthesis of N,β,β-trimethyl-L-phenylalanyl-4-benzoyl-N-[(1S,2E)-3-carboxy-1-isopropyl-2-butenyl]-N,β,β-trimethyl-L-phenylalaninamide (Probe 2).

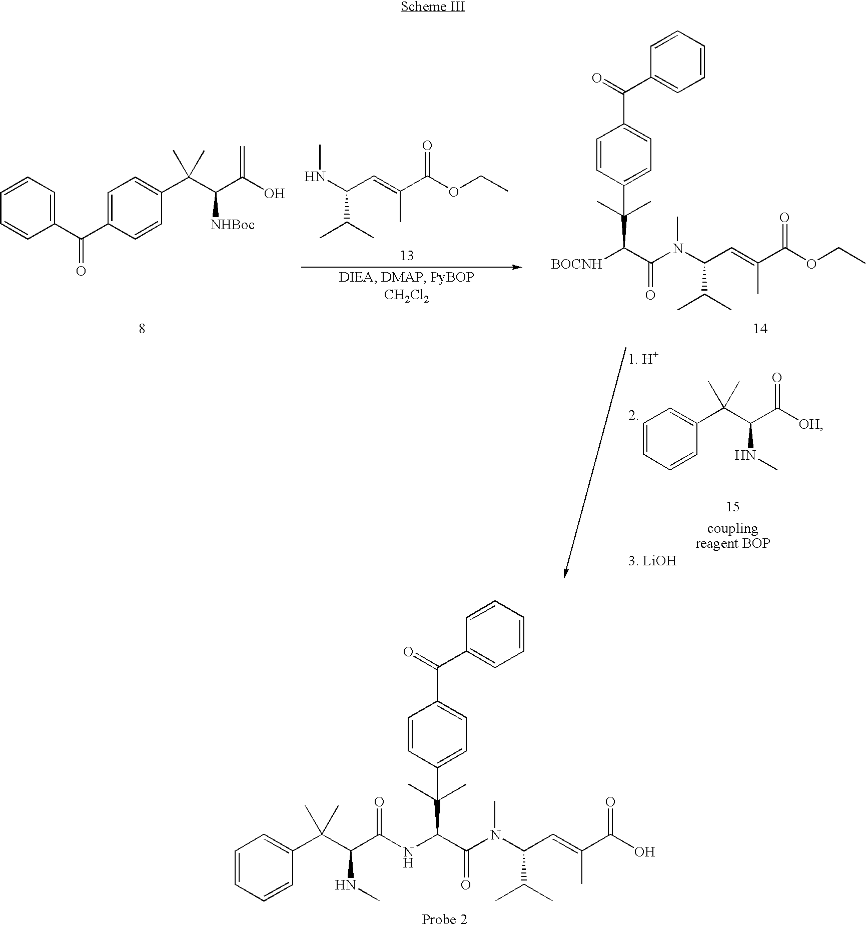

The syntheses, below, describe the preparation of the compounds shown in Scheme III.

### Synthesis of N,β,β-trimethyl-L-phenylalanyl-4-benzoyl-N-[(1S,2E)-3-carboxy-1-isopropyl-2-butenyl]-N,β,β-trimethyl-L-phenylalaninamide (Probe 2)

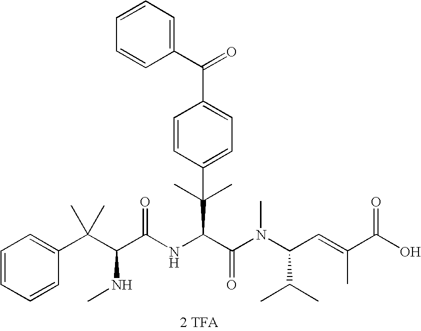

According to Scheme III, to a solution of 4-benzoyl-N-(tert-butoxycarbonyl)-β,β-dimethyl-L-phenylalanine (0.13 g, 0.33 mmol, Compound 8), benzotriazole-1-yl-oxy-tris-pyrrolidinophosphonium hexafluorophosphate (0.26 g, 0.50 mmol), and dimethylaminopyridine (DMAP, 24 mg, 0.20 mmol) in dichloromethane (4 mL, Aldrich), diisopropylethylamine (0.17 mL, 0.99 mmol) is added under a nitrogen atmosphere. To this mixture is added a solution of ethyl (E,4S)-2,5-dimethyl-4-(methylamino)-2-hexenoate (0.38 g, 1.1 mmol, Compound 13) in anhydrous dichloromethane (3 mL). The resulting reaction mixture is stirred at room temperature for 18 hours. Volatiles are evaporated under reduced pressure. The crude product (0.70 g) is purified by semi-preparative HPLC (employing a gradient elution of 5% acetonitrile/95% water to 100% acetonitrile over 1 hour). A hard, white foam (0.16 g, 84%, Compound 14) is obtained after collection and concentration. MS (ES⁺): m/z (M+Na)=601.3

This hard white foam (0.16 g, 0.28 mmol) in anhydrous dichloromethane (3 mL) is cooled to 0° C. in an ice-water bath and hydrochloric acid (4 N solution in dioxane, 1 mL, 4 mmol) is added. Stirring at 0° C. is continued for 5 minutes and then the cooling bath is removed. After 1 hour, TLC revealed a preponderance of starting material. An additional 2 mL of 4N hydrochloric acid is added. After 2 hours, the reaction mixture is still composed of mostly starting material. The reaction mixture is left in a −10° C. freezer for 72 hours, following which the TLC showed no change. An additional 2 mL of 4 N hydrochloric acid is added and the reaction mixture is allowed to stir for 8 hours at room temperature. LC/MS analysis showed the conversion to product to be nearly complete. Stirring is continued overnight and after which the ratio is unchanged. Solvent and excess acid are removed under reduced pressure. The resulting light beige foam is triturated with ether, but a gum resulted. Under vacuum, the gum afforded (0.17 g, >100%) a light beige foam, which is carried on to the next step without further purification.

To a solution of N,β,β-trimethyl-L-phenylalanine (0.17 g, 0.56 mmol, Compound 15) and the hydrochloride from the previous step (0.28 mmol max) in anhydrous dimethylformamide (4 mL) under a nitrogen atmosphere, hydroxybenzotriazole (0.076 g, 0.56 mmol, 1-(3-dimethylaminopropyl)-3-ethylcarbodiimine hydrochloride (0.11 g, 0.56 mmol) and N-methylmorpholine (0.62 μL, 0.56 mmol) are added. After 24 hours the mixture is diluted with water, and the aqueous layer is extracted with diethyl ether (3 times). The combined organic extracts are washed with saturated aqueous sodium hydrogen carbonate and saturated aqueous sodium chloride, dried over sodium sulfate, filtered and concentrated in vacuo. The crude residue is isolated as cloudy beige semi-solid (0.36 g). MS (ES⁺): m/z (M+Na)=790.5

A solution of this material (0.36 g, 0.28 mmol maximum) in dichloromethane (5 mL) is cooled to 0° C. in an ice-water bath. Trifluoroacetic acid (1.5 mL) is added and the mixture is stirred for 10 minutes at 0° C. and then the cooling bath is removed. After an additional 2 hours of stirring, thin-layer chromatography (TLC) showed complete deprotection. Solvent and excess acid are removed under reduced pressure to yield 0.52 g of a reddish oil. The oil is taken up in tetrahydrofuran (2 mL), methanol (2 mL), and water (1 mL). Lithium hydroxide monohydrate (35 mg, 0.84 mmol) is added and the reaction mixture is stirred at 45° C. for 2 hours. LC/MS analysis showed only slight hydrolysis of the ester. An additional 20 mg of lithium hydroxide monohydrate is added and the reaction mixture is heated at 55° C. overnight. The solvent is evaporated under reduced pressure. The residue is purified by semi-preparative reverse-phase HPLC (employing a gradient elution of 5% acetonitrile/95% water/0.1% trifluoroacetic acid to 100% acetonitrile over 1 hour) to give N,β,β-trimethyl-L-phenylalanyl-4-benzoyl-N-[(1S,2E)-3-carboxy-1-isopropyl-2-butenyl]-N,β,β-trimethyl-L-phenylalaninamide trifluoroacetic acid as a hard, white foam (0.12 g, 50% over 4 steps). TOF MS (ES⁺): m/z (M+H)=640.4

TABLE 1
Photoaffinity Probes for HTI-286

| | | | | tubulin |
|---|---|---|---|---|
| | | K_{D} | IC₅₀ | polymerization |
| Compound | Structure | (μM)† | (nM)† | (% inhibition)† |
| HTI-286 | 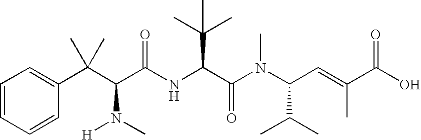 | 0.4-0.8 | 0.96 ± 0.5 (n = 79) | 87.5 ± 12.2 (n = 49) |
| Probe 1 | 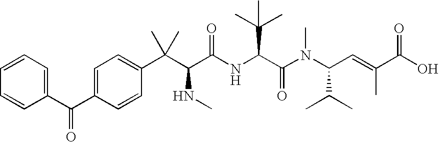 | 0.2-0.8 | 1.8 ± 0.1 (n = 2) | 88 |
| Probe 2 | 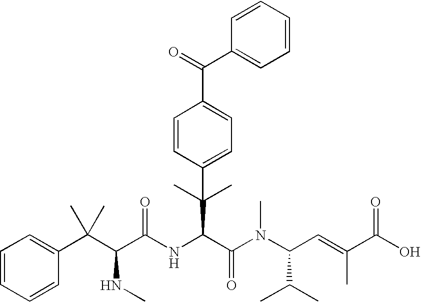 | 1.1-6 | 22.4 ± 0.7 (n = 2) | 69 |

†Data are from experiments described in Section 7.3, below.

7.2.4 Radiolabeling of Probe 1 and 2

Probes 1 and 2 were tritium labeled by direct tritium exchange labeling of the corresponding unlabeled compounds using Crabtree's catalyst, (1,5-cyclooctadiene)(pyridine)(tricyclohexylphosphine)iridium(I) hexafluorophosphate (Crabtree et al., J. Am. Chem. Soc. 1982, 104:6994-7001; Crabtree, Acc. Chem. Res. 1990, 23:95-101). This catalyst is commonly used for the introduction of tritium labels into aromatic positions, especially those positions ortho to benzophenone carbonyl groups (Heys et al., J. Chem. Soc. Chem. Commun. 1992, 9:680-681; Hesk et al., J. Label Compd. Radiopharm. 1995, 36:497-502; Shu et al., J. Organometallic Chem. 1996, 524:87-93; Chen et al., J. Label Compd. Radiopharm. 1997, 39:291-298; Shu et al., J. Label Compd. Radiopharm. 1999, 42:797-807). A solution of unlabeled Probe 1 TFA salt (1 mg, 1.6 μmol) and Crabtree's catalyst (5 mg, 6.4 μmole, 4.0 equiv; purchased from Aldrich (St. Louis, Mo.)), in methylene chloride (1.00 mL) was exposed to 3.90 Curies of pure tritium gas (0.20 atmosphere) overnight followed by work-up, removal of all volatile tritium, and HPLC purification on a Phenomenex Prodigy 5 um ODS(3) semi-preparative HPLC column (250 mL×10 mm ID) with an aqueous acetonitrile (0.02% TFA) gradient. A total of 89.9 mCi of tritiated Probe 1 was recovered with radiochemical purity >98%. The specific activity was determined to be 70.6 Ci/mmol by LC/MS, with an average of 2.45 tritium atoms per molecule. Proton (600 MHz) and tritium (640 MHz) NMR (in d₆-DMSO solvent) established that the labels were in aromatic positions as expected. Similarly, a solution of unlabeled Probe 2 bis TFA salt (1.4 mg, 1.6 μmole) and Crabtree's catalyst (6.5 mg, 8.0 μmole, 5.0 equiv) in methylene chloride (1.00 mL) was exposed to 3.79 Curies of pure tritium gas (0.21 atmosphere) overnight followed by work-up, removal of all volatile tritium, and HPLC purification as described above. A total of 93.5 mCi of tritiated Probe 2 was recovered with a radiochemical purity >98%. The specific activity of tritiated Probe 2 was 82.1 Ci/mmol as determined from LC/MS, with an average of 2.80 tritium atoms per molecule. Similar proton and tritium NMR analysis (also in d₆-DMSO solvent) confirmed the presence of the tritium atoms in aromatic positions.

7.3. Binding and Activity Studies of HTI Analog Probes

This example describes binding and activity studies of HTI analogs that are referred to here as Probes 1 and 2, and are described in the Examples at sections 7.2.1 and 7.2.2, supra. The results of these studies, including binding affinity, tubulin polymerization, and cell proliferation assays, demonstrate that the HTI analogs of this invention interact with tubulin and have other properties similar to HTI-286. The results additionally demonstrate that these analogs inhibit tubulin polymerization with a potency that is at least comperable to the potency reported for HTI-286 and for other anti-microtubule agents (Hamel, Med. Res. Rev. 1996, 16:207-231).

7.3.1 Reversible Binding Affinity

Using the fluorescence method described supra, the apparent binding constants (K_{D}) for Probes 1 and 2 were determined to be 0.5 and 3.6 μM, respectively. The K_{D} obtained for Probe 1 is approximately equivalent to the K_{D} value of 0.6 μM obtained for HTI-286. The K_{D} obtained for Probe 2 is approximately 6-fold higher than the K_{D} value obtained for HTI-286. These results suggest that Probe 1 and Probe 2 have binding affinities for tubulin that are at least comparable to the affinity of their parent compound HTI-286.

Applying the radiolabeled probe technique described supra, an apparent K_{D} value for Probe 1 of 0.35 μM was determined. This result, which is in close agreement with the value determined in the fluorescence binding assay (supra), further verifies that Probe 1 and HTI-286 have similar binding affinities.

7.3.2 Tubulin Polymerization in a Cell-Free System

As described supra, tubulin polymerization in a cell-free system was performed in the presence or absence of test compound to determine the extent of tubulin polymerization inhibition by test compounds. Consistent with reversible binding affinity results, the compounds were good inhibitors of microtubule-mediated phenomena according to the above assay. In particular, 0.3 μM HTI-286, Probe 1, and Probe 2 inhibited tubulin polymerization in a cell-free system by 88%, 88%, and 69%, respectively.

7.3.3 Cell Proliferation in Tissue Culture

According to methods supra, cell proliferation in tissue culture was determined in the presence or absence of test compound to determine the extent of cell proliferation inhibition by test compounds. Probes 1 and 2 inhibit the proliferation of KB cells in tissue culture with potency values (IC₅₀=1.8 nM and 22 nM, respectively) that are comparable to the potency of HTI-286 (IC₅₀=0.96 nM)

7.4. Photolabeling of Tubulin Using Hemiasterlin Analog Probe 1

7.4.1 Probe 1 Photolabels the α-Tubulin Subunit

The binding of Probe 1 to tubulin was evaluated by incubating tubulin derived from bovine brain (2.5 μM) or HeLa cells (10 μM) with 2.5 μM (3.6 μCi) of [³H]-Probe 1 for 30 minutes at room temperature followed by irradiation at 360 nm for two hours at 4° C. Two hours of irradiation was chosen since initial experiments indicated that photoincorporation reached maximum levels at this time.

As noted above, irradiation of the benzophenone derivative probes at this wavelength effectively activates the photoprobe with minimal destruction of the protein. At the same time, and in contrast to other photolabeling compounds (e.g. with azido-containing photoprobes), irradiation of the benzophenone derivative probes used here leads to an excited state that reverts back to the ground state in the absence of an abstractable proton. This property, combined with the short lifetime of the excited state(s) of these probes, is understood to preclude the possibility of “false labeling” (i.e. of cross-linking with a region of the protein that is not a true labeling site).

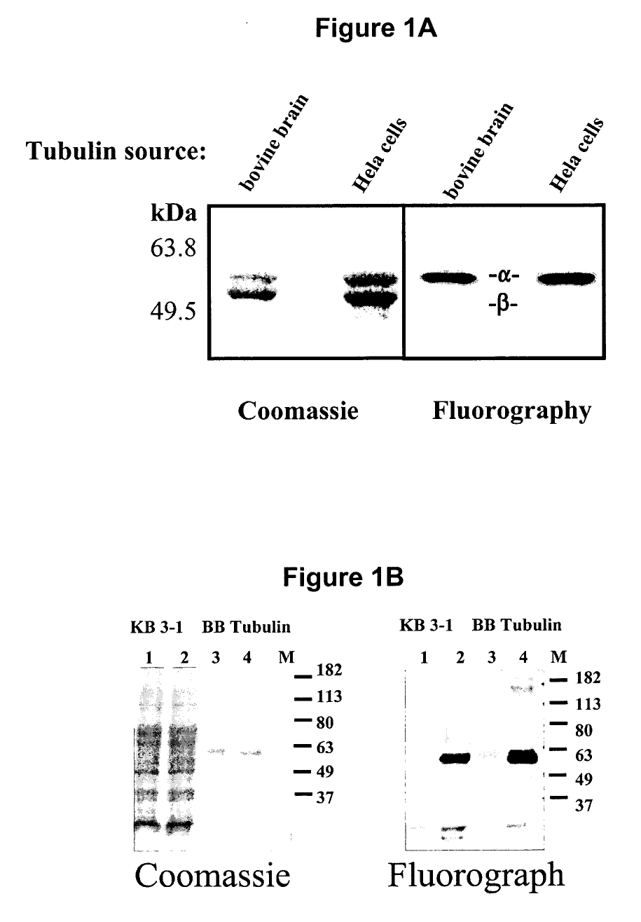

α and β-tubulin were resolved by SDS-PAGE and analyzed by Coomassie blue staining or fluorography of the gel as described above. According to FIG. 1A, the Coomassie blue staining reveals an α- and β-tubulin band for both the bovine and Hela cell tubulin used in this example. In contrast, the audioradiograph revealing radioactive, cross-linked probe yields a band for only the α-tubulin derived from human and bovine sources. Hence, Probe 1 binds exclusively to α-tubulin. Similar results are observed when the irradiation time is varied between 5 minutes and 4 hours. Any labeling observed to co-migrate with β-tubulin is detected only after the species labeling α-tubulin is grossly overexposed and can not be definitively distinguished from α-tubulin. The identification of the higher of the two molecular weight species within the gels can be confirmed to be α-tubulin using monoclonal antibodies specific for α and β-tubulin, as well as by mass spectrometric analysis of the α and β-tubulin bands.

Probe 1 also specifically labels tubulin when co-incubated with cytosolic preparations from cell lysates that contain numerous other proteins (FIG. 1B). As shown in lane 1 of the fluorograph in FIG. 1B, non-radiolabeled Probe 1 can inhibit the binding of radiolabled Probe 1 in these lysates.

In addition, Probe 1 specifically labels tubulin when incubated in the presence of whole KB-3-1 cells. The fluorograph pertaining to this experiment reveals two bands for the sample incubated only with radiolabeled Probe 1, a 50 kDa band that co-migrates with purified tubulin and a 65 kDa band. In a sample pre-incubated with non-radiolabeled HTI-286 before exposure to radiolabeled Probe 1, the fluorograph shows only the band at 65 kDa. Thus, competition with non-radiolabeled HTI-286 eliminates the specific labeling of the 50 kDa tubulin band. Bands in the fluorographic image at 65 kDa show that radiolabeled Probe 1 non-specifically labels cells, since this band remains in the fluorograph when the sample is pre-incubated with non-radiolabeled HTI-286.

7.4.2 HTI-286 Inhibits the Photolabeling of Tubulin by Probe 1 Further Confirming that Probe 1 Acts in a Similar Manner Compared to its Parent Compound

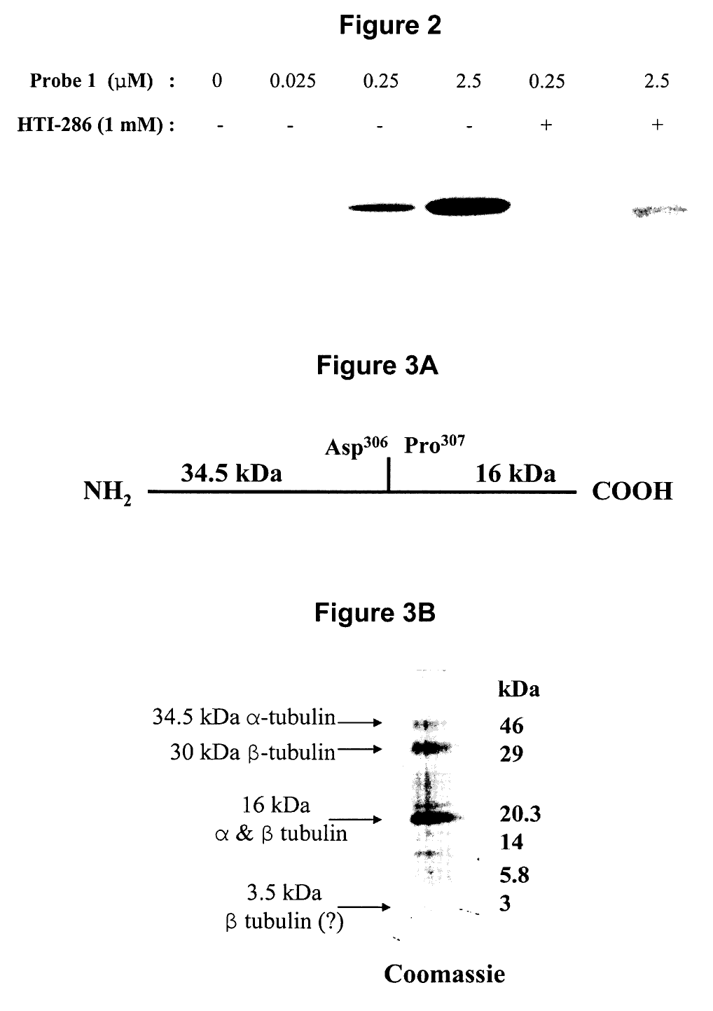

The specificity of photolabeling by Probe 1 was demonstrated by pre-incubating bovine brain tubulin (2.5 μM) with 1 mM HTI-286 for 15 minutes at 4° C., followed by addition of increasing concentrations of radiolabeled Probe 1 (0.025-2.5 μM) as described above. After incubation for 30 minutes at 37° C., samples were irradiated with 360 nm UV light for 30 minutes and separated by SDS-PAGE. Gels were exposed to film using fluorographic techniques. Under these conditions, 1 mM HTI-286 completely inhibits the incorporation of 0.25 μM radiolabeled Probe 1 into α-tubulin (FIG. 2, compare Lanes 3 and 5) and inhibits the incorporation of 2.5 μM radiolabeled Probe 1 into α-tubulin by more than 50% (FIG. 2, compare Lanes 4 and 6). α-tubulin was the only species labeled and no detection of β-tubulin labeling was found under any condition confirming that Probe 1 binds exclusively to α-tubulin.

Photolabeling specificity for Probe 1 was also confirmed by labeling tubulin in the presence of increasing concentrations of radiolabeled Probe 1 in the presence or absence of 100 μM non-radiolabeled probe 1. When a ratio of tubulin: radiolabeled probe 1 of 2.5:0.25 (10:1) was used, tubulin labeling was inhibited 93% by non-radiolabeled probe 1. When the ratio was changed to 2.5:2.5 (1:1), conditions used mostly for peptide mapping studies, tubulin labeling was inhibited 82%.

7.4.3 Temperature Effects of Probe 1 Photolabeling of Tubulin

Because the polymerization state of tubulin can be affected by temperature (for example, see Johnson and Borisy, J. Mol. Biol. 1979, 133:199-216), experiments were also performed to determine whether the binding of photolabeling probes, such as Probe 1, is also affected by temperature. In particular, photolabeling experiments were performed as described in section 7.1.5 above, at both room temperature and at 37° C. These experiments were conducted by pre-incubating tubulin in either the presence or absence of various competitors (including non-radiolabeled Probe 1, dolastatin-10, HTI-286, vinblastine, paclitaxel, or colchicine) before incubation with radiolabeled Probe 1. Results from these experiments are summarized in Table 2, infra, at the end of this Section.

Initial incubations were done at RT using 2.5 μM tubulin and 0.25 μM radiolabeled Probe 1. Under these conditions, a 400-fold molar excess (100 μM) of either non-radiolabeled Probe 1 or dolastatin-10 reduces labeling by approximately 87% whereas either HTI-286 or vinblastine reduces labeling by approximately 72%. In contrast, pre-incubation with either paclitaxel or colchicine has almost no effect on the observed photolabeling of tubulin (Table 2).

When incubations are performed at 37° C., non-radiolabeled Probe 1 and dolastatin-10 are still the best inhibitors and both reduce labeling by about 75%. HTI-286 and vinblastine both reduce labeling by about 50%. However, pacliatxel and colchicine actually enhance labeling by 73 and 119%, respectively, at this temperature—in contrast with the effect of those compounds at room temperature. The unusual effect of colchicine on Probe 1 binding to tubulin was further investigated by pre-incubating tubulin with 0, 200, 400, 1000, and 4000-fold molar excess of colchicine prior to labeling tubulin with Probe 1 at 37° C. Under these conditions, colchicine maximally enhances binding of the probe when used at a 400-fold molar excess (100 μM), has less enhancing effects at a 1000-fold molar excess (250 μM), and has no effect when used at a 4000-fold excess (1 mM).

TABLE 2
Competition of Probe 1 Binding to Tubulin

Inhibition (%)

| Competitor | Room Temp. | 37° C. |
|---|---|---|
| Control (no competitor) | 100 | 100 |
| Probe 1 | 12 ± 7.8 | 24^{†} |
| Dolastatin-10 | 13 ± 3 | 26^{†} |
| HTI-286 | 27 ± 4 | 47^{†} |
| Vinblastine | 28 ± 1 | 52 ± 0.5 |
| Paclitaxel | 83 ± 41 | 173 ± 7 |
| Colchicine | 118 ± 17 | 219 ± 14 |

^{†}Results from a single experiment.

7.4.4 Inhibition of Probe 1 Photolabeling by Various Compounds

The potency of the inhibitory effect of dolastatin-10, HTI-286, and vinblastine on photolabeling was further investigated by determining IC₅₀ values for these candidate inhibitors (i.e. the concentration of these compounds that inhibit Probe 1 photolabeling of tubulin by 50%). The same reaction conditions, using incubation at room temperature, was done as described in Section 7.4.3, above. Under these conditions, Probe 1 and dolastatin-10 are equally potent inhibitors of tubulin photolabeling (IC₅₀=5 μM). HTI-286 is slightly less potent (IC₅₀=7 μM), while vinblastine is the least inhibitory of these compounds (IC₅₀=22 μM).

7.4.5 Effect of GTP on Probe 1 Photolabeling of Tubulin

It has been previously reported that hemiasterlin and dolastatin-10 interact with tubulin to inhibit GTP exchange, but do not inhibit the ability of GTP to bind to the protein (see, for example Bai et al., Biochemistry 1999, 38:14302-14310; Bai et al., J. Biol. Chem. 1990, 265:17141-17149). Therefore experiments were also performed to determine whether GTP inhibits binding of Probe 1 to tubulin. In particular, tubulin samples were pre-incubated with a range of different GTP concentrations (between 0 and 100 μM) before photolabeling with Probe 1 as described above. However, GTP did not significantly inhibit incorporation of this probe, further indicating that Probe 1 of this invention interacts with tubulin via the same mechanism as its parent hemiasterlin derivative.

7.5. Identification of the Hemiasterlin Binding Site in Tubulin Using Probe 1

The experiments described, supra, demonstrate that the HTI analogs of this invention (e.g. Probes 1 and 2) bind tubulin with properties that are similar to hemiasterlin. Accordingly, this section describes experiments that identify the binding site of hemiasterlin and/or other hemiasterlin analogs (for example, HTI-286) to tubulin. In particular, experiments are described using radiolabled and/or photo-probe of the invention, Probe 1, to identify the tubulin binding site. The cross-linking efficiency for Probe 1 was approximately 1.5% under optimal conditions and found to be sufficient for peptide mapping studies.

7.5.1 Formic Acid Digestion after Probe 1 Photoaffinity Labeling

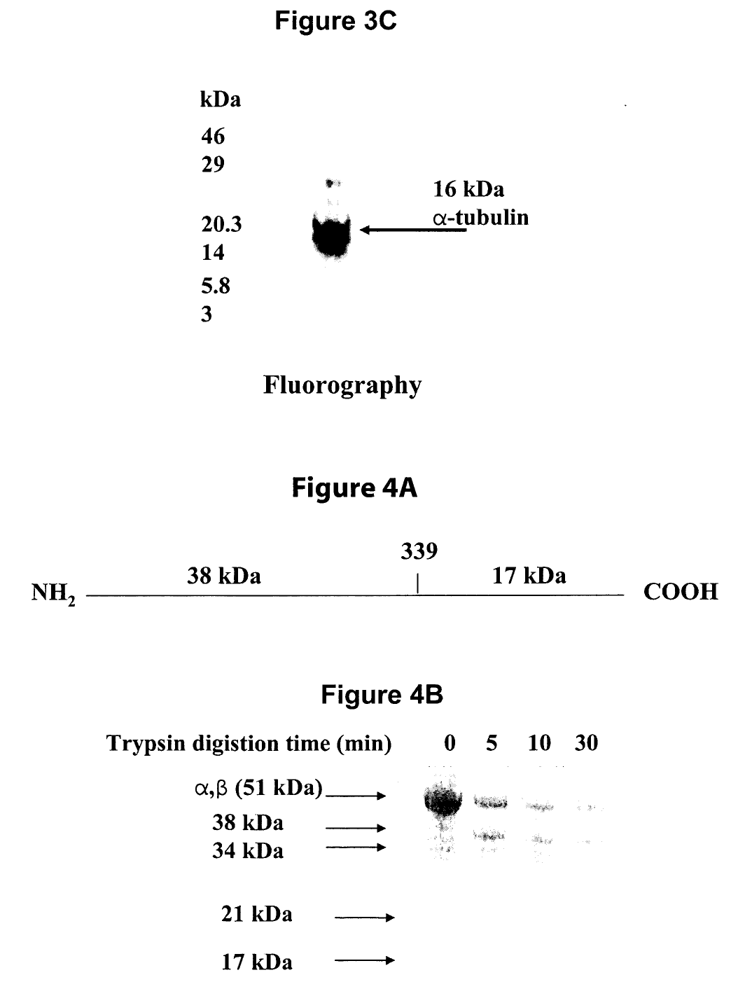

To ascertain an initial labeling region for Probe 1 in α-tubulin, formic acid digestions were performed. Formic acid is known to preferentially cleave Asp-Pro bonds (Sonderegger et al., Anal. Biochem. 1983, 122:298-301). α-tubulin is extraordinarily conserved across vertebrate species, including those derived from porcine, rat, murine, and human origin, and contains only one such linkage at Asp³⁰⁶-Pro³⁰⁷ (FIG. 3A). Therefore, complete formic acid digestion of α-tubulin derived from bovine tubulin would be expected to produced two distinct peptide fragments consisting of amino acids 1-306 (˜34.5 kDa) and 307-451 (˜16 kDa) as obtained from the digestion of other vertebrate species (e.g. the species whose tubulin amino acid sequences are depicted in FIGS. 12A-12B). These fragments have been previously identified by immunological methods as the N- and C-terminus portions of tubulin, respectively (Chau et al., Biochemistry 1998, 37:17692-17703). In contrast, formic acid digestion of β-tubulin is known to produce three fragments consisting of amino acids 1-31 (˜3.5 kDa), 32-304 (˜31 kDa) and 305-445 (˜16 kDa) (Hall et al. Mol. Cell Biol. 1983, 3:854-862). Upon formic acid in-gel digestion of photolabeled tubulin as described in Section 7.1.6, three main protein bands were observed (FIG. 3B): the upper approximately 34.5 kDa band corresponding to a fragment consistent with α-tubulin digestion, the middle band at approximately 30 kDa originating from the β-tubulin digestion, and a strongly-staining Coomassie band at approximately 16 kDa corresponding to two 16 kDa fragments originating from the α and β-tubulin digestions. Radiolabel derived from Probe 1 was found solely in the 16 kDa peptide fragment (FIG. 3C). This results is in contrast to a previously described paclitaxel analog that labeled the 31 kDa peptide fragment derived from β-tubulin (Rao et al, J. Bio. Chem. 1995, 270:20235-20238). Mass spectrometric methods confirmed that the labeled 16-kDa formic acid fragment was from α-tubulin.

7.5.2 Trypsin Digestion of Native Tubulin after Probe 1 Photoaffinity Labeling

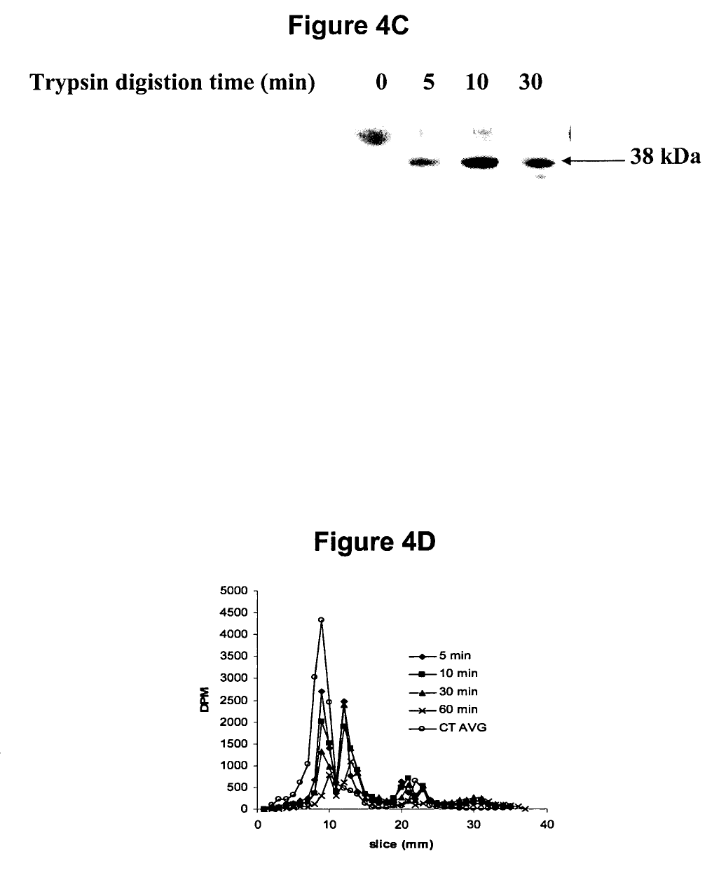

To define more narrowly the binding region of hemiasterlin and its analogs, trypsin digestion of native tubulin was performed after Probe I photoaffinity labeling as described in Section 7.1.7. Under these conditions and due to the high sequence conservation across vertebrate species, it can be predicted that trypsin preferentially cleaves α-tubulin after amino acid residue 339, producing two fragments that migrate at approximately 38 and 17 kDa (FIG. 4A) (Sacket and Wolff, J. Biol. Chem. 1986, 261:9070-9076; Serrano et al., J. Biol. Chem. 1984, 259:6607-6611; Mandelkow et al., J. Mol. Biol. 1985, 185:311-327). The latter fragment has a predicted molecular weight of 14 kDa, but migrates aberrantly in gels (Chau et al., Biochemistry 1998, 37:17692-17730). In the experiments, photolabeled tubulin was digested for 5, 10, and 30 minutes with trypsin and the resultant fragments were resolved in gels under conditions that allow α- and β-tubulin to co-migrate. Both expected species were observed by analyzing the digest using Coomassie blue staining (FIG. 4B). The origin of the 34- and 21-kDa Coomassie blue bands believed to be derived from β-tubulin (Sacket and Wolff, J. Biol. Chem. 1986, 261:9070-9076). In contrast to the Commassie stained bands, the major radiolabeled species are detected at approximately 51 kDa and approximately 38 kDa and co-migrate with the Coomassie-stained species (FIG. 4C). There is a precursor-product relationship between the undigested tubulin (51 kDa) and the 38 kDa-species, such that the ratio between the 51 and 38 kDa species decreases as the incubation time with trypsin increases. Both the gel image showing radiolabeled fragments and the radioactivity profile for the gel demonstrate this relationship (FIGS. 4C and 4D, respectively). The origin of a 19 kDa radiolabeled species, which was detected in the autoradiogram of the gel (FIG. 4C) and slices of gel (FIG. 4D), is unknown. However, it was present in the undigested sample and did not increase in intensity as the time of trypsin digestion was increased from 5-30 minutes. This may be an inherent fragment in the tubulin preparation. A minor radiolabeled species that migrated at approximately 34 kDa was observed after 10 and 30 minute digestion with trypsin. It is unlikely that this corresponds to the 34 kDa derived from β-tubulin, since the 34 kDa Coomassie-stained band appeared within 5 minutes after trypsin digest, but it was not radiolabeled. Rather, it is more likely that the 34 kDa radiolabeled fragment is a partial digest of the 38 kDa fragment derived from α-tubulin. Consistent with this hypothesis, the intensity of the 34 kDa radiolabeled band increased as the 38 kDa band intensity decreased. Taken together with the formic acid digestion results, these data demonstrate that Probe 1 labels between residues 307-339 of intact α-tubulin.

7.5.3 CNBr, Trypsin, and Lys C Cleavage with Mass Spectrometric Confirmation after Photoaffinity Labeling of Tubulin by Probe 1

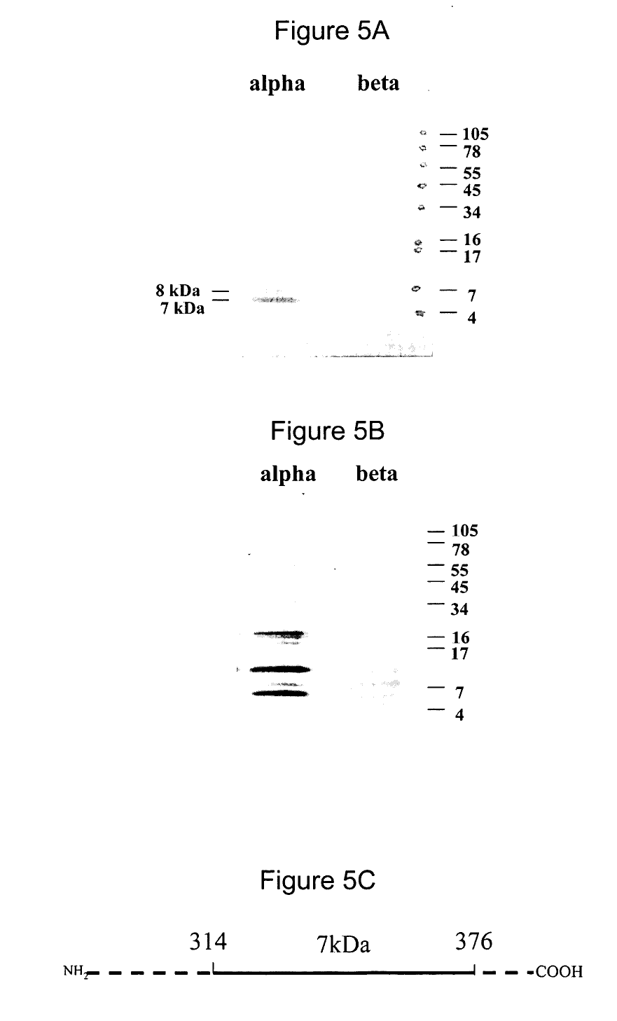

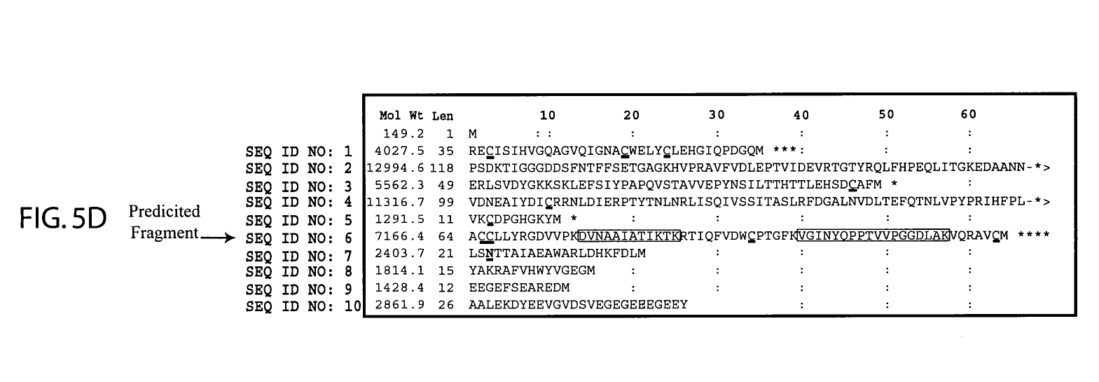

To resolve further the Probe 1 photoaffinity labeling domain, digestion of Probe 1-labeled tubulin was done with CNBr. CNBr cleaves peptide bonds on the carboxy terminus side of methionine residues and, in these studies, was used to hydrolyze peptide bonds in polyacrylamide gels (Loeb et al., Anal. Biochem. 1989, 176:365-367). Based on the sequence conservation of α-tubulin across a variety of species, it was predicted that α-tubulin would be digested into 11 fragments by CNBr (FIGS. 5C and 5D). One of these fragments, which is conserved across rat, mouse, and pig α-tubulin, has a predicted molecular weight of 7 kDa that spanned residues 314 to 376. To determine if the labeling site resided in the 7 kDa CNBr fragment as predicted from the labeling experiments above, α and β-tubulin were separated in gels and digested with CNBr according to the methods described in Section 7.1.8. Each digestion was separated on a gel and observed either by using fluorography or after silver staining of the gel (FIGS. 5A and 5B, respectively). The numerous silver-stained fragments observed in FIG. 5B indicate that the CNBr digestion of α and β-tubulin occurred. Upon fluorography of the gel, a major and minor radiolabeled species were observed at 7 and 8 kDa, respectively (FIG. 5A). Mass spectrometric results confirm that the 7 kDa fragment was correctly identified compared to the predicted results. The origin of the 8 kDa species is unknown, but likely to be a partial digest composed of the 1291 and 7166 dalton CNBr fragments (FIG. 5D). These cleavage studies further defined the binding site of Probe 1 to reside between residues 314 and 339.

7.5.4 Effects of the Labeling of α-Tubulin by Probe 1 on Subtilisin Digestion.

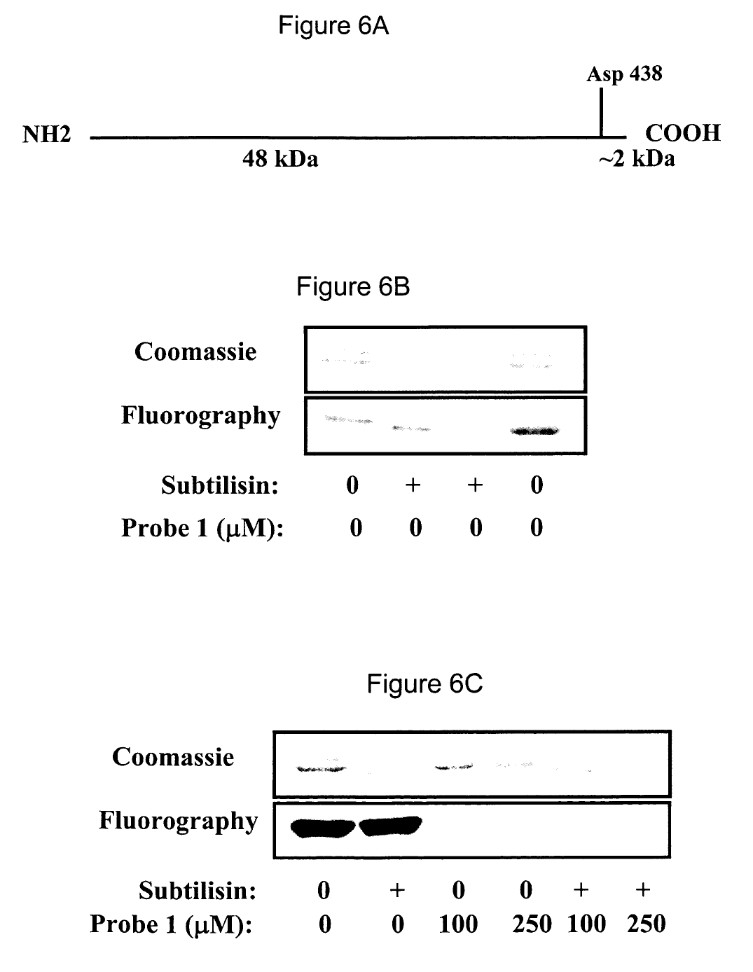

To ensure that a major labeling site was not overlooked, studies were done with subtilisin digestion before or after Probe 1 photolabeling of tubulin as described in Section 7.1.10. Knowing that c-tubulin is conserved across many species, it can be inferred that susubtilisin cleaves bovine α-tubulin between Asp-438 and Ser-439 and β-tubulin between Gln-433 and Gly-434 (Redeker et al. FEBS Lett. 1992, 313:185-192) (FIG. 6A). The cleaved protein is referred to as tubulin-S (Serrano et al., Proc. Natl. Acad. Sci. USA 1984, 81:5989-5993). Consistent with these predictions, it is observed that subtilisin digestion increases the mobility of α- and β-tubulin approximately 2 kDa as detected by Commassie staining of the material resolved in gels (FIG. 6B, compare Lanes 1 and 2, upper panel). If enzyme digestion is done before labeling, a radiolabeled band that co-migrates with the position of α-tubulin-S is detected (FIG. 6B, Lane 2). Since the intensity of radiolabeled α-tubulin-S does not decrease remarkably compared with non-digested material, especially when considering the relative amounts of Commassie-staining material that are detected, this suggests that a major photolabeling domain is unlikely to reside in the C-terminus of α-tubulin. This conclusion is consistent with the findings stated above.

To eliminate further the possibility of a C-terminus binding domain for Probe 1, the C-terminus of α-tubulin was removed by subtilisin digestion after photolabeling (FIG. 6B, Lane 3). Contrary to the expected result, α-tubulin-S is not labeled with probe 1, despite the fact that digestion did occur according to Coomassie staining. Some radiolabeled tubulin remains at the position of α-tubulin. Since vinblastine (but not maytansine) has been shown to inhibit the digestion of α-tubulin but not β-tubulin by subtilisin, and this effect may be mediated by alteration of the state of tubulin rather than blockade of the cleavage site by vinblastine (Rai and Wolff, Proc. Natl. Acad. Sci. USA 1998, 95:4253-4257), it is hypothesized that hemiasterlins perform in a similar fashion.

Upon closer inspection, it was found that 0.25 μM radiolabeled [³H]-Probe 1 partially inhibits the digestion of α-tubulin (FIG. 6C, Lane 2, Coomassie stained gel) and 100 or 250 μM non-radiolabeled Probe 1 completely blocks the ability of α-tubulin to be digested with subtilisin, while β-tubulin is completely digested (FIG. 6C, Lanes 3-6). These results suggest that the C-terminus of α-tubulin and the hemiasterlin photolabeling site interact such that the C-terminus cannot be digested with subtilisin if tubulin is pre-incubated with Probe 1. However as observed above, the C-terminus of α-tubulin does not contain a major labeling site.

7.6. Photolabeling of Tubulin Using Hemiasterlin Analog Probe 2

7.6.1 Probe 2 Photolabels the α-Tubulin Subunit

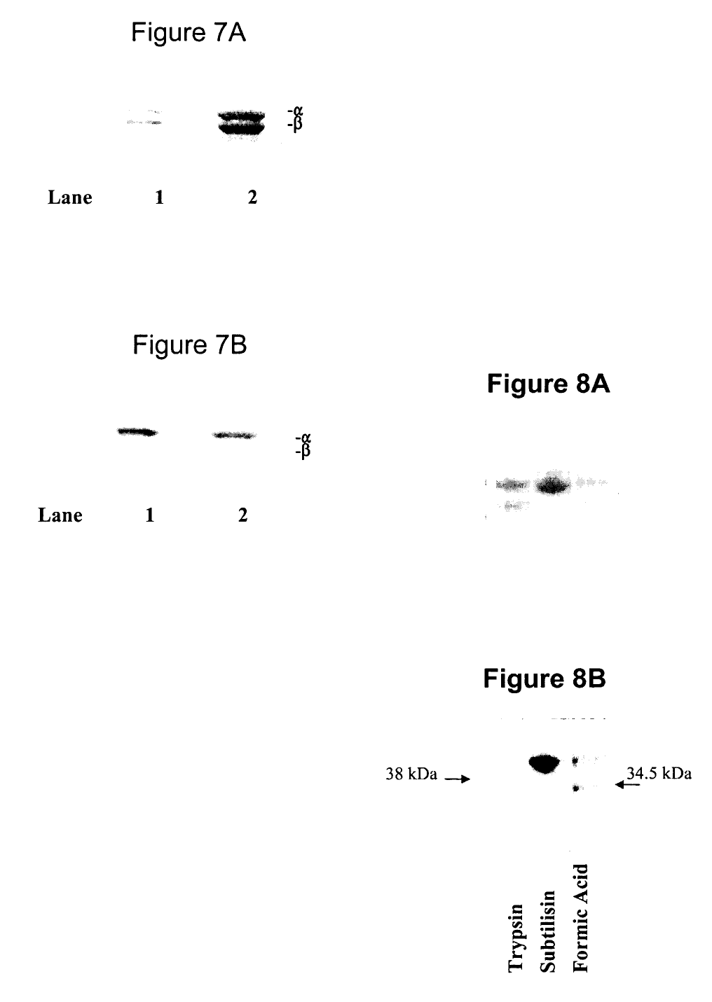

Probe 2 was observed to photolabel tubulin using the methods described in Section 7.1.5. Similar to Probe 1, Probe 2 exclusively labels α-tubulin in purified tubulin prepartions from bovine brain and HeLa cells (FIG. 7). In contrast to the Commassie blue stained gel (FIG. 7A) that displays both α and β-tubulin, the autoradiograph shows that only α-tubulin contains radiolabeled photoprobe (FIG. 7B).

7.6.2 Competition Studies Using Various Anti-Cancer Drugs as Inhibitors of Probe 2 Photolabeling

Competition binding experiments were performed as described in Section 7.1.5. Briefly, 100 μM competitor drug was pre-incubated at room temperature for 15 minutes with 2.5 μM tubulin before incubating with 0.25 μM [³H]-Probe 2 at room temperature for 30 minutes. Samples were then irradiated for 2 hours at 4° C. and evaluated by SDS-PAGE and fluorography. Non-radioactive Probe 2, dolastatin-10, HTI-286 and vinblastine can inhibit Probe 2 labeling of tubulin by 12%, 27%, 45%, and 64%, respectively. As seen in the case of Probe 1, paclitaxel and colchicine enhanced photolabeling to 113% and 107% of the control value, respectively. However, this effect was lost at higher concentrations of paclitaxel and colchicine. The ability for certain competitors to reduce or increase Probe 2 labeling demonstrates that Probe 2 specifically labels tubulin.

7.7. Identification of the Hemiasterlin Binding Site in Tubulin Using Probe 2

The experiments described, supra, demonstrate that the HTI analogs of this invention (e.g. Probes 1 and 2) bind tubulin with properties that are similar to hemiasterlin. Accordingly, this section describes experiments that identify the binding site of hemiasterlin and/or other hemiasterlin analogs (for example, HTI-286) to tubulin. In particular, experiments are described using the radiolabled photo-probe of the invention, Probe 2, to identify the tubulin binding site. These studies can be paired with Probe 1 mapping studies described in Section 7.5 to define further the binding site of hemiasterlin and its analogs.

7.7.1 Trypsin, Formic Acid and Lys C Digestion after Probe 2 Photoaffinity Labeling

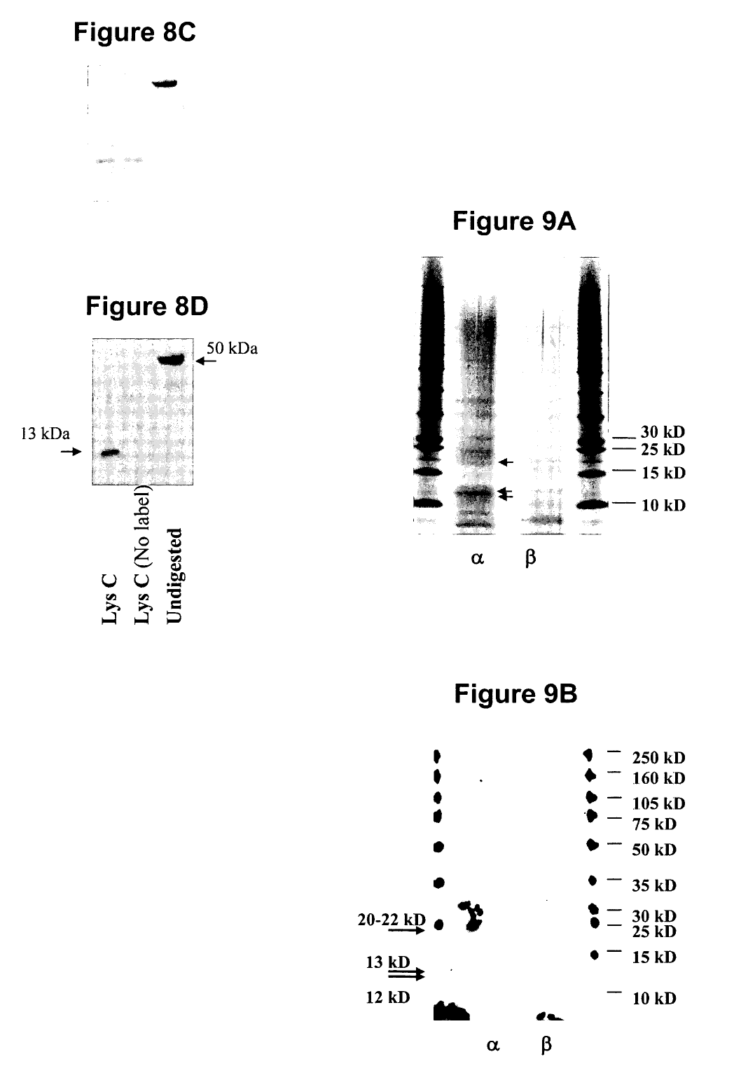

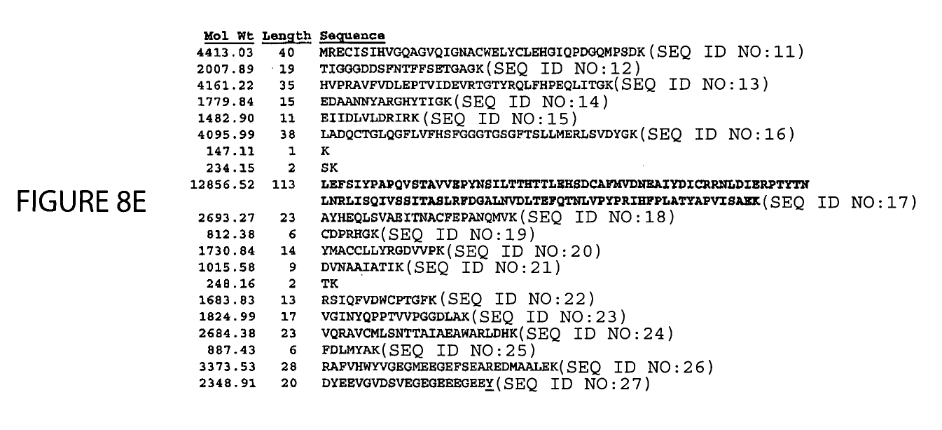

To determine the Probe 2 labeling site on α-tubulin, trypsin, formic acid, and Lys C digestions were performed on photolabeled bovine brain tubulin as described in Sections 7.1.7 and 7.1.11, respectively. As described in Section 7.5.2 and FIG. 4A, trypsin cleaves α- and β-tubulin partially under native conditions. The first fragments obtained are 38 kDa and 17 kDa for α-tubulin and 34 kDa and 21 kDa for β-tubulin. Probe 2 was found to be bound to the 38 kDa N-terminal fragment from α-tubulin spanning residue 1-338 (FIGS. 8A and 8B).

Formic acid cleavage was carried out in the presence of SDS as a denaturant such that all formic acid sites for α- and β-tubulin are accessible and digestion is complete. As described in Section 7.5.1, formic acid digestion results in the cleavage of α-tubulin into two fragments 34.5 kDa (residues 1-306) and 16 kDa (307-451) (FIG. 3A). In contrast, formic acid digestion of β-tubulin is known to produce three fragments consisting of amino acids 1-31 (3.5 kDa), 32-304 (31 kDa) and 305-445 (16 kDa). The Probe 2 radiolabel was found to label the 34.5 kDa fragment that corresponds to α-tubulin residues 1-306 (FIGS. 8A and 8B).

In a separate digestion experiment, Lys C was used to digest both α- and β-tubulin into several fragments. As predicted from the sequence homology with other vertebrate species, the largest bovine brain α-tubulin fragment was 12.8 kDa (FIG. 8E). Probe 2 was found to label this 12.8 kDa fragment corresponding to residues 167-280 (FIGS. 8C and 8D). Combining the results for the trypsin, formic acid, and Lys C digestions, Probe 2 photolabels α-tubulin between residues 167-280.

7.7.2 Subtilisin Digestion before Probe 2 Photoaffinity Labeling

To define further the photolabeling and binding site of Probe 2, subtilisin digestions were performed before Probe 2 photoaffinity labeling as discussed in Section 7.1.12. Subtilisin cleaves a 2-4 kDa C-terminal fragment from both α- and β-tubulin as described in Section 7.5.4 and FIG. 6A. After cleavage of native tubulin with subtilisin and [³H]-Probe 2 photolabeling, most of the label was present in the 48 kDa tubulin fragment (FIGS. 8A and 8B). Thus, the 48 kDa fragment contains the binding region for probe 2. This result further verifies that the binding and labeling site for Probe 2 defined by the trypsin, formic acid, and Lys C digestions is valid.

7.7.3 CNBr Cleavage after Photoaffinity Labeling of Tubulin by Probe 2

To determine more narrowly the binding site region for Probe 2, CNBr digestions according to the methods described in Section 7.1.8 were performed. As discussed in Section 7.5.3 and FIG. 5, CNBr digestion of α- and β-tubulin also gives rise to several fragments of approximately equal molecular weight. To minimize the number of probable fragments and their identity, photolabeled tubulin was separated into α- and β-tubulin subunits by SDS-PAGE, and the bands corresponding to the two proteins were digested separately with CNBr. Digested α and β-tubulin were run on gels separately and either silver stained (FIG. 9A) or subjected to fluorographic methods (FIG. 9B) as described in Section 7.1.8. The radiolabeled band with the highest intensity corresponds to a 12 kDa fragment (residues 204-302). Other bands that contain radiolabel are located at 13 kD and 20 kDa (FIG. 9B) and possibly correspond to partial digests with CNBr at residues 204-313 and 204-377 respectively. Taken together with other digests described in this section, these results suggest a binding site for Probe 2 on α-tubulin within residues 204-280.

7.8. Subtilisin Cleavage Studies for Tubulin in the Presence of Various Anti-Microtubule Agents

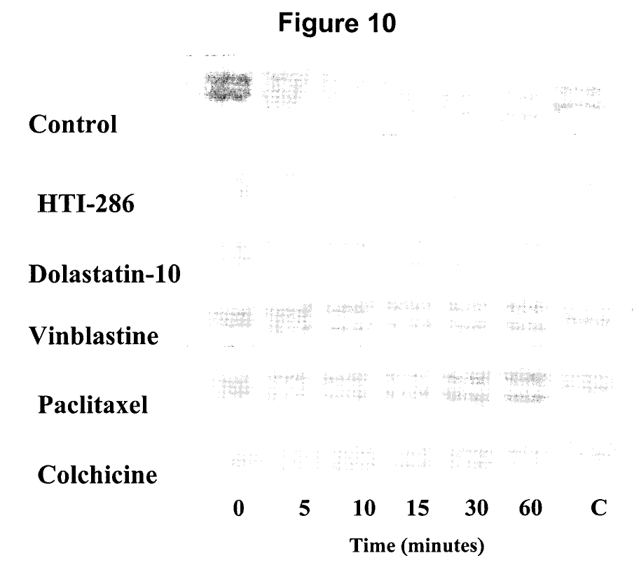

Vinblastine protects against subtilisin cleavage of α-tubulin as described in Section 7.5.4 (Rai & Wolff, Proc. Natl. Acad. Sci. U.S.A. 1998, 95:4253-4257). The hemiasterlin analog, HTI-286, was compared with other microtubule binding agents to determine if HTI-286 also protects α-tubulin against subtilisin cleavage and if this protection occurs through the same proposed mechanism as other binding agents. Methods for this experiment are described in Section 7.1.13. Partial cleavage of α- and β-tubulin by subtilisin gives rise to a 2-4 kDa shift of the two subunits as seen in SDS-PAGE gels (FIG. 10, Control). If the amount of subtilisin and time of cleavage is controlled, the β-tubulin is cleaved more rapidly than α-tubulin. When tubulin was incubated with HTI-286, dolastatin-10, vinblastine and paclitaxel, subtilisin was unable to cleave α-tubulin even after 60 minutes (FIG. 10) as seen by the lack of shifting of the α-tubulin as compared to the Control lane. Colchicine was the only drug that did not protect α-tubulin from cleavage by subtilisin.

The protection of α-tubulin by vinblastine has been attributed to the ability of drugs to induce the formation of microtubules (in case of paclitaxel) or tubulin polymers/aggregates (in the case for vinblastine and dolastatin-10). See, Rai & Wolff, Proc. Natl. Acad. Sci. U.S.A. 1998, 95:4253-4257. These aggregates can be recovered by centrifugation at high speeds as described in Section 7.1.14. When drug treated tubulin was subjected to centrifugation at 10,000 g and 100,000 g a significant amount of protein was recovered in the pellet fractions of tubulin treated with dolastatin-10, vinblastine and paclitaxel, but no significant protein was recovered in HTI-286 and colchicine fractions. Absence of pellet fractions suggests that HTI-286 does not cause aggregation of tubulin, the aggregates are not large enough, or the aggregates are unstable. Nevertheless, despite the lack of stable aggregates, HTI-286 protects α-tubulin from subtilisin cleavage. This result suggests that the mechanism of protection of α-tubulin from subtilisin cleavage by HTI-286 binding is distinct from vinblastine and dolastatin-10.

7.9. Mapping the Photoaffinity Labeling Sites for Probe 1 and Probe 2 to the Electron Micrographic Crystal Structure of Tubulin (3.5 Å)

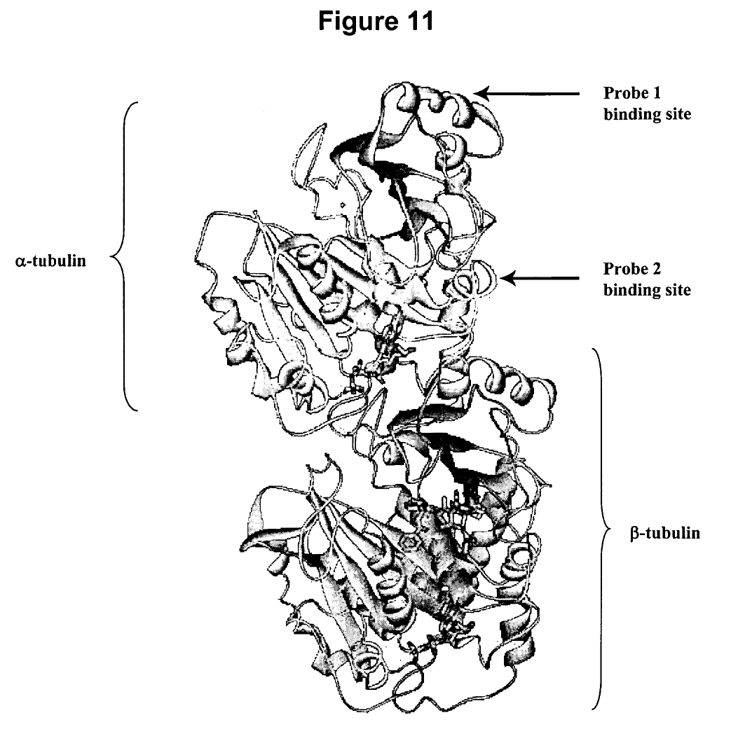

Molecular modeling studies were performed using the Weblab Viewer Lite software. The structure of tubulin was taken from Lowe et al. (J. Mol. Biol. 2001, 313:1045-1057). The major photoaffinity binding site for Probe 1 is located within residues 314-338 that, based on the electron micrographic crystal structure (3.5 Å) of zinc-induced tubulin sheets (Lowe et al., J. Mol. Biol. 2001, 313:1045-1057), corresponds to the S8 (residues 311-321) to H10 (residues 324-336) regions of α-tubulin (FIG. 11, green label). The region is perfectly conserved within porcine, rat and murine species (Stanchi et al., Biochem. Biophys. Res. Commun. 2000, 270:1111-1118, See, also, FIGS. 12A-12B) with the exception of human α-tubulin that contains a single amino acid substitution (Met¹⁷). This region is approximately 80% divergent from the corresponding region in β-tubulin within these species. The H10 domain is involved in establishing both longitudinal and lateral protofilament contacts of tubulin subunits (Nogales et al., Cell 1999, 96:79-88). Further resolution of the exact binding site within the S8-H10 region are required to refine the model, but due to the paucity of labeling we have not been able to resolve the site further.

The Probe 2 photoaffinity labeling site exists between residues 204-280 (FIG. 11, yellow label). The binding site defined by Probe 1 and 2 is a novel site for interaction of anti-mitotic drugs with tubulin. In contrast, the tubulin polymerizer paclitaxel binds in β-tubulin and the tubulin depolymerizing vinca-peptides bind the “vinca binding domain” on β-tubulin. Colchicine binds α- and β-tubulin in the intradimer interface.

### 8. REFERENCES CITED

Numerous references, including patents, patent applications and various publications, are cited and discussed in the description of this invention. The citation and/or discussion of such references is provided merely to clarify the description of the present invention and is not an admission that any such reference is “prior art” to the invention described here. All references cited and/or discussed in this specification (including references, e.g., to biological sequences or structures in the GenBank, PDB or other public databases) are incorporated herein by reference in their entirety and to the same extent as if each reference was individually incorporated by reference.

What is claimed is:

1. A compound represented by the formula: 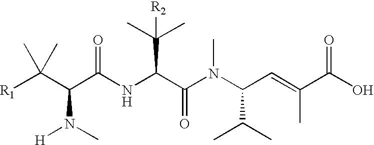
or a pharmaceutically acceptable salt thereof, in which:
(a) R₁ is a photoreactive moiety or an aryl moiety;
(b) R₂ is a photoreactive moiety, an alkyl moiety or H; and
(c) at least one of R₁ and R₂ is a photoreactive group.

2. The compound according to claim 1 in which the photoreactive group of R₁ or R₂ is a benzophenone moiety.

3. The compound according to claim 1 in which the photoreactive group of R₁ or R₂ is an azide moiety.

4. A compound according to claim 1 in which R₁ is a benzophenone moiety, and R₂ is an alkyl moiety or H.

5. A compound according to claim 1 in which R₁ is an aryl moiety and R₂ is a photoreactive group.

6. A compound according to claim 1 and having the formula: 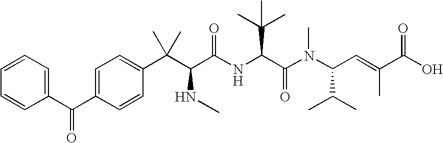
or a pharmaceutically acceptable salt thereof.

7. A compound according to claim 1 and having the formula 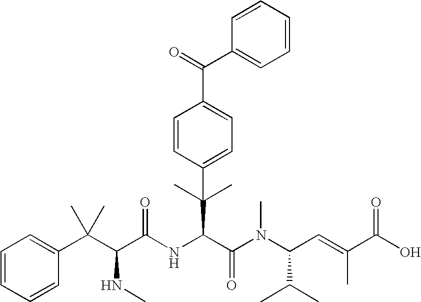
or a pharmaceutically acceptable salt thereof.

8. A compound according to claim 1 which is 4-benzoyl-N,β,β-trimethyl-L-phenylalanyl-N1-[(1S,2E)-3-carboxy-1-isopropylbut-2-enyl]-N1,3-dimethyl-L-valinamide or a pharmaceutically acceptable salt thereof.

9. A compound according to claim 1 which is N,β,β-trimethyl-L-phenylalanyl-4-benzoyl-N-[(1S,2E)-3-carboxy-1-isopropyl-2-butenyl]-N,β,β-trimethyl-L-phenylalaninamide or a pharmaceutically acceptable salt thereof.

10. A method for identifying a tubulin binding site, which method comprises:
(a) contacting a compound according to claim 1 to a sample comprising tubulin such that the compound is able to irreversibly bind to the tubulin;
(b) separating the tubulin into a plurality of tubulin fragments; and
(c) identifying at least one tubulin fragment with the compound bound thereto,
wherein the identification of a tubulin fragment with the compound bound thereto identifies said fragment as a tubulin binding site.

11. The method according to claim 10 in which the sample is irradiated after contacting the compound to the sample.

12. The method according to claim 10 wherein:
(a) the compound is detectably labeled, and
(b) a tubulin fragment with the compound bound thereto is identified by detecting the label with said fragment.

13. The method according to claim 10 in which the tubulin is chemically digested.

14. The method according to claim 13 in which the tubulin is chemically digested with formic acid or CNBr.

15. The method according to claim 10 in which the tubulin is enzymatically digested.

16. The method according to claim 15 in which the tubulin is digested with Lys C, Trypsin or subtilisin.

17. The method according to claim 10 in which the compound is 4-benzoyl-N,β,β-trimethyl-L-phenylalanyl-N1-[(1S,2E)-3-carboxy-1-isopropylbut-2-enyl]-N1,3-dimethyl-L-valinamide or a pharmaceutically acceptable salt thereof.

18. The method according to claim 10 in which the compound is N,β,β-trimethyl-L-phenylalanyl-4-benzoyl-N-[(1S,2E)-3-carboxy-1-isopropyl-2-butenyl]-N,β,β-trimethyl-L-phenylalaninamide or a pharmaceutically acceptable salt thereof.

19. A method for identifying a hemiasterlin competitor, which method comprises:
(a) contacting a test compound and a probe to a sample comprising tubulin, wherein the probe
(i) is a compound according to claim 1, and
(ii) is contacted to the sample such that it is able to irreversible bind tubulin;
(b) detecting binding of the probe to the tubulin; and
(c) comparing said binding to binding of the probe to tubulin in the absence of the test compound,
in which reduced binding in the presence of the test compound identifies said test compound as a hemiasterlin competitor.

20. The method according to claim 19 in which the sample is irradiated after contacting the probe to the sample.

21. The method according to claim 19 in which the test compound is contacted to the sample before the probe.

22. The method according to claim 19 in which the test compound and the probe are contacted to the sample simultaneously.

23. The method according to claim 19 in which the tubulin is digested after being contacted with the probe.

24. The method according to claim 23 in which the tubulin is chemically digested.

25. The method according to claim 24 in which the tubulin is digested with formic acid or CNBr.

26. The method according to claim 24 in which the tubulin is enzymatically digested.

27. The method according to claim 26 in which the tubulin is digested with Lys C, Trypsin or subtilisin.

28. The method according to claim 10 in which the compound is 4-benzoyl-N,β,β-trimethyl-L-phenylalanyl-N1-[(1S,2E)-3-carboxy-1-isopropylbut-2-enyl]-N1,3-dimethyl-L-valinamide or a pharmaceutically acceptable salt thereof.

29. The method according to claim 10 in which the compound is N,β,β-trimethyl-L-phenylalanyl-4-benzoyl-N-[(1S,2E)-3-carboxy-1-isopropyl-2-butenyl]-N,β,β-trimethyl-L-phenylalaninamide or a pharmaceutically acceptable salt thereof.


SEQUENCE LISTING (36 sequences)

SEQ ID NO: 1 - protein, 35 aa, Rattus norvegicus
RECISIHVGQAGVQIGNACWELYCLEHGIQPDGQM

SEQ ID NO: 2 - protein, 66 aa, Rattus norvegicus
PSDKTIGGGDDSFNTFFSETGAGKHVPRAVFVDLEPTVIDEVRTGTYRQLFHPEQLITGKEDAANN

SEQ ID NO: 3 - protein, 49 aa, Rattus norvegicus
ERLSVDYGKKSKLEFSIYPAPQVSTAVVEPYNSILTTHTTLEHSDCAFM

SEQ ID NO: 4 - protein, 66 aa, Rattus norvegicus
VDNEAIYDICRRNLDIERPTYTNLNRLISQIVSSITASLRFDGALNVDLTEFQTNLVPYPRIHFPL

SEQ ID NO: 5 - protein, 11 aa, Rattus norvegicus
VKCDPGHGKYM

SEQ ID NO: 6 - protein, 64 aa, Rattus norvegicus
ACCLLYRGDVVPKDVNAAIATIKTKRTIQFVDWCPTGFKVGINYQPPTVVPGGDLAKVQRAVCM

SEQ ID NO: 7 - protein, 21 aa, Rattus norvegicus
LSNTTAIAEAWARLDHKFDLM

SEQ ID NO: 8 - protein, 15 aa, Rattus norvegicus
YAKRAFVHWYVGEGM

SEQ ID NO: 9 - protein, 12 aa, Rattus norvegicus
EEGEFSEAREDM

SEQ ID NO: 10 - protein, 26 aa, Rattus norvegicus
AALEKDYEEVGVDSVEGEGEEEGEEY

SEQ ID NO: 11 - protein, 40 aa, Rattus norvegicus
MRECISIHVGQAGVQIGNACWELYCLEHGIQPDGQMPSDK

SEQ ID NO: 12 - protein, 20 aa, Rattus norvegicus
TIGGGDDSFNTFFSETGAGK

SEQ ID NO: 13 - protein, 36 aa, Rattus norvegicus
HVPRAVFVDLEPTVIDEVRTGTYRQLFHPEQLITGK

SEQ ID NO: 14 - protein, 16 aa, Rattus norvegicus
EDAANNYARGHYTIGK

SEQ ID NO: 15 - protein, 12 aa, Rattus norvegicus
EIIDLVLDRIRK

SEQ ID NO: 16 - protein, 39 aa, Rattus norvegicus
LADQCTGLQGFLVFHSFGGGTGSGFTSLLMERLSVDYGK

SEQ ID NO: 17 - protein, 114 aa, Rattus norvegicus
LEFSIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLATYAPVISAEK

SEQ ID NO: 18 - protein, 24 aa, Rattus norvegicus
AYHEQLSVAEITNACFEPANQMVK

SEQ ID NO: 19 - protein, 7 aa, Rattus norvegicus
CDPRHGK

SEQ ID NO: 20 - protein, 15 aa, Rattus norvegicus
YMACCLLYRGDVVPK

SEQ ID NO: 21 - protein, 10 aa, Rattus norvegicus
DVNAAIATIK

SEQ ID NO: 22 - protein, 14 aa, Rattus norvegicus
RSIQFVDWCPTGFK

SEQ ID NO: 23 - protein, 18 aa, Rattus norvegicus
VGINYQPPTVVPGGDLAK

SEQ ID NO: 24 - protein, 24 aa, Rattus norvegicus
VQRAVCMLSNTTAIAEAWARLDHK

SEQ ID NO: 25 - protein, 7 aa, Rattus norvegicus
FDLMYAK

SEQ ID NO: 26 - protein, 29 aa, Rattus norvegicus
RAFVHWYVGEGMEEGEFSEAREDMAALEK

SEQ ID NO: 27 - protein, 21 aa, Rattus norvegicus
DYEEVGVDSVEGEGEEEGEEY

SEQ ID NO: 28 - protein, 449 aa, Homo sapiens
MRECISVHVGQAGVQIGNACWELFCLEHGIQADGTFDAQASKINDDDSFTTFFSETGNGKHVPRAVMIDLEPTVVDEVRAGTYRQLFHPEQLITGKEDAANNYARGHYTVGKESIDLVLDRIRKLTDACSGLQGFLIFHSFGGGTGSGFTSLLMERLSLDYGKKSKLEFAIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLVTYAPIISAEKAYHEQLSVAEITSSCFEPNSQMVKCDPRHGKYMACCMLYRGDVVPKDVNVAIAAIKTKRTIQFVDWCPTGFKVGINYQPPTVVPGGDLAKVQRAVCMLSNTTAIAEAWARLDHKFDLMYAKRAFVHWYVGEGMEEGEFSEAREDLAALEKDYEEVGTDSFEEENEGEEF

SEQ ID NO: 29 - protein, 451 aa, Homo sapiens
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTRLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLATYAPVISAEKAYHEQLSVADITNACFEPANQMVKCDPGHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGVDSVEGEGEEEGEEY

SEQ ID NO: 30 - protein, 447 aa, Macaca fascicularis
RECISVHVGQAGVQMGNACWELYCLEHGIQPDGQMPSDKTIGGGDDSFTTFFCETGAGKHVPRAVFVDLEPTVIDEIRNGPYRQLFHPEQLITGKEDAANNYARGHYTIGKEIIDPVLDRIRKLSDQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIAAIKTKRSIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGIDSYEDEDEGEE

SEQ ID NO: 31 - protein, 442 aa, Macaca mulatta
GQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGVDSVEGEGEEEGEEY

SEQ ID NO: 32 - protein, 449 aa, Mus musculus
MRECISVHVGQAGVQIGNACWELFCLEHGIQADGTFGTQASKINDDDSFTTFFSETGNGKHVPRAVMVDLEPTVVDEVRAGTYRQLFHPEQLITGKEDAANNYARGHYTVGKESIDLVLDRIRKLTDACSGLQGFLIFHSFGGGTGSGFTSLLMERLSLDYGKKSKLEFAIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLVTYAPIISAEKAYHEQLSVAEITSSCFEPNSQMVKCDPRHGKYMACCMLYRGDVVPKDVNVAIAAIKTKRTIQFVDWCPTGFKVGINYQPPTVVPGGDLAKVQRAVCMLSNTTAIAEAWARLDHKFDLMYAKRAFVHWYVGEGMEEGEFSEAREDLAALEKDYEEVGTDSFEEENEGEEF

SEQ ID NO: 33 - protein, 449 aa, Cricetulus griseus
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLATYAPVISAEKAYHEQLTVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGADSAEGDDEGEEY

SEQ ID NO: 34 - protein, 451 aa, Rattus norvegicus
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLMERLSVDYGKKSKLEFSIYPAPQVSTAVVEPYNSILTTHTTLEHSDCAFMVDNEAIYDICRRNLDIERPTYTNLNRLIGQIVSSITASLRFDGALNVDLTEFQTNLVPYPRIHFPLATYAPVISAEKAYHEQLSVAEITNACFEPANQMVKCDPRHGKYMACCLLYRGDVVPKDVNAAIATIKTKRTIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGVDSVEGEGEEEGEEY

SEQ ID NO: 35 - protein, 446 aa, Gallus gallus
MRECISVHIGQAGVQIGNACWELFCLEHSIQPDGTFSDPPSSDDSFATFFRETSMSKYVPRAIMVDLEPTVVDEVRTGTYRHLFHPEQLITGKEDAANNYARGHYTVGKDKVDMVSDRIRKLADSCSGLQGFLIFHSFGGGTGSGFTSLLMERLSVEYGKKSKLEFAIYPAPQASSAVVEPYNSVLTTHTTLEHSDCVFMVDNEAIYDICHRNLDIERPTYTNLNRLISQIVSSITASLRFDGALNVDLTEFQTNLVPFPRIHFPLVTYAPIISSDRAYHEQLSVAEITSSCFEPNNQMVKCDPQQGKYMACCMLYRGDVVPKDVNVAIAAIKTNRSLQFVDWCPTGFKVGINYQPPIPTPGGDLAQVQRAVCMLSNTTAIAEAWARLDHKFDLMYAKRAFVHWYVSEGMEEGEFAEAREDLAALEKDYDEVATDLFEDENEAGDS

SEQ ID NO: 36 - protein, 449 aa, Xenopus laevis
MRECISIHVGQAGVQIGNACWELYCLEHGIQPDGQMPSDKTIGGGDDSFNTFFSETGAGKHVPRAVFVDLEPTVIDEVRTGTYRQLFHPEQLITGKEDAANNYARGHYTIGKEIIDLVLDRIRKLADQCTGLQGFLVFHSFGGGTGSGFTSLLLERLSVDYGKKSKLEFAIYPAPQVSTAVVEPYNSILTTHTTLEHSDCAFMVDNEAIYDICRRNLDIERPTYTNLNRLISQIVSSITASLRFDGALNVDLTEFQTNLVPYPRIHFPLATYAPVISAEKAYHEQLTVADITNACFEPANQMVKCDPRHGKYMACCLLYRGDVVPKDVNAAIATIKTKRSIQFVDWCPTGFKVGINYQPPTVVPGGDLAKVQRAVCMLSNTTAIAEAWARLDHKFDLMYAKRAFVHWYVGEGMEEGEFSEAREDMAALEKDYEEVGADSADAEDEGEEY